(19) United States
(12) Patent Application Publication
(10) Pub. No.: US 2022/0017921 A1
(43) Pub. Date: Jan. 20, 2022

(54) IMPROVED VECTOR SYSTEMS FOR CAS PROTEIN AND SGRNA DELIVERY, AND USES THEREFOR

(71) Applicants: The Broad Institute, Inc., Cambridge, MA (US); Dana-Farber Cancer Institute, Inc., Boston, MA (US)

(72) Inventors: William Nicholas Haining, Boston, MA (US); Juan Dubrot, Cambridge, MA (US); Robert Manguso, Cambridge, MA (US); Kathleen Yates, Boston, MA (US); John Doench, Cambridge, MA (US)

(73) Assignees: The Broad Institute, Inc., Cambridge, MA (US); Dana-Farber Cancer Institute, Inc., Boston, MA (US)

(21) Appl. No.: 17/299,755

(22) PCT Filed: Dec. 4, 2019
(86) PCT No.: PCT/US2019/064555

§ 371 Date: Jun. 3, 2021

Related U.S. Application Data
(60) Provisional application No. 62/775,293, filed on Dec. 4, 2018.
(60) Provisional application No. 62/816,787, filed on Mar. 11, 2019.

(51) Int. Cl. C12N 15/86; C12N 9/22; C12N 15/11

(52) U.S. Cl. CPC C12N 15/86; C12N 9/22; C12N 15/111; C12N 2310/20; C12N 2740/15043; C12N 2830/002; C12N 2840/203; C12N 2740/10043; C12N 2750/14143; C12N 2800/24

(57) ABSTRACT

The present disclosure provides vectors, methods and kits for for delivery and stable expression of CRISPR/Cas components capable of inducing genetic modification of cells, followed by recombinase-mediated excision of some or all of these components after the cells have been successfully genetically modified. The disclosed vectors and methods provide for reduced immunogenic effects arising from one or more CRISPR/Cas components. The disclosed vectors comprise coding sequences that encode a Cas protein, detectable markers and a guide RNA. The disclosed vectors provide for the subsequent genomic excision of the CRISPR/Cas components after successful genetic modification, as mediated by recombinase recognition of recombination sites flanking one or more of the disclosed coding sequences. The present disclosure further provides methods of generating a population of genetically modified tumor cells for screening a candidate target gene for cancer immunotherapy.

### RELATED APPLICATIONS

This application claims priority to U.S. Provisional Patent Application No. 62/775,293, filed Dec. 4, 2018, and U.S. Provisional Patent Application No. 62/816,787, filed Mar. 11, 2019, each of which is incorporated herein by reference.

### FIELD OF THE INVENTION

The present disclosure relates generally to the field of genome editing, and more specifically to improved vectors for delivering CRISPR/Cas and other exogenous transgenes into human and other mammalian cells to genetically modify those cells, and then removing some or all of the transgenes to reduce immunogenic effects of the exogenous transgenes. The improved vector systems have particular application in the generation of large pools of cells with diverse gene knock-outs for functional genomic screening, such as high throughput screens for cancer therapeutics and targets.

### BACKGROUND

Cancer immunotherapy has made noticeable progress in the last decade. After many years of disappointing results, the tide has finally changed and immunotherapy has become a clinically validated treatment for many cancers. Immunotherapeutic strategies include cancer vaccines, oncolytic viruses, adoptive transfer of ex vivo activated T and natural killer cells, and administration of antibodies or recombinant proteins that either co-stimulate cells or block the so-called immune checkpoint pathways. The recent success of several immunotherapeutic regimes, such as monoclonal antibody blocking of cytotoxic T lymphocyte-associated protein 4 (CTLA-4) and programmed cell death protein 1 (PD1), has boosted the development of this treatment modality, with the consequence that new therapeutic targets and schemes which combine various immunological agents are now being described at a breathtaking pace. (Farkona et al. (2016), BMC Medicine 14:73). Several immune checkpoint inhibitors have exhibited promising clinical success. Moreover, there are an increasing number of new potential targets for cancer immunotherapy that are currently being developed both as monotherapy and in combination with others. However, the lack of durable clinical responses, due in part to the resistance mechanisms that tumors exhibit in a significant proportion of patients, urge for novel approaches to find the right therapeutic strategies.

Functional genomics has emerged as a powerful tool that can help to reveal some of these unknown processes. Since its discovery, the CRISPR/Cas system has been widely explored for its utility in cancer research. CRISPR/Cas screens are a powerful functional genomics tool to discover novel targets for cancer therapy. For pooled screening with CRISPR/Cas, a cell population with a diversity of gene knockouts needs to be generated. One main goal of pooled CRISPR/Cas9 screens in cancer research is to identify genotype-specific vulnerabilities. These ‘essential’ genes can be potential drug targets, as their functional depletion leads to reduced viability. These genetically modified cancer cells can also be injected into animals to evaluate cancer behavior in response to certain drugs, such as immune check point inhibitors for cancer immunotherapy.

CRISPR-Cas9 technology has been extensively used in functional genomics to perform genetic screens in various fields. However, the production of such in vivo genetic screens can require the stable expression of components of the CRISPR/Cas9 system, as well as detectable markers, thus requiring genomic integration of these components. Therefore, the Cas/sgRNA components can be introduced or delivered into cancer cells using various stable or integrating vectors, e.g., lentiviral vectors. The resulting cells would express Cas9, the sgRNA, and various detectable markers (e.g., reporter genes, selectable markers, cell surface proteins, and enzymes) that are integrated into their genome by the vector. Unfortunately, in many cases these proteins are immunogenic because they are exogenous to the host, and this fact presents a major obstacle in the context of cancer immunology. The inoculation of such engineered tumor cells into immunocompetent hosts can result in either tumor rejection or an aberrant response to the immunotherapy due to the presence of the foreign proteins, making it difficult to de-convolute the data or even obtain consistent data.

Thus, there exists a need in the art to provide methods of transient and stable delivery of CRISPR-Cas9 components for which these components may be subsequently excised in order to reduce immunogenic effects. A need further exists for methods of screening cancer cells in vivo for target genes that may be candidates in cancer immunotherapy using improved delivery CRISPR-Cas9 delivery vectors that enable subsequent excision of these components.

### SUMMARY OF THE INVENTION

The present disclosure is based, at least in part, upon the recognition that components of CRISPR/Cas systems that are used to produce genetically modified cells (e.g., tumor cells), can cause immunogenicity when the modified cells are inoculated into animals. The enhancement of immunogenicity arising from the overexpression of CRISPR-Cas9 components, often causes tumor rejection and aberrant response to immunotherapy. This phenomenon convolutes the data and renders investigators unable to parse out the true effect of cancer immunotherapy from the immune response elicited by CRISPR-Cas9 components. The invention is also based, at least in part, upon the development of novel strategies in the design of new CRISPR/Cas vector systems that avoid the problem of altered immunogenicity by using a site-specific recombinase system, such as Cre-Lox or Flp-FRT, to excise components of the CRISPR/Cas systems after they have performed their role of genetically modifying the cells. Using this novel strategy, both genome editing capacity of the CRISPR/Cas system and the normal in vivo behavior of the resulting cells can remain largely unaltered.

The disclosed CRISPR/Cas9 components may comprise a Cas protein, a guide RNA (e.g. a single guide RNA or “sgRNA”), and/or selectable or detectable marker proteins. In some embodiments, the disclosed components may comprise a Cas9 protein, an sgRNA, and one or more detectable marker proteins. In some embodiments, the disclosed components may comprise a Cas9 protein, an sgRNA, and two or more detectable marker proteins. The disclosed CRISPR/Cas9 components may consist or consist essentially of a Cas9 protein, an sgRNA, and one or more detectable marker proteins.

The present disclosure provides methods, nucleic acid vectors and kits for stable expression of CRISPR/Cas components for genetic modification of cells. The present disclosure further provides methods, nucleic acid vectors and kits for recombinase-mediated excision of some or all of these exogenous components, as well as accessory components such as selectable or detectable markers, after the cells have been successfully genetically modified that thereby reduce the immunogenic effects of the CRISPR/Cas components.

In principle, any integrating nucleic acid vector capable of delivering CRISPR/Cas components and may be used in accordance with the disclosed methods. In certain spects, the present disclosure provides modified retroviral vectors (e.g., modified lentiviral vectors) that have been adapted for use in recombinant DNA technology, include transgene delivery. The disclosed retroviral vectors may be produced in packaging cell lines. The disclosed retroviral vectors are capable of integration and, thus comprise 5′ and 3′ long terminal repeat (LTR) regions.

Accordingly, in some aspects, provided herein are methods of producing a population of genetically modified cells, comprising i) providing a population of cells, and ii) introducing a first integration vector into a portion of the population of cells. In some embodiments, the first integration vector is a replication defective retroviral vector derived from a primate lentivirus, wherein the first integration vector comprises a first nucleic acid sequence comprising a first promoter operably linked to a Cas protein coding sequence encoding a Cas protein; and a first 3′ site-specific recombination site located 3′ to the Cas coding sequence. The first integrating vector may be capable of integration into the genomes of a portion of the population of cells.

In some embodiments, the disclosed methods further comprise iii) introducing an sgRNA into at least a portion (or all) of the population of cells, wherein the sgRNA is capable of guiding the Cas protein to a target site in the genomes of a portion of the population of cells, and wherein the Cas protein is capable of double-stranded DNA cleavage at the target site; iv) culturing the population of cells for a time sufficient for (a) integration of the first integrating vector into the genomes of a portion of the population of cells; and (b) induction of a genetic modification at the target site in the genomes of a portion of the population of cells by double-stranded DNA cleavage by the Cas protein and the sgRNA; and v) introducing a first recombinase into a portion of the population of cells. In certain embodiments, the first recombinase catalyzes recombination between the first 3′ site-specific recombination site and a first 5′ site-specific recombination site located 5′ to the Cas protein coding sequence, thereby causing excision of the Cas protein coding sequence from the genomes of at least a portion (or all) of the population of cells.

In some embodiments of the disclosed methods, the first 3′ site-specific recombination site is located within a 3′ long terminal repeat (LTR) region at the 3′ end of the first integration vector and is duplicated during integration to produce the first 5′ site-specific recombination site located within a 5′ long terminal repeat (LTR) at the 5′ end of the first integration vector. The first integration vector may further comprise a first 5′ site-specific recombination site located 5′ of at least the Cas protein coding sequence. In some embodiments, the Cas protein is Cas9 or a Cas9 analog.

In some embodiments of the disclosed methods, a single site-specific recombinase may catalyze excision between a pair of site-specific recombination sites in a first integration vector and between a pair of site-specific recombination sites in a second integration vector, such that single site-specific recombinase can be used to induce recombination and excision in both integrated vectors. In some embodiments, the pairs of site-specific recombination sites differ between the two integration vectors (e.g., two pairs of different Lox sites or two pairs of different FRT sites) to reduce the likelihood of recombination, rather than excision, between the integrated vectors.

In some embodiments, the first integrating vector further comprises a second coding sequence encoding a first detectable marker. In certain embodiments, the first coding sequence encoding the Cas protein is operably linked to this second coding sequence, e.g. by a first spacer. The first detectable marker may comprise an antibiotic resistance gene.

In some embodiments, the first spacer comprises a third coding sequence encoding a peptide, which may comprise a cleavage site for one or more proteases. The protease may comprise an endogenous protease, e.g., a P2A peptide or a T2A peptide. Alternatively, the first spacer may comprise an internal ribosome entry site (IRES).

In some embodiments of the disclosed methods, wheein the first 5′ paired site-specific recombination site and the first 3′ paired site-specific recombination site flank the first coding sequence encoding the Cas protein, the second coding sequence encoding the first detectable marker, the first promoter and the enhancer sequence. In some embodiments, the first integrating vector further comprises a second promoter operably linked to a fourth coding sequence encoding a second detectable marker. The first promoter may comprise a constitutive promoter, an inducible promoter or a tissue-specific promoter. In some embodiments, the first integrating vector further comprises a transcription enhancer sequence, e.g., a woodchuck hepatitis virus post-transcriptional regulatory element (WPRE) sequence.

In some embodiments, the sgRNA is delivered into a portion of the population of cells by the first integrating vector. In certain embodiments, the first integrating vector further comprises a U6 promoter operably linked to a fifth coding sequence encoding the sgRNA. The fifth coding sequence encoding the sgRNA may be located at a multiple cloning site of the first integrating vector. In other embodiments, the sgRNA is delivered into a portion of the population of cells by an expression vector.

The genetic modification of the disclosed methods may comprise a disruption of an endogenous gene, wherein the sgRNA is designed to target a nucleic acid sequence of the endogenous gene. In some embodiments, the methods further comprise repairing the double strand break by non-homologous end joining (NHEJ) resulting in the disruption of the endogenous gene. In other embodiments, the genetic modification is an insertion of an exogenous nucleic acid into a target site targeted by the sgRNA. In such embodiments, the methods further comprise introducing to the population of cells a donor sequence, wherein the donor sequence comprises the exogenous nucleic acid flanked by nucleic acid sequences that are homologous to the target site; repairing the double strand break by homologous recombination resulting in the insertion of the exogenous nucleic acid at the target site. The donor sequence may be introduced by calcium phosphate precipitation, liposome transfection, electroporation, or nanoparticles. The donor sequence may be introduced to the population of cells prior to, simultaneously, or after introducing the first integrating vector and the sgRNA.

The first recombinase may be delivered into the population of the cells by a protein, or by a first AAV vector, wherein the first AAV vector comprises a sequence encoding the first recombinase operably linked to a promoter. In other embodiments, the first recombinase is delivered into the population of the cells by a first integrase deficient lentiviral vector, wherein the first integrase deficient lentiviral vector comprises a sequence encoding the first recombinase operably linked to the fourth promoter. The first recombinase may comprise a Cre, and the first site-specific recombination site and the second site specific recombination site may comprise Lox sites. In some embodiments, the Lox site is selected from LoxP, Lox2272, and Lox5171 sites. In other embodiments, the site specific recombination site(s) can be recognized by an FLP, a ΦC31 or a Dre recombinase.

In some embodiments, the first recombinase catalyzes excision of the nucleic acid between the second 5′ paired recombination site and the second 3′ paired recombination site. In certain embodiments, the first site specific recombination site and the second site specific recombination site are different from the second 5′ paired recombination site and the second 3′ paired recombination site. The second recombinase may be delivered into the population of the cells by a second protein, or by a second AAV vector, wherein the second AAV vector comprises a sequence encoding the second recombinase operably linked to a promoter.

In some aspects, provided herein are CRISPR/Cas integrating vectors for use in accordance with the presently disclosed methods. The disclosure provides a first integrating vector comprising a promoter operably linked to a nucleotide sequence encoding a Cas protein; at least two copies of a site-specific recombination site; and at least one nucleotide sequence encoding a selectable marker; and/or an enhancer sequence. The first integrating vctor may comprise a spacer sequence positioned between the nucleotide sequence encoding the Cas and the nucleotide sequence encoding the selectable marker. The disclosure further provides a second integrating vector comprising at least two copies of a site-specific recombination site; a first promoter operably linked to at least one nucleotide sequence encoding an sgRNA; and a second promoter operably linked to at least one nucleotide sequence encoding a selectable marker; and/or an enhancer sequence. The second integrating vector may comprise a lentiviral vector.

The disclosed vectors may further comprise additional elements for recombinations steps following integration of the CRISPR/Cas components. In some embodiments, the disclosed vectors compritse two site-specific recombination sites (e.g., Lox sites) flanking the Cas protein coding sequence that can be recombined by a site-specific recombinase (e.g., Cre) to excise the region between the sites, including the Cas protein coding sequence. By removing the sequences between the site-specific recombination sites, immunogenicity arising from the proteins encoded by the excised sequences may be reduced or eliminated.

Accordingly, the disclosure provides methods and vectors for use in accordance with these methods wherein the first 5′ paired site-specific recombination site and the first 3′ paired site-specific recombination site flank the first coding sequence encoding the Cas protein and the second coding sequence encoding the first detectable marker. In some embodiments, the first 5′ paired site-specific recombination site and the first 3′ paired site-specific recombination site of the disclosed vectors flank the first coding sequence encoding the Cas protein, the second coding sequence encoding the first detectable marker, the first promoter, the fourth coding sequence encoding the second detectable marker, the second promoter, and/or the enhancer sequence.

In some embodiments of the disclosed vectors, at least one of the detectable markers is positioned between the site-specific recombination sites so that excision of the region between the recombination site sequences can be selected or detected. In some embodiments, a single detectable marker is positioned between the site-specific recombination sites and another detectable marker is positioned at a site other than between the recombination site sequences so that integration and excision can be selected or detected separately. In some embodiments, when there are two (or more) detectable markers there will be at least two promoters so that a single promoter is not driving expression of the coding sequences encoding the two (or more) detectable markers and the Cas protein.

The disclosed vectors are especially suitable for high throughput in vivo screening of candidate target genes for cancer immunotherapy. Accordingly, in some aspects, provided herein are methods for generating a population of tumor cells comprising: (i) providing a population of tumor cells; (ii) introducing a first integration vector into at least a portion of the population of tumor cells, wherein the first integration vector comprises a first nucleic acid sequence comprising a first promoter operably linked to a Cas protein coding sequence encoding a Cas protein; and at least a first 3′ site-specific recombination site located 3′ to the Cas coding sequence, and wherein the first integrating vector is capable of integration into the genomes of at least a portion of the population of cells; (iii) introducing a plurality of second integration vectors into at least a portion of the population of tumor cells, wherein each of the plurality of second integration vectors comprises a second nucleic acid sequence encoding an sgRNA, wherein the sgRNA comprises a nucleotide sequence comprising a bar code that corresponds to a candidate target gene, and wherein the sgRNA is capable of guiding the Cas protein to a target site in the genomes of at least a portion of the population of cells, and wherein the Cas protein is capable of double-stranded DNA cleavage at the target site; (iv) culturing the population of tumor cells for a time sufficient for (a) integration of the first integrating vector into the genomes of at least a portion of the population of cells; and (b) induction of a genetic modification at the target site in the genomes of at least a portion of the population of cells by double-stranded DNA cleavage by the Cas protein and the sgRNA; and finally, (v) introducing a first recombinase into at least a portion of the population of cells, wherein the first recombinase catalyzes recombination between the first 3′ site-specific recombination site and a first 5′ site-specific recombination site located 5′ to at least the Cas protein coding sequence, thereby causing excision of the Cas protein coding sequence from the genomes of at least a portion of the population of cells.

Also provided herein are methods of screening the disclosed population of tumor cell to identify a candidate target gene that further comprises grafting a portion of the modified tumor cells of the population onto a mammal; treating the mammal with a monoclonal antibody sufficient to generate an adaptive immune response in the mammal (e.g., a murine mammal, such as a mouse or rat); and isolating the grafted modified tumor cells and sequencing the genomic DNA of the modified tumor cells. In some embodiments of the disclosed methods of screening, each of the first integration vector and each of the plurality of second integration vectors comprises a a replication defective retroviral vector derived from a primate lentivirus. In certain embodiments, the monoclonal antibody is selected from an anti-CTLA4 and an anti-PD-1 monoclonal antibody. In some embodiments, the mammal is immune-competent; in other embodiments, the mammal is immune-deficient or immunocompromised. In some embodiments, the sgRNA of the plurality of second integrating vectors comprises at least 10, at least 25, at least 50, at least 75, at least 100, at least 250, at least 500, at least 750, at least 1,000, or at least 5,000 sgRNAs, wherein each sgRNA comprises a bar code that corresponds to a candidate target gene, and wherein no two bar codes are identical.

In other aspects, provided herein are kits for producing genetically modified cells, comprising: (i) a first integrating vector comprising at least two copies of a first site-specific recombination site; a promoter operably linked to a nucleotide sequence encoding a Cas protein; and at least one nucleotide sequence encoding a selectable marker; (ii) a second integrating vector comprising at least two copies of a second site-specific recombination site; a first promoter operably linked to a nucleotide sequence encoding an sgRNA; a second promoter operably linked to at least one nucleotide sequence encoding a selectable marker. (iii) a third recombinogenic vector comprising a promoter operably linked to a nucleotide sequence encoding a first recombinase, wherein the first recombinase recognizes the first site specific recombination site of the first integrating vector; (ii) a fourth recombinogenic vector comprising a promoter operably linked to a nucleotide sequence encoding a second recombinase, wherein the second recombinase recognizes the second site specific recombination site of the second integrating vector. In some embodiments of the disclosed kits, the first site specific recombination site of the first integrating vector is different from the second site specific recombination site of the second integrating vector. In some embodiments, the third recombinogenic vector comprises an AAV vector or an integrase deficient lentiviral vector. The fourth recominogenic vector may also comprise an AAV vector or an integrase deficient lentiviral vector. In some embodiments, the nucleotide sequence encoding the sgRNA is designed to recognize a target sequence. In some embodiments, the kits comprise a donor nucleotide sequence that comprises a nucleotide sequence to be inserted at the target sequence flanked by two homologous sequences to the target sequence.

Also provided are kits for use in connection with disclosed methods of generating and screening populations of genetically modified tumor cells. In some embodiments, these kits comprise (i) a first integrating vector, comprising at least two copies of a first site-specific recombination site; a promoter operably linked to a nucleotide sequence encoding a Cas protein; and at least one nucleotide sequence encoding a selectable marker; (ii) a plurality of second integrating vectors, each comprising at least two copies of a second site-specific recombination site; a first promoter operably linked to a nucleotide sequence encoding an sgRNA comprising a nucleotide sequence comprising a bar code that corresponds to a candidate target gene; and a second promoter operably linked to at least one nucleotide sequence encoding a selectable marker; a plurality of second integration vectors into at least a portion of the population of tumor cells, (iii) a third vector, comprising a promoter operably linked to a nucleotide sequence encoding a first recombinase, wherein the first recombinase recognizes the first site specific recombination site of the first integrating vector; and (ii) a fourth vector, comprising a promoter operably linked to a nucleotide sequence encoding a second recombinase, wherein the second recombinase recognizes the second site specific recombination site of any of the plurality of second integrating vectors. In certain embodiments of these kits, each of the first integration vector and each of the plurality of second integration vectors comprises a a replication defective retroviral vector derived from a primate lentivirus.

### BRIEF DESCRIPTION OF THE DRAWINGS

FIGS. 1A-1Y are schematic illustrations of various non-limiting examples of vectors to deliver a Cas protein and, optionally, detectable markers into human and other mammalian cells. The vectors include some of all or the following components: a retroviral 5′ long terminal repeat (“5′ LTR”), a retroviral 3′ long terminal repeat (“3′ LTR”), a Cas protein coding sequence (“Cas”), a first promoter (“Promoter 1”), a second promoter (“Promoter 2”), a first detectable marker coding sequence (“Detectable Marker 1”), a second detectable marker coding sequence (“Detectable Marker 2”), at least one site-specific recombination site (“RS”), and one or more spacer (“Spacer”) sequences.

FIGS. 2A-2R are schematic illustrations of various non-limiting examples of vectors to deliver a sgRNA protein into human and other mammalian cells. The vectors include some or all of the following components: an optional retroviral 5′ long terminal repeat (“5′ LTR”), a optional retroviral 3′ long terminal repeat (“3′ LTR”), an sgRNA coding sequence (“sgRNA”), a U6 promoter (“U6”), a third promoter (“Promoter 3”), a third detectable marker coding sequence (“Detectable Marker 3”), a fourth detectable marker coding sequence (“Detectable Marker 4”), at least one site-specific recombination site (“RS”), and one or more spacer (“Spacer”) sequences.

FIGS. 3A-3E are graphs showing stable expression of CRISPR components in cancer cells induces either tumor rejection or exaggerated responses to anti-PD-1 treatment. FIGS. 3A-3C show that transduced CT26 cells (FIG. 3A), D4m3a cells (FIG. 3B) and KPC cells (FIG. 3C), which stably express Cas9 and sgRNA, can induce in vivo tumor rejection and a hyper reaction to anti-PD-1 treatment. Unmodified CT26 cells, D4m3a cells and KPC cells were used as negative control. FIGS. 3D-3E show Cas9 expressing CT26 cells (FIG. 3D) and D4m3a cells (FIG. 3E) induce more tumor rejection and exaggerated response to anti-PD-1 treatment compared to sgRNA expressing CT26 cells and D4m3a cells. Unmodified CT26 cells and D4m3a cells were used as negative control.

FIGS. 4A-4C are exemplary illustrations of vectors delivering Cas9 (FIG. 4A), sgRNA (FIG. 4B), and the recombinase (FIG. 4C). “Drug®” refers to a drug resistant gene driven by promoter 2, e.g., a bls gene that is resistant to blasticidin.

FIGS. 5A-5D are exemplary illustration of various versions of the Cas9 vectors and sgRNA vectors to be used. FIGS. 5A-5B are charts showing successful transduction of CT26 cells to express Cas9 and sgRNA using the exemplary vectors, as evidenced by GFP and mKate expression. FIG. 5C-5D are flow cytometry charts showing successful knock out of CD47 in transduced CT26 cells, which express Cas9 and CD47 sgRNA.

FIG. 6A is a schematic illustration of an integration deficient lentiviral vector carrying Cre recombinase under an EFS promoter. FIG. 6B and FIG. 6C are flow cytometry charts showing the loss of GFP/mKate signal after Cre expression in cells transduced with Cas9_2A_Blast® (FIG. 6B) or Cas9_2A_GFP (FIG. 6C), indicating successful genome excision of Cas9 and the detectable markers.

FIG. 7A depicts various charts which show that Cas9/sgRNA-expressing tumors (FIG. 7A, middle) were rejected or exhibited an abnormal growth compared to unmodified cells (FIG. 7A, left), whereas Cre-infected cells (FIG. 7A, right) showed normal tumor growth in both untreated (dotted lines) and anti-PD-1-treated (solid lines) conditions. FIG. 7B shows Cas9/sgRNA expression did not have any impact in immunodeficient (NSG) mice.

FIG. 8A is a schematic illustration of the pooled genetic screening for identification of target genes in vivo for cancer immunotherapy. FIG. 8B shows tumor volume from NSG mice, wild type untreated mice and wild type anti-PD-1 and anti-CTLA-4 treated mice. FIG. 8C is a volcano plot showing in response to cancer immunotherapy, the enriched genes (left) and depleted genes (right) identified using the method of FIG. 8A.

### DETAILED DESCRIPTION OF THE INVENTION

### Definitions

All scientific and technical terms used herein, unless otherwise defined below, are intended to have the same meaning as commonly understood by one of ordinary skill in the art to which this invention belongs. In the case of any conflict, the present specification, including definitions, will control. References to techniques employed herein are intended to refer to the techniques as commonly understood in the art, including variations on those techniques or substitutions of equivalent or later-developed techniques which would be apparent to one of skill in the art. In order to more clearly and concisely describe the subject matter which is the invention, the following definitions are provided for certain terms which are used in the specification and appended claims.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the recitation of a numerical range for a variable is intended to convey that the invention can be practiced with the variable equal to any of the values within that range. Thus, for a variable that is inherently discrete, the variable can be equal to any integer value within the numerical range, including the end-points of the range. Similarly, for a variable that is inherently continuous, the variable can be equal to any real value within the numerical range, including the end-points of the range. As an example, and without limitation, a variable that is described as having values between 0 and 2 can take the values 0, 1 or 2 if the variable is inherently discrete, and can take the values 0.0, 0.1, 0.01, 0.001, or any other real values 0 and 2 if the variable is inherently continuous.

As used herein, the term “bar code” refers to a short nucleotide sequence identifier comprised within an guide RNA sequence, wherein the gRNA also comprises a sequence that has complementarity to a target gene. A cell that has been transduced with a guide RNA that contains a bar code sequence may be detected by probing a population of cells for the presence of the sequence, thereby conveying the location of the target gene.

As used herein, the terms “genetic modification” and “gene editing” are used interchangeably and refer to the modification of a genetic sequence in a chromosome. Gene editing methods typically involve the use of an endonuclease that is capable of cleaving a target region in a chromosome (e.g., an exon of coding sequence). After cleavage, repair of double-strand breaks by non-homologous end joining in the absence of a template nucleic acid can result in mutations (e.g., insertions, deletions and/or frameshifts) at the target site. Alternatively, in the presence of a donor sequence homologous to sequences flanking the cleavage site, homologous recombination can repair the double-strand breaks with the introduction of an insertion of sequences from the donor sequence (e.g., missense mutations or transgenes). Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), homing endonucleases (e.g., ARC homing endonucleases), meganucleases (e.g., mega-TALs), or a combination thereof. Various gene editing systems using meganucleases, including modified meganucleases, have been described in the art; see, e.g., the reviews by Steentoft et al. (2014), Glycobiology 24(8):663-80; Belfort and Bonocora (2014), Methods Mol Biol. 1123:1-26; Hafez and Hausner (2012), Genome 55(8):553-69; and references cited therein.

As used herein, the term “CRISPR” or “CRISPR/Cas system” refers to an endonuclease comprising a Cas protein, such as Cas9, and a guide RNA that directs DNA cleavage by the Cas protein at a recognition site in the genomic DNA recognized by the guide RNA. Thus, the Cas component of a CRISPR/Cas system is an RNA-guided DNA endonuclease. CRISPR biology, as well as Cas endonuclease sequences and structures, are well known to those of skill in the art (see, e.g., “Complete genome sequence of an M1 strain of Streptococcus pyogenes.” Ferretti J. J., et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607 (2011); and “A programmable dual-RNA-guided DNA endonuclease in adaptive bacterial immunity.” Jinek M., et al., Science 337:816-821 (2012), the entire contents of each of which are incorporated herein by reference). Cas orthologs (e.g., cas9 orthologs) have been described in various species, including, but not limited to, S. pyogenes, S. thermophiles, C. ulcerans, S. diphtheria, S. syrphidicola, P. intermedia, S. taiwanense, S. iniae, B. baltica, P. torquis, S. thermophiles, L. innocua, C. jejuni, G. thermodenitrificans and N. meningitidis. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737, the entire contents of which are incorporated herein by reference.

As used herein, the terms “guide RNA,” “single guide RNA” or “sgRNA” refer to an artificial RNA sequence that can be used to guide a Cas protein (e.g., Cas9) to a target sequence on a chromosome which shares homology with a portion of the sgRNA. sgRNAs are artificial constructs which combine the structures and functions of the naturally-occurring CRISPR RNA (crRNA) and transactivating CRISPR RNA (tracrRNA) found in natural CRISPR systems (e.g., Streptococcus pyogenes CRISPR/Cas9) and which can be sequence-modified to target any desired target sequence.

As used herein, the term “delivery vector” means a system for introducing a desired exogenous nucleic acid into a cell or tissue. Such vectors include viral vectors (e.g., SV40, AAV, lentiviral vectors), liposomes, polymers, biolistic particles (e.g., gold), nanoparticles, and chemical agents (e.g., calcium phosphate).

As used herein, the term “viral vector” refers to a vector derived from a virus that is incapable of replication but is capable of integration into a host cell chromosome, thereby delivering genetic material into the genome of cells inside a living organism (in vivo) or in cell culture (in vitro). Delivery of genes and/or other genetic sequences by a viral vector is termed transduction and the infected cells are described as transduced. Viral vectors can include, without limitation, retroviral vectors (including lentiviral vectors), adenoviral vectors, adeno-associated viral vectors (AAV) and hybrids. The terms “lentiviral vector” and “lentivector” can be used interchangeably to describe viral vectors derived from lentivirus. Viral vectors can be packaged in a viral capsid (by viral proteins expressed from packaging plasmids or by a packaging cell line) or can comprise naked nucleic acid molecules.

As used herein, the term “expression vector” means a single-stranded or double-stranded, linear or circular, nucleic acid that comprises nucleotide sequences that are capable of transcription and translation of a polypeptide-encoding sequence in a given host cell. Expression vectors can integrate into a host cell chromosome or can exist independently of host chromosomes as episomes. Non-integrative expression vectors can include regulatory elements such as operators, enhancers, promoters, transcription initiation, transcriptional termination, translation initiation, ribosomal binding site, and polyadenylation sequences that are necessary or useful for the transcription and translation of the polypeptide-coding sequences. Integrative expression vectors, can also include all or some of these elements as well as integrase coding sequences, long terminal repeats (LTRs) and other sequences necessary or useful for integration. Expression vectors can be derived from bacterial plasmids, viral genomes, or combinations of elements from various bacterial, viral or eukaryotic genomes.

As used herein, “recombinogenic vector” means a retroviral vector which (in its integrated or proviral form) includes at least two site-specific recombination sites which are capable of enzyme-mediated recombination to excise the sequence(s) between them.

As used herein, the terms “polynucleotide”, “nucleotide”, “nucleotide sequence”, “nucleic acid” and “oligonucleotide” can be used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, introns, exons, single guide RNA (sgRNA), messenger RNA (mRNA), cDNA, recombinant polynucleotides, plasmids, vectors, isolated DNA of any sequence, isolated RNA of any sequence, nucleic acid probes, and primers. A polynucleotide can comprise one or more modified nucleotides, such as methylated nucleotides and nucleoside analogs. If present, modifications to the nucleotide structure can be imparted before or after assembly of the polymer.

As used herein, the terms “sequence that encodes” and “coding sequence” are used interchangeably and refers to a deoxyribonucleotide sequence that specifies the ribonucleotide sequence of a functional RNA (e.g., mRNA, tRNA, rRNA, guide RNA) and/or that, through the genetic code, specifies the amino acid sequence of a protein. A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus and a translation stop/nonsense codon at the 3′ terminus.

As used herein, the terms “DNA regulatory region,” “control elements,” and “regulatory elements,” are used interchangeably and refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence (e.g., Cas coding sequence) and/or regulate translation of an encoded polypeptide.

As used herein, a “promoter” or “promoter sequence” is a DNA regulatory region capable of binding an RNA polymerase and initiating transcription of a downstream (3′ direction) coding or non-coding sequence. For purposes of defining the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including constitutive and inducible promoters, can be used in the present disclosure. Exemplary promoters of the disclosure include the EF1α and U6 promoters.

As used herein, the terms “multiple cloning site” and “polylinker” are used interchangeably and refer to a cluster of restriction endonuclease recognition sites on a nucleic acid construct (e.g., a viral vector, transfer vector, expression vector, or naked RNA or DNA).

As used herein, a “polycistronic” genetic locus or mRNA refers to a genetic locus or mRNA that comprises two or more coding sequences (i.e., cistrons) and encodes two or more corresponding proteins.

As used herein, the term “spacer” refers to a polynucleotide sequence between two or more coding sequences in a polycistronic genetic locus or polycistronic mRNA that causes the two or more coding sequences to be translated into two or more corresponding proteins as opposed to a single protein. Examples of spacers include internal ribosome entry site (IRES) elements as well as self-cleaving peptide elements (e.g., T2A, P2A, E2A or F2A elements).

A cell has been “transformed” or “transfected” or “transduced” by exogenous DNA, e.g., a lentiviral vector, when such DNA has been introduced inside the cell. The presence of the exogenous DNA can result in either a permanent or transient genetic change. The transforming DNA either can be integrated (covalently inserted) into the genome of the cell or can exist independently (e.g., as an episome). With respect to eukaryotic cells, a stably transformed cell is one in which the transforming DNA has become integrated into a chromosome so that it is inherited by daughter cells through chromosome replication.

As used herein, the term “host cell” refers to a human or other mammalian cell, including but not limited to non-human primate, rodent (e.g., mouse, rat, hamster), leporidae (e.g., rabbit hare), ovine, bovine, caprine, equine, canine, and feline cells, that is transformed, transfected or transduced with one or more of the vectors of the invention.

As used herein, the term “tumor cell” refers to any well-known cancer cell line. Exemplary tumor cells include the CT26, D4m3a and KPC cell line.

As used herein, the term “target DNA” refers to a DNA polynucleotide that comprises a “target site” or “target sequence.” The terms “target site” or “target sequence” are used interchangeably herein to refer to a nucleic acid sequence present in a target DNA to which a DNA-targeting segment of a guide RNA (e.g., an sgRNA) will bind, provided suitable conditions for binding exist. For example, the target site (or target sequence) 5′-GAGCATATC-3′ (SEQ ID NO: 1) within a target DNA can be targeted by (or be bound by, or hybridize with) the RNA sequence 5′-GAUAUGCUC-3′ (SEQ ID NO: 2). Suitable DNA/RNA binding conditions include physiological conditions normally present in a host cell or its nucleus. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand.”

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends.

As used herein, the terms “nuclease” and “endonuclease” are used interchangeably herein to mean an enzyme which possesses endonucleolytic catalytic activity for DNA cleavage.

As used herein, the terms “sequence-specific recombinase” and “site-specific recombinase” refer to enzymes that specifically recognize and bind to a nucleic acid sites or nucleic acid sequences and catalyze recombination of the nucleic acid(s) at these sites.

As used herein, the terms “sequence-specific recombinase target site”, “site-specific recombinase target site” and “site-specific recombination sites” are used interchangeably and refer to nucleic acid sites or sequences which are recognized by a sequence- or site-specific recombinase and which become the crossover regions during the site-specific recombination event. Examples of sequence-specific recombinase target sites include, but are not limited to, lox sites, frt sites, attL/attR sites, rox sites and dif sites.

As used herein, the term “lox site” refers to a nucleotide sequence at which the product of the cre gene of bacteriophage Pl, Cre recombinase, can catalyze a site-specific recombination. A variety of lox sites are known to the art including but not limited to the naturally occurring loxP (the sequence found in the P1 genome), loxB, loxL and loxR (these are found in the E. coli chromosome) as well as a number of mutant or variant lox sites such as loxP511, lox2272, loxΔ86, loxΔ117, loxC2, loxP2, loxP3 and loxP23. The term “frt site” as used herein refers to a nucleotide sequence at which the product of the FLP gene of the yeast 2 μm plasmid, FLP recombinase, can catalyze a site-specific recombination.

Vector Designs for CRISPR/Cas Integrating Vectors

The present disclosure provides integrating vectors capable of delivering the desired transgenes. In some embodiments, these vectors comprise modified retroviral vectors (e.g., modified lentiviral vectors) that have been adapted for use in recombinant DNA technology, include transgene delivery. Notably, the retroviral vectors are typically replication defective because they lack functional copies of one or more of the loci necessary for capsid production, genome replication and/or genome packaging within the capsid. These vectors may be produced in packaging cell lines which supply the missing functions. However, for use in the present disclosure, the retroviral vectors may be capable of integration and, therefore, may include 5′ and 3′ long terminal repeat (LTR) regions. Integrase and reverse transcriptase are encoded by the pol gene. The gene products are supplied during viral production through a packaging plasmid (i.e. psPAX2, Addgene)

Commonly-used retroviral vectors typically include a variety of other modifications which are necessary or useful for cloning, replication, expression, selection or detection. For example, multiple origins of replication can be included for cloning in different systems, multiple cloning sites (MCS) can be included for inserting transgenes or regulatory elements, enhancer sequences can be included to drive higher levels of expression of desired transgenes, spacers can be included to separate coding sequences under the control of the same promoter, and selectable or detectable marker genes can be included to select for or monitor successfully transformed cells.

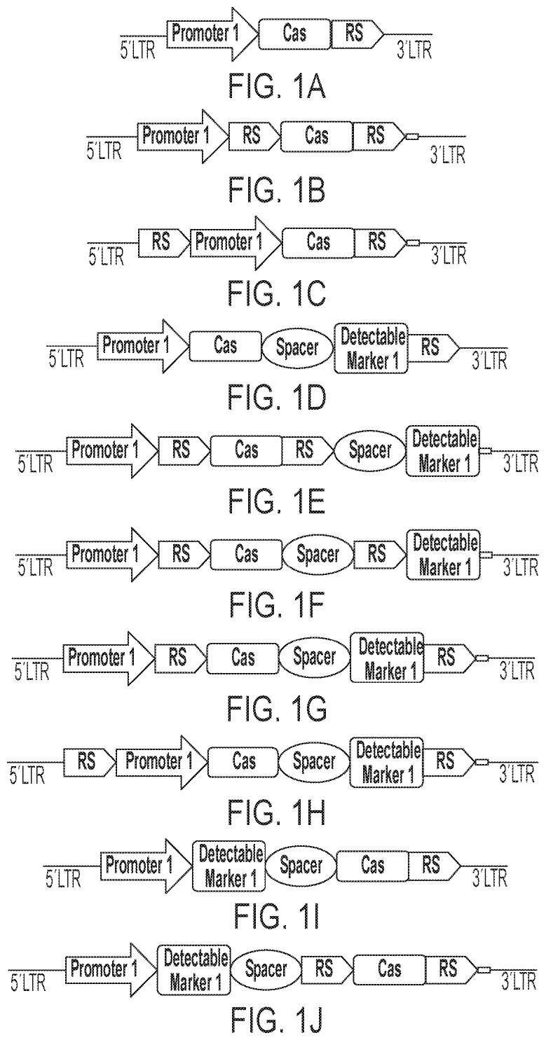

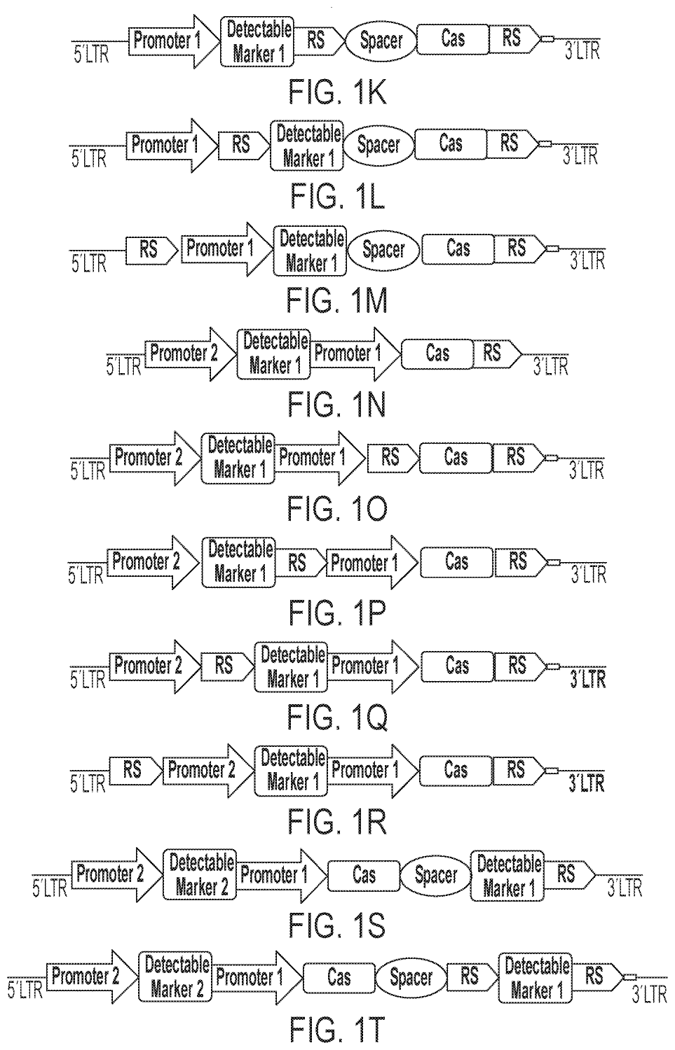

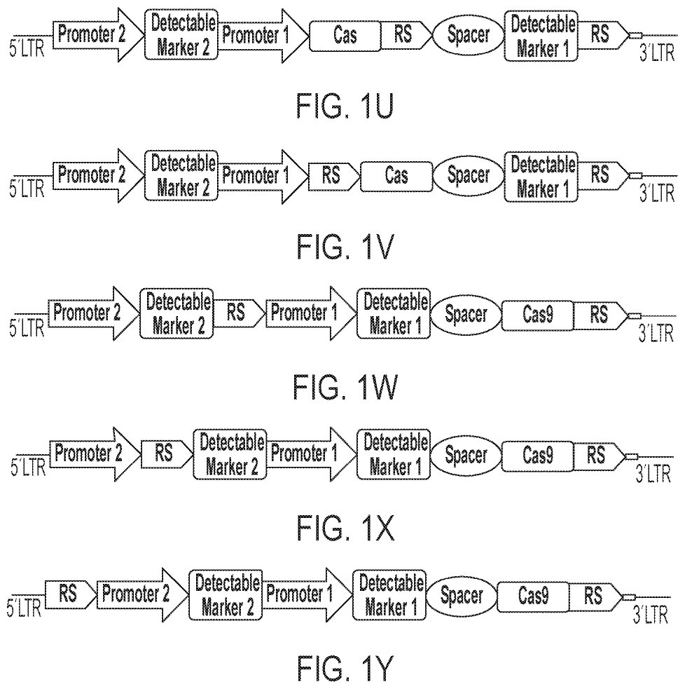

As shown in FIG. 1A, an exemplary integrating CRISPR/Cas vector includes at least the following: a 5′ long terminal repeat (“LTR”) region at the 5′ end of the vector, a first promoter (“Promoter 1”) operably linked to a Cas protein coding sequence (“Cas”) that encodes the chosen Cas protein, at least a first 3′ site-specific recombination site (“RS”) located 3′ to the Cas coding sequence, and a 3′ LTR region at the 3′ end of the vector. Although 5′ LTR may be required for the vector, it does not integrate in the host cell. 3′ LTR is duplicated before integration but it has a deletion on the U3 region (self-inactivating or SIN vector) in the more commonly used lentiviral vectors increasing its safety.

In this embodiment, an exogenous promoter may be required for transgene expression. It may induce expression of the transfer vector if 3′ LTR sequence is intact. If the first 3′ site-specific recombination site is located within the 3′ LTR region, it will be duplicated when the vector integrates into the host cell genome, thereby producing a first 5′ site-specific recombination site. Therefore, a minimal vector, as shown in FIG. 1A, need not include a first 5′ site-specific recombination site prior to integration. However, if the first 3′ site-specific recombination site is not within the duplicated 3′ LTR region, a first 5′ RS may be included in the vector between Promoter 1 and Cas, as shown in FIG. 1B, or between the 5′ LTR region and Promoter 1, as shown in FIG. 1C. Thus, for each of the retroviral vectors of FIGS. 1A-1C, there will be two RS sequences flanking at least the Cas coding sequence after integration (and, in the case of FIG. 1C, also flanking Promoter 1). Therefore, when a site-specific recombinase causes recombination between the two RS sequences, at least the Cas coding sequence will be excised from the integrated vector (and, in the case of FIG. 1C, Promoter 1 will also be excised).

As noted above, the vectors of the invention can optionally include selectable or detectable markers (collectively referred to as “detectable markers” herein) to aid in selecting or detecting cells in which (a) the vector has integrated and/or (b) the region between the site-specific recombination sites has been excised.

FIGS. 1D-1H show embodiments in which the first detectable marker (“Detectable Marker 1”) is located 3′ of the Cas coding sequence and is separated from the Cas sequence by at least a spacer element (“Spacer”).

FIG. 1D shows a construct (as in FIG. 1A) in which there is a single RS sequence within the 3′ LTR region which will be duplicated by reverse transcription (as in FIG. 1A). From 5′ to 3′, the retroviral vector of FIG. 1D comprises the 5′ LTR, followed by Promoter 1, followed by Cas, followed by the Spacer, followed by Detectable Marker 1, followed by the first 3′ RS sequence within the 3′ LTR region.

FIGS. 1E-1H show alternative constructs in which there are two RS sequences because the 3′ RS is not within the duplicated region of the 3′ LTR region.

Thus, from 5′ to 3′, the retroviral vector of FIG. 1E comprises the 5′ LTR, followed by Promoter 1, followed by the 5′ RS sequence, followed by Cas, followed by the 3′ RS sequence, followed by the Spacer, followed by Detectable Marker 1, followed by the 3′ LTR region.

From 5′ to 3′, the retroviral vector of FIG. 1F comprises the 5′ LTR, followed by Promoter 1, followed by the 5′ RS sequence, followed by Cas, followed by the Spacer, followed by the 3′ RS sequence, followed by Detectable Marker 1, followed by the 3′ LTR region.

From 5′ to 3′, the retroviral vector of FIG. 1G comprises the 5′ LTR, followed by Promoter 1, followed by the 5′ RS sequence, followed by Cas, followed by the Spacer, followed by Detectable Marker 1, followed by the 3′ RS sequence, followed by the 3′ LTR region.

From 5′ to 3′, the retroviral vector of FIG. 1H comprises the 5′ LTR, followed by the 5′ RS sequence, followed by Promoter 1, followed by Cas, followed by the Spacer, followed by Detectable Marker 1, followed by the 3′ RS sequence, followed by the 3′ LTR region.

FIGS. 1I-M show embodiments in which the first detectable marker (“Detectable Marker 1”) is located 5′ of the Cas coding sequence and is separated from the Cas sequence by at least a spacer element (“Spacer”).

Thus, FIG. 1I shows a construct (as in FIG. 1A) in which there is a single RS sequence within the 3′ LTR region which will be duplicated by reverse transcription (as in FIG. 1A). From 5′ to 3′, the retroviral vector of FIG. 1I comprises the 5′ LTR, followed by Promoter 1, followed by Detectable Marker 1, followed by the Spacer, followed by Cas, followed by the first 3′ RS sequence within the 3′ LTR region.

Alternatively, FIGS. 1J-1M show constructs in which there are two RS sequences because the 3′ RS is not within the duplicated region of the 3′ LTR region.

Thus, from 5′ to 3′, the retroviral vector of FIG. 1J comprises the 5′ LTR, followed by Promoter 1, followed by Detectable Marker 1, followed by the Spacer, followed by the 5′ RS sequence, followed by Cas, followed by the 3′ RS sequence, followed by the 3′ LTR region.

From 5′ to 3′, the retroviral vector of FIG. 1K comprises the 5′ LTR, followed by Promoter 1, followed by Detectable Marker 1, followed by the 5′ RS sequence, followed by the Spacer, followed by Cas, followed by the 3′ RS sequence, followed by the 3′ LTR region.

From 5′ to 3′, the retroviral vector of FIG. 1L comprises the 5′ LTR, followed by Promoter 1, followed by the 5′ RS sequence, followed by Detectable Marker 1, followed by the Spacer, followed by Cas, followed by the 3′ RS sequence, followed by the 3′ LTR region.

From 5′ to 3′, the retroviral vector of FIG. 1M comprises the 5′ LTR, followed by the 5′ RS sequence, followed by Promoter 1, followed by Detectable Marker 1, followed by the Spacer, followed by Cas, followed by the 3′ RS sequence, followed by the 3′ LTR region.

In other embodiments, some of which are shown in FIGS. 1N-1R, vectors of the invention can include an additional sequence encoding a second promoter (“Promoter 2”) that drives expression of Detectable Marker 1 and which is separate from the Promoter 1 for the Cas coding sequence. As in the embodiments described above, the 5′ SR can be omitted (because the 3′ SR is located within the 3′ LTR region) (FIG. 1N) or can be located in various positions 5′ of the Cas sequence (FIGS. 1O-1R) such that excision of the region between the site-specific recombination sites removes more or fewer components of the integrated vector.

Thus, from 5′ to 3′, the retroviral vector of FIG. 1N comprises the 5′ LTR, followed by Promoter 2, followed by Detectable Marker 1, followed by Promoter 1, followed by Cas, followed by the 3′ RS sequence, followed by the 3′ LTR region.

From 5′ to 3′, the retroviral vector of FIG. 1O comprises the 5′ LTR, followed by Promoter 2, followed by Detectable Marker 1, followed by Promoter 1, followed by the 5′ RS sequence, followed by Cas, followed by the 3′ RS sequence, followed by the 3′ LTR region.

From 5′ to 3′, the retroviral vector of FIG. 1P comprises the 5′ LTR, followed by Promoter 2, followed by Detectable Marker 1, followed by the 5′ RS sequence, followed by Promoter 1, followed by Cas, followed by the 3′ RS sequence, followed by the 3′ LTR region.

From 5′ to 3′, the retroviral vector of FIG. 1Q comprises the 5′ LTR, followed by Promoter 2, followed by the 5′ RS sequence, followed by Detectable Marker 1, followed by Promoter 1, followed by Cas, followed by the 3′ RS sequence, followed by the 3′ LTR region.

From 5′ to 3′, the retroviral vector of FIG. 1R comprises the 5′ LTR, followed by the 5′ RS sequence, followed by Promoter 2, followed by Detectable Marker 1, followed by Promoter 1, followed by Cas, followed by the 3′ RS sequence, followed by the 3′ LTR region.

In variations of the retroviral vectors of FIGS. 1N-1R (not shown), Promoter 2 and Detectable Marker 1 can be located 3′ of the Cas coding sequence. As before, the 5′ RS and 3′ RS can be located at various positions such that excision of the region between the site-specific recombination sites removes more or fewer components of the integrated vector.

In other embodiments, some of which are shown in FIGS. 1S-1Y, vectors of the invention can include an additional sequence encoding a second detectable marker (“Detectable Marker 2”). Detectable Marker 2 can be under the control of Promoter 1, Promoter 2 or a third promoter (“Promoter 3”). Detectable Marker 1 and Detectable Marker 2 can be under the control of the same or different promoters, and one or the other can be under the control of the same promoter as the Cas sequence. Either, both or neither of Detectable Marker 1 and Detectable Marker 2 can be 5′ (or 3′) of the Cas sequence. If any of Detectable Marker 1, Detectable Marker 2 and the Cas sequence are under the control of the same promoter, spacer sequences can be included between them so that the encoded sequences are expressed as separate proteins. In addition, as in the various other embodiments described above, the 5′ RS can be omitted (because the 3′ RS is located within the 3′ LTR region) or the 5′ RS and 3′ RS can be located in various positions such that excision of the region between the site-specific recombination sites removes more or fewer components of the integrated vector.

As will be apparent to one of skill in the art, FIGS. 1A-1Y do not represent all possible variations of the vectors of the invention. In addition to different ordering of the components shown in the figures, additional components such as origins of replication, multiple cloning sites (MCS) or polylinker sites, enhancer sequences, sequences encoding “tags” for proteins, “barcode” sequences, Psi elements etc. can be included. In addition, the vectors will inevitably include sequences derived from the original native vector (e.g., native viral sequences) that are necessary to the function of the vector (e.g., for integration) or that are unnecessary (e.g., inactivated genes for capsid proteins or packaging functions), as well as sequences which are “artifacts” of the process by which the vector was assembled or cloned. For example, for replication defective retroviral vectors that are packaged in capsids, a Psi element may be present near the 5′ LTR but is not shown in the figures for simplicity.

Vectors for Guide RNAs

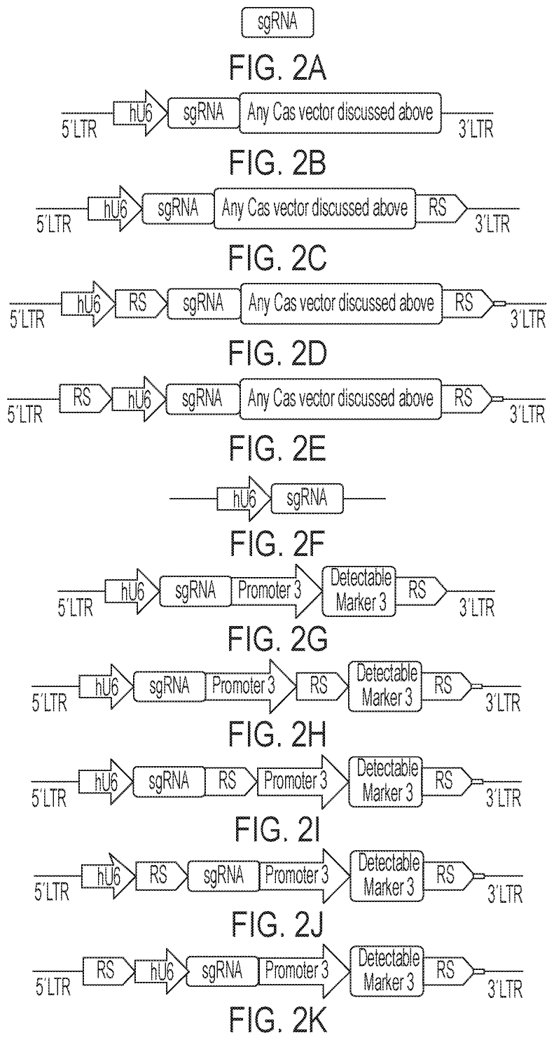

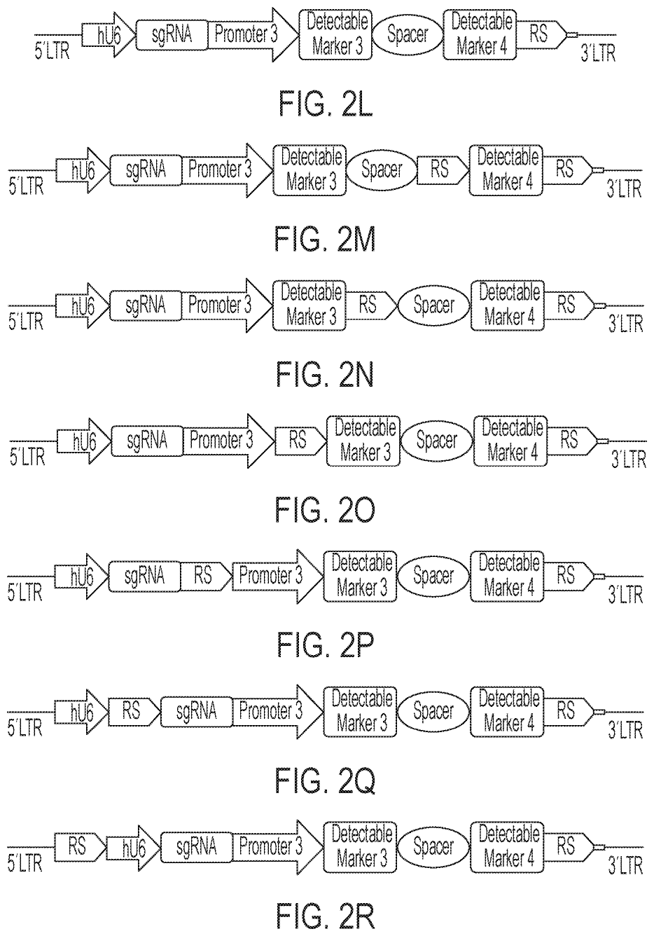

The guide RNAs of the invention can be delivered to host cells in a variety of ways. In the simplest methods, naked RNA molecules (FIG. 2A) can be introduced to cells by methods known in the art, including but not limited to viral vectors (e.g., SV40, AAV, lentiviral vectors), liposomes, polymers, biolistic particles (e.g., gold), nanoparticles, ribonucleoproteins, and chemical agents (e.g., calcium phosphate).

Because the guide RNAs comprise relatively short polynucleotide sequences, it may be possible to encode and express the guide RNAs from the same retroviral vectors as the Cas protein. For example, FIGS. 2B-2E show an sgRNA coding sequence under the control of the human U6 (hU6) promoter at the 5′ end of any of the previously described Cas retroviral vector constructs. Naturally, promoters other than hU6 can be employed, and the sgRNA coding sequence can be 3′ as well as 5′ of the Cas coding sequence, and under the control of the same or different promoters.

However, in some embodiments, it may be desirable to express the guide RNAs from a separate vector. For example, when creating large pools of cells with diverse gene knock-outs for functional genomic screening, it may be convenient to have a single Cas vector which can be co-transfected with a variety of different guide RNA vectors or a large pool of different guide RNA vectors (e.g., with a multiplicity of infection by different guide RNA vectors of at least 10, at least 100, at least 1,000 or at least 10,000 for functional genomic screening).

In some embodiments, the guide RNA vector can be a simple non-integrative expression vector (FIG. 2F) with expression under the control of a constitutive or inducible promoter.

In other embodiments, however, to obtain stable expression of the guide RNA, it may be preferable to use an integrating vector, such as a retroviral vector, including a replication defective retroviral vector. Alternatively, it may be desirable to use an integration defective vector (e.g., an integration deficient lentiviral vector (IDLV)) so that expression of the guide RNA will be limited by the lifetime of the sgRNA vector in vivo.

In addition, as with the Cas vectors discussed above, it may be advantageous to include one or more selectable or detectable markers (collectively referred to as “detectable markers” herein) to identify or select cells in which both the Cas and guide RNA vectors are present.

In some embodiments, the guide RNA vector is a recombinogenic integrating retroviral vector including at least one or two site-specific recombination sites (RS). As described above with respect to the Cas vector, if a 3′ RS site is located within the region of the 3′ LTR that is duplicated during reverse transcription, then the integrated virus will include a 5′ copy of the 3′ LTR region, including a duplication of the 3′ RS to produce a 5′ RS. Alternatively, if the 3′ RS is not within the duplicated 3′ LTR region, a separate 5′ RS may be included. Again, the 5′ RS and 3′ RS can be located in various positions such that excision of the region between the site-specific recombination sites removes more or fewer components of the integrated vector. In the case of guide RNA vectors, in some embodiments the guide RNAs will be less immunogenic than the exogenous detectable marker proteins. Therefore, in some embodiments, the RS sequences can be located such that they flank and mediate the excision of one or more detectable marker coding sequences, but do not flank or mediate excision of the guide RNA coding sequence. However, in other embodiments, the RS sequences can be located such that they flank and mediate the excision of the guide RNA sequences (with or without the detectable markers).

In some embodiments, the guide RNA vector comprises one or more bar code sequences. These bar code sequences may be positioned outside of the at least one or two site-specific RSs, i.e., 5′ of the 5′ RS and 3′ of the 3′ RS.

Non-limiting examples of guide RNA vectors are shown in FIGS. 2A-2R.

As will be apparent to one of skill in the art, FIGS. 2A-2R do not represent all possible variations of the guide RNA vectors of the invention. In addition to different ordering of the components shown in the figures, additional components such as origins of replication, multiple cloning sites (MCS) or polylinker sites, enhancer sequences, sequences encoding “tags” for proteins, “bar code” sequences, Psi elements etc. can be included. In addition, the vectors will inevitably include sequences derived from the original native vector (e.g., native viral sequences) that are necessary to the function of the vector (e.g., for integration) or that are unnecessary (e.g., inactivated genes for capsid proteins or packaging functions), as well as sequences which are “artifacts” of the process by which the vector was assembled or cloned. For example, for replication defective retroviral vectors that are packaged in capsids, a Psi element may be present near the 5′ LTR but is not shown in the figures for simplicity. In the figures the component “hU6” can be a human U6 promoter or any other promoter capable of driving expression of the guide RNA in the host cell. In some embodiments, a constitutive promoter is preferred.

In some embodiments, the RS sequences of the guide RNA vector differ from the RS sequences of the Cas vector. Thus, in some embodiments, the same recombinase (e.g., Cre) can recognize and mediate recombination of the RS sequences of both vectors, but the RS sequences may be different on the two vectors (e.g., loxP511 and lox2272 sites) so that the recombinase does not mediate recombination between the integrated Cas and guide RNA vectors. Alternatively, different recombinases (e.g., Cre and Flp) can recognize and mediate recombination of the RS sequences on the two vectors (e.g., lox and FRT sites). This strategy allows for independent excision of components of one vector (e.g., a guide RNA vector) while leaving the components of the other vector (e.g., a Cas vector) integrated. In some embodiments, this strategy could be used to integrate and excise guide RNA coding sequences sequentially while using the same integrated Cas vector to mediate RNA-guided cleavage and modification of different genetic target sites. After successful completion of all desired genetic modifications, components of the integrated Cas vector could be excised using the appropriate recombinase.

Vectors for Site-Specific Recombinases

Unlike the Cas vectors and the guide RNA vectors of the invention, which may be expressed simultaneously (or at least for over-lapping periods) in the host cells so that the Cas proteins and guide RNAs can act cooperatively to mediate genetic modifications, the recombinase vectors can be expressed after the Cas and guide RNA vectors have performed their roles. In embodiments with different recombinases for the Cas vector and guide RNA vector(s), the different recombinases can be expressed simultaneously or sequentially. In addition, whereas the Cas and guide RNA vectors can be expressed for periods of several days or more, the recombinase vectors can be expressed more transiently.

The site-specific recombinases of the invention can be introduced to the host cells by any means known in the art, including the various delivery vectors described herein. However, because they can be expressed more transiently, in some embodiments non-integrating vectors (e.g., IDLV vectors, smaller expression vectors such as SV40 or AAV vectors) or physical or chemical techniques of introducing nucleic acids (e.g., electroporation, biolistic particles) can be preferred. In addition, although detectable markers can be included in recombinase vectors, such markers may not be necessary if recombinase-mediated excision of Cas vector or guide RNA vector components includes excision of a detectable marker in one of those vectors.

Methods for Genetically Modifying Cells and Pools of Genetically-Modified Cells

The present disclosure also provides methods for producing genetically modified cells using a CRISPR/Cas system with one or more recombinogenic vectors that integrate into host cells, genetically modify the host cells, and then undergo site-specific recombination to excise at least some immunogenic components of the vectors from the genomes of the genetically-modified cells.

In some embodiments, the methods comprise providing a population of cells, introducing any of the recombinogenic Cas vectors (or “first integration vectors”) described above into the cells, introducing at least one guide RNA into the cells, culturing the population of cells for a time sufficient for (a) integration of the first integration vector into the genomes of at least a portion of the population of cells; and (b) induction of a genetic modification at the target site in the genomes of at least a portion of the population of cells by double-stranded DNA cleavage by the Cas protein and the sgRNA; and introducing a first recombinase into at least a portion of the population of cells, wherein the first recombinase catalyzes recombination between the first 3′ site-specific recombination site and a first 5′ site-specific recombination site located 5′ to at least the Cas protein coding sequence, thereby causing excision of the Cas protein coding sequence from the genomes of at least a portion of the population of cells.

In some embodiments of these methods, the guide RNA sequences is introduced by any of the methods described above.

In some embodiments, the guide RNA sequences are introduced by recombinogenic retroviral vectors (“RNA guide vectors” or “second integration vectors”) as described herein. If the same site-specific recombinase can catalyze excision between the pair of site-specific recombination sites in the first integration vector and between the pair of site-specific recombination sites in the second integration vector, then that single site-specific recombinase can be used to induce recombination and excision in both integrated vectors. In such embodiments, it is nonetheless preferable that the pairs of site-specific recombination sites differ between the two integration vectors (e.g., two pairs of different lox sites, two pairs of different FRT sites) to reduce the likelihood of recombination, rather than excision, between the integrated vectors. Alternatively, if the site-specific recombinase that can catalyze excision between the pair of site-specific recombination sites in the first integration vector differs from the site-specific recombinase that can catalyze excision between the pair of site-specific recombination sites in the second integration vector, then two different site-specific recombinases may be used to induce recombination and excision in both integrated vectors.

In another aspect, the invention provides methods for producing large pools of cells that have been genetically-modified (e.g., insertions or deletions causing “knock-out” mutations) at a variety of genetic targets. Specifically, in some embodiments, a variety of different types or species of guide RNAs complementary to a variety of different genetic targets can be introduced into the population of cells such that, on average, more than one target site is modified in each cell. For example, the number of guide RNA vectors delivered to each cell can, on average, be greater than 1, 2, 3, 4, 5, 6, 7, 8, 9, 10 or higher. In addition, the number of different types or species of guide RNAs delivered to the population of cells can be greater than 1, 10, 10², 10³, 10⁴ or higher. This will result in a population or pool of genetically modified cells in which most cells will be genetically-modified at more than one genetic target and in which there are many types or subsets of cells with different combinations of modified targets. For example, with 10 targets (or, more generally, X targets) and each cell being modified at exactly two different target sites, there would be 45 possible combinations of modified targets (or, more generally, X(X−1)/2), and for 10³ targets there would be 499,500. With more guide RNA vectors delivered to each cell (i.e., similar to a higher multiplicity of infection) and more types or species of guide RNA vectors, an incredibly diverse or complex pool of genetically-modified cells can be produced.

Such pools of cells with multiple genetically-modifications can be useful in screening for therapeutic targets and agents for a variety of disease, including cancer. For example, populations of cancer cells with varying genetic loci knocked-out can be introduced into animal models and subjected to treatments with known or potential therapeutics. Cancer cells which escape the treatment can be studied to determine the basis for resistance, or cells which are susceptible to the treatment can be studied to identify cancers for which the treatment is effective.

Retroviral Vectors

Retroviral vectors can be derived from any of the Alpharetroviruses, Betaretroviruses, Gammaretroviruses, Deltaretroviruses, Epsilonretroviruses, or Lentiviruses. At present, the Gammaretroviruses and the Lentiviruses have been most studied and adapted for use in genetic engineering and gene therapy, being especially important the vectors derived from human immunodeficiency virus (HIV)-1. For safety, the viruses are modified to make them replication defective and, therefore, they may be produced with the aid of packaging plasmids or packaging cell lines. Thus, common modifications included in retroviral vectors are deletion and/or inactivation of one or more of the gag, pol and end proteins which are necessary for replication.

Lentiviruses can be classified into five families (1) primate, (2) bovine, (3) ovine/caprine, (4) equine and (5) feline. Lentiviral vectors derived from primate lentiviruses are preferred in the present disclosure, although other lentiviral vectors may be used.

For brevity, the following discussion focuses on lentiviral vectors, although it will be apparent to those of skill in the art that it applies to retroviral vectors generally and that other retroviral vectors fall within the scope of the invention.

Lentiviruses have been developed as efficient delivery vectors for gene therapy and genome editing because they can integrate a significant amount of viral cDNA into the genome of a host cell and because they can infect non-dividing cells. Lentivirus particles contain two single-stranded positive sense RNA-genomes. The native lentivirus genome is approximately 10 kb long and is flanked by long terminal repeats (LTRs). A sequence located near the 5′ end of the genome, known as the Psi (Ψ) packaging element, is necessary for packaging viral RNA into capsids and, therefore, is included in the vectors of the invention. For simplicity, the Psi element is omitted from some figures but is understood to be present immediately 3′ of the 5′ LTR. Transgenes intended for integration by lentiviral vectors may be included between the 5′ Psi sequence and the 3′ LTR.

Prior to integration into a host genome, the lentiviral RNA genome may be converted into DNA by a reverse transcriptase that synthesizes a first strand of DNA from the RNA genomeA host cell DNA polymerase then synthesizes the second strand to produce a double-stranded DNA. Integration of the vector is mediated by an integrase and the LTRs. Lentiviral LTRs typically comprise about 600 nucleotides and include distinct U3, R and U5 regions.

Prior to integration, certain LTR elements are duplicated during reverse transcription. Specifically, the U3 region in the 3′ LTR region is copied and incorporated into the 5′ LTR. Thus, if part of the U3 region in the 3′ LTR is deleted, the same deletion will be duplicated into the 5′ LTR. Similarly, if a nucleotide sequence is inserted into the U3 region of the 3′ LTR (e.g., a site-specific recombination site), the same insertion will be duplicated into the 5′ LTR during reverse transcription of the viral RNA genome. Thus, after integration, such deletions/insertions will be present in both the 5′ and 3′ LTRs of the provirus.

Lentiviral vectors are produced by modifying lentiviruses such that they are replication defective but still capable of integration, have deletions of one or more loci which are not necessary for their role as a vector (e.g., deletion or inactivation of the gag, pol and env loci needed for replication), and insertion of one or more transgenes which are necessary or useful for their role as a vector for genome-editing (e.g., a Cas coding sequence, detectable markers).

In some embodiments, a single site-specific recombination site is incorporated into the U3 region of the 3′ LTR region and duplicated into the 5′ LTR region during reverse transcription. Once integrated into the host cell genome, the provirus contains one site-specific recombination site in the 5′ LTR region and the same site-specific recombination site in the 3′ LTR region. A site-specific recombinase that recognizes this pair of site-specific recombination sites can catalyze the excision of the nucleotide sequence flanked by the pair of site-specific recombination sites. In other embodiments, a pair of site-specific recombination sites are present on the lentiviral vector prior to reverse transcription and the 3′ site specific-recombination site is located upstream of the U3 region of the 3′ LTR. Therefore, in those embodiments, the 3′ site-specific recombination site will not be duplicated with the 3′ LTR during reverse transcription and integration. Non-limiting examples of single site-specific recombination sites useful in the invention include lox sites, FRT sites and Lox sites.

The CRISPR/Cas lentiviral vectors of the invention are reproduction or replication defective, but are not integration deficient. Thus, the vectors can integrate into a host genome but cannot reproduce themselves. Therefore, the vectors may be produced by transfecting the lentiviral vector with one or more plasmids that encode the viral components necessary to produce an infectious viral particle, including proteins necessary for produced viral capsids and packaging viral genomes into the capsids. A variety of such packaging systems, including packaging plasmids or packaging cell lines, are known in the art and widely available. The most commonly used systems are known as second and third generation lentiviral packaging systems.

In some embodiments, the lentiviral vector can be paired with a second generation packaging system. Such second generation lentiviral packaging systems can include a single packaging plasmid encoding the Gag, Pol, Rev, and Tat genes. The lentiviral vector of the invention will include the viral LTRs, Psi packaging signal and transgenes (e.g., Cas, detectable marker(s)). Unless an internal promoter is provided (e.g., “Promoter 1” as described above), gene expression is driven by the 5′ LTR, which is a weak promoter and may require the presence of Tat to activate expression. The envelope protein Env (usually VSV-G due to its wide infectivity) can be encoded on a third, separate, envelope plasmid. Non-limiting examples of second generation lentiviral packaging plasmids include psPAX2, pCMV delta R8.2, pCMV-dR8.2 dvpr, pCPRDEnv, pCD/NL-BH*DDD, psPAX2-D64V, and pNHP. Non-limiting examples of second generation lentiviral envelope plasmids include pMD2.G, pCMV-VSV-G, pLTR-RD114A, and pLTR-G.

In some embodiments, the lentiviral vector can be paired with a third generation packaging system. The third generation systems further improve on the safety of the second generation systems in several ways. First, the packaging plasmid is split into two plasmids: one encoding Rev and one encoding Gag and Pol. Second, Tat is eliminated from the third generation system through the addition of a chimeric 5′ LTR fused to a heterologous promoter on the transfer plasmid. Expression of the transgene(s) from this promoter is not dependent on Tat transactivation. The third generation vectors can be packaged by either a second generation or third generation packaging system. Non-limiting examples of the third generation lentiviral packaging plasmids include pRSV-Rev, and pMDLg/pRRE.

Other Vectors

In some embodiments, the sgRNA and/or site-specific recombinase transgenes are delivered by non-retroviral vectors, such as SV40 or adeno-associated virus (AAV) vectors.

One major advantage of using AAV for research is that it is replication-limited and typically not known to cause disease in humans. For these reasons, AAVs are generally contained at lower biosafety levels and elicit relatively low immunological effects in vivo. AAV can transduce both dividing and non-dividing cells with a low immune response and low toxicity. Although recombinant AAV does not integrate into the host genome, transgene expression can be long-lived. The utility of AAV is currently limited by its small packaging capacity (˜4.5 kb including inverted terminal repeats (ITRs)), though there is a great deal of interest and effort directed toward expanding this capacity. The small (4.8 kb) ssDNA AAV genome consists of two open reading frames, Rep and Cap, flanked by two 145 base ITRs. These ITRs base pair to allow for synthesis of the complementary DNA strand. Rep and Cap are translated to produce multiple distinct proteins (Rep78, Rep68, Rep52, Rep40—required for the AAV life cycle; VP1, VP2, VP3—capsid proteins). When constructing an AAV transfer vector, the transgene is placed between the two ITRs, and Rep and Cap are supplied in trans. In addition to Rep and Cap, AAV requires a helper plasmid containing genes from adenovirus. These genes (E4, E2a and VA) mediate AAV replication. The transfer plasmid, Rep/Cap, and the helper plasmid are commonly transfected into cells such as HEK293 cells, which contain the adenovirus gene E1+, to produce infectious AAV particles. Rep/Cap and the adenovirus helper genes can also be combined into a single plasmid. Eleven serotypes of AAV have thus far been identified, with the best characterized and most commonly used being AAV2. These serotypes differ in their tropism, or the types of cells they infect, making AAV a very useful system for preferentially transducing specific cell types.

Promoters

Exogenous promoters useful in the invention include eukaryotic promoters as well as viral promoters that function in eukaryotic host cells, and particularly human and other mammalian host cells.

A promoter can be a constitutively active promoter (i.e., a promoter that is constitutively or constantly in an active/“ON” state); an inducible promoter (i.e., a promoter that is active/“ON” or inactive/“OFF” depending upon an external stimulus (e.g., the presence of a particular temperature, compound, or protein); a spatially restricted promoter (e.g., tissue specific promoter, cell type specific promoter, etc.); or temporally restricted promoter (i.e., the promoter is in the “ON” state or “OFF” state during specific stages of embryonic development or during specific stages of a biological process (e.g., hair follicle cycle in mice)). In some embodiments, a constitutive promoter is preferred for CRISPR/Cas and/or sgRNA transgenes.

Suitable promoters can be derived from viruses, prokaryotic or eukaryotic organisms, and can be used to drive expression by any RNA polymerase (e.g., pol I, pol II, pol II I). Exemplary promoters include, but are not limited to the SV40 early and late gene promoters, mouse mammary tumor virus long terminal repeat (LTR) promoter; mouse metallothionein-1 gene promoter; adenovirus major late promoter (Ad MLP); a herpes simplex virus (HSV) thymidine kinase gene promoter, a cytomegalovirus (CMV) promoter such as the CMV immediate early promoter region (CMVI E), a rous sarcoma virus (RSV) promoter, a human U6 small nuclear promoter (U6) (Miyagishi et al. (2002), Nature Biotechnology 20: 497-500), an enhanced U6 promoter (e.g., Xia et al. (2003), Nucleic Acids Res. 31(7)), a human H1 promoter, an EF1α promoter, and the like.

In some embodiments, the promoter is a constitutive promoter. Constitutive promoters direct expression that is largely, if not entirely, independent of environmental and developmental factors. As their expression is normally not conditioned by endogenous factors, constitutive promoters are usually active across species and even across kingdoms. Non-limiting examples of constitutive promoters are CMV, EF|α. SV40, PGK1, Ubc, human beta actin, CAG, Ac5, Polyhedrin, TEF1m GDS, CaMV355, Ubi, H1, and U6.

Preferably, the transgenes of the CRISPR/Cas vector are under the control of constitutive promoters, although inducible promoters can be used.

In some embodiments, the promoter is an inducible promoter. Inducible promoters are only active under specific circumstances. Non-limiting examples of factors that can activate an inducible promoter include the presence of certain chemical compounds (i.e., inducers) or the absence of certain chemical compounds (i.e., repressors), temperature, light, etc. Non-limiting examples of inducible promoters are TRE, GAL1.10, AlcR, Hsp-70, Hsp-90, FixK2, T7 RNA polymerase promoter, T3 RNA polymerase promoter, Isopropyl-beta-D-thiogalactopyranoside (IPTG)-regulated promoter, lactose induced promoter, heat shock promoters, tetracycline-regulated promoters, steroid-regulated promoters, metal-regulated promoters, estrogen receptor-regulated promoters, etc.

In some embodiments, the promoter is a tissue-specific promoter. Tissue-specific promoters direct the expression of a gene in a specific tissue or at certain developmental state. A transgene operably linked to a tissue-specific promoter can be expressed in the specific tissue where the promoter is active. Non-limiting examples of tissue specific promoters include B29 promoter for expression of transgenes in B cells; CD14 promoter for expression of a transgene in monocytic cells; desmin promoter for expression of transgene in muscle cells; elastase-1 promoter for expression of transgene in pancreatic cells; endoglin promoter for expression of transgene in endothelial cells, and GFAP promoter for expression of transgene in neuron cells.

Spacers

A spacer, as used herein, refers to a nucleotide sequence positioned between coding sequences in a polycistronic locus or polycistronic mRNA to facilitate the translation or processing of the two coding sequences into two separate proteins. Non-limiting examples of a spacer are internal ribosome entry sites (IRES), self-cleaving peptide coding sequences, and nucleotide sequences encoding an endogenous protease cleavage site.

In some embodiments, the spacer is an IRES. An IRES, as used herein, refers to a DNA sequence that, once transcribed into mRNA, allows for initiation of translation from an internal region of the mRNA. Translation in eukaryotes usually begins at the 5′ cap of the mRNA so that only a single translation event occurs for each mRNA. An IRES, however, can initiate translation independent of the 5′ cap and acts as another ribosome recruitment site, thereby resulting in co-expression of two proteins from a single mRNA.

In some embodiments, the spacer encodes a self-cleaving peptide, including without limitation 2A, E2A, F2A, P2A and T2A self-cleaving peptides. A self-cleaving 2A peptide, as used herein, refers to a short oligopeptide (usually 19-22 amino acids) located between two proteins in some members of the picornavirus family3. The 2A self-cleaving peptide can undergo self-cleavage to generate mature proteins by a translational effect that is known as “stop-go” or “stop-carry” (Wang et al. (2015), Nature Scientific Reports 5:16237). The term “self-cleaving” is not entirely accurate, as these peptides are thought to function by making the ribosome skip the synthesis of a peptide bond at the C-terminus of a 2A element, leading to separation between the end of the 2A sequence and the next peptide downstream. The “cleavage” occurs between the Glycine and Proline residues found on the C-terminus meaning the upstream cistron will have a few additional residues added to the end, while the downstream cistron will start with the Proline.

In some embodiments, the spacer encodes for a cleavage site for protease that is endogenous to the host cell. Non-limiting examples of proteases are trypsin, elastase, matrix metalloproteinases (MMPs), and pepsin.

Other DNA Regulatory Elements

In some embodiments, any of the vectors of the invention can comprise one or more individual restriction endonuclease recognition sequences or one or more multiple cloning sites. These sites can be located upstream and/or downstream of one or more sequence elements of one or more vectors.

In come embodiments, any of the vectors of the invention can comprise an enhancer sequence such as a Woodchuck Hepatitis Virus Post-transcriptional Regulatory Element (WPRE) sequence. WPRE sequences are commonly used in molecular biology to increase expression of genes delivered by viral vectors. WPRE is a tripartite regulatory element and usually is positioned at the 3′ UTR of a mammalian expression cassette to significantly increase mRNA stability and protein yield.

In some embodiments, a guide RNA vector comprises an insertion site upstream of a tracr mate sequence, and optionally downstream of a regulatory element operably linked to the tracr mate sequence, such that following insertion of a guide sequence into the insertion site and upon expression, the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences can comprise two or more copies of a single guide sequence, two or more different guide sequences, or combinations of these. When multiple different guide sequences are used, a single expression construct can be used to target CRISPR activity to multiple different, corresponding target sequences within a cell. For example, a single vector can comprise about 1, 2, 3, 4, 5, 6, 7, 8, 9, 10, 15, 20 or more guide sequences.

CRISPR/Cas9 Systems

The present disclosure, at least in part, relates to using CRISPR/Cas system for introducing genetic modification to a population of cells. In some embodiments, the cells are cancer cells. In some embodiments, the genetic modification is a knock-out of an endogenous gene. In other embodiments, the genetic modification is a knock-in of an exogenous gene.

In some aspects, the first integration vector (the “Cas vector”) comprises a promoter operably linked to a first nucleic acid sequence comprising a first promoter operably linked to a Cas protein coding sequence encoding the open reading frame of a Cas protein. The Cas protein, is integrated into the host cell genome for stable expression.

In general, CRISPRs (Clustered Regularly Inter spaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al. (1987), J. Bacteriol., 169:5429-5433; and Nakata et al. (1989), J. Bacteriol., 171:3553-3556), and associated genes. Similar interspersed SSRs have been identified in Haloferax mediterranei, Streptococcus pyogenes, Anabaena, and Mycobacterium tuberculosis (See, Groenen et al. (1993), Mol. Microbiol., 10:1057-1065; Hoe et al. (1999), Emerg. Infect. Dis., 5:254-263; Masepohl et al. (1996), Biochim. Biophys. Acta 1307:26-30; and Mojica et al. (1995), Mol. Microbiol., 17:85-93. The CRISPR loci typically differ from other SSRs by the structure of the repeats, which have been termed short regularly spaced repeats (SRSRs) (Janssen et al. (2002), OMICS J. Integ. Biol. 6:23 33; and Mojica et al. (2000), Mol. Microbiol. 36:244-246).

In general, the repeats are short elements with a substantially constant length (Mojica et al. (2000), supra). Although the repeat sequences are highly conserved between strains, the number of interspersed repeats and the sequences of the spacer regions typically differ from strain to strain (van Embden et al. (2000), J. Bacteriol. 182:2393-2401. CRISPR loci have been identified in more than 40 prokaryotes (see, e.g., Jansen et al. (2002), Mol. Micro biol. 43:1565-1575) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter; Mycoplasma, Fusobacterium, Azarcus, Chromobacterium, Neisseria, Nitrosomonas, Desulfovibrio, Geobacter, Myxococcus, Campylobacter; Wolinella, Acinetobacter, Erwinia, Escherichia, Legionella, Methylococcus, Pasteurella, Photobacterium, Salmonella, Xanthomonas, Yersinia, Treponema, and Thermotoga.

In general, a “CRISPR system” refers collectively to coding sequences and other elements involved in the expression of CRISPR-associated (“Cas”) genes, including sequences encoding a Cas gene, a tracr (transactivating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence, or other sequences and transcripts from a CRISPR locus. In some embodiments, one or more elements of a CRISPR system is derived from a type I, type II, or type III CRISPR system. In some embodiments, an element of a CRISPR system is derived from a particular organism comprising an endogenous CRISPR system, such as Streptococcus pyogenes. In general, a CRISPR system is characterized by elements that promote the formation of a CRISPR complex at the site of a target sequence. In the context of formation of a CRISPR complex, “target sequence” refers to a sequence to which a guide RNA sequence is designed to have complementarity, where hybridization between a target sequence and a guide RNA sequence promotes the formation of a CRISPR complex. Full complementarity is not required, provided there is sufficient complementarity to cause hybridization and promote formation of a CRISPR complex. A target sequence can comprise any polynucleotide, such as DNA or RNA polynucleotides.

As used herein, the term “Cas protein” refers to a CRISPR associated protein, or analog or variant thereof, and embraces any naturally occurring Cas from any organism, any naturally-occurring Cas, any Cas homolog, ortholog, or paralog from any organism, and any analog of a Cas, naturally-occurring or engineered (e.g., a naturally-occurring or engineered Cas9). The term “Cas” is not meant to be limiting and may be referred to as a “Cas or an analog thereof.”

In some embodiments, proteins comprising Cas or fragments thereof are referred to as “Cas analogs.” A Cas analog shares homology to Cas, or a fragment thereof. Cas analogs include functional fragments of Cas. For example, a Cas9 analog is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 analog may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a wild type Cas9. In some embodiments, the Cas9 analog comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

Non-limiting examples of Cas proteins include S. pyogenes Cas9 (also known as SpCas9, Csn1 and CSX12), Cpf1, Cas9 nickase, nuclease-inactive Cas9 (also known as dead Cas9), S. aureus Cas9 (SaCas9), Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, CSm3, Csm4, Csm5, Csm6, Cmr1, Cimr3, Cimra, CimrS, Cmré, Csb1, Csb2, Csb3, CSX17, CSX14, CSX10, CSX16, CsaX, CSX3, CSX1, CSX15, Csf1, Csf2, Csf3, Csf4, C2c1, C2c2 (Cas13a), C2c3 (Cas12c), GeoCas9, CjCas9, Cas12a, Cas12b, Cas12g, Cas12h, Cas12i, Cas13b, Cas13c, Cas13d, Cas14, Csn2, Argonaute, evolved Cas9 domains (xCas9) and circularly permuted Cas9 proteins such as CP1012, CP1028, CP1041, CP1249, and CP1300. These enzymes are known in the art and their nucleic acid and amino acid sequences are publicly available; for example, the amino acid sequence of S. pyogenes Cas9 protein can be found in the SwissProt database under accession number Q99ZW2.

In some embodiments the Cas protein is Cas9, and can be Cas9 from S. pyogenes, S. aureus or S. pneumoniae. In some embodiments, the Cas protein directs cleavage of one or both strands at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In other embodiments, a nucleotide sequence encodes for a Cas9 analog. A Cas9 analog, as used herein, refers to other natural occurring or engineered Cas9 that is capable of double-strand DNA cleavage at the site targeted by sgRNA. A non-limiting example of a reduced-size Cas9 analog includes Cpf1 and SaCas9. Cpf1, as used herein, refers to a type II CRIPSR enzyme. Cpf1 mediates robust DNA interference with features distinct from Cas9. Cpf1 is a single RNA-guided endonuclease lacking tracrRNA. Cpf1-mediates DNA cleavage creates DSBs with a short 3′ overhang. Cpf1 's staggered cleavage pattern opens up the possibility of directional gene transfer, analogous to traditional restriction enzyme cloning, which may increase the efficiency of gene editing Like the Cas9 variants and orthologs described above, Cpf1 also expands the range of sites that can be targeted by CRISPR to AT-rich regions or AT-rich genomes that lack the NGG PAM sites favored by SpCas9. For instance, the Cas9 protein may comprise a S. pyogenes Cas9-NG variant that recognizes an expanded PAM, i.e., most NG PAM sites. This variant is disclosed in Nishimasu et al., Science 361, 1259-1262 (2018), incorporated herein by reference. In other embodiments, the cas9 protein may comprise a Cas9 analog that has been evolved to recognize an expanded PAM, as recently reported in Hu et al., Nature, 556(7699):57-63 (2018) and International Application No. PCT/US2019/47996, filed Aug. 23, 2019, each of which is incorporated by reference herein. Exemplary evolved Cas9 variants having expanded PAM specificities include xCas9 (3.6) and xCas9 (3.7).

In some embodiments, the Cas9 analog is SaCas9. An SaCas9, as used herein, refers to a Cas9 protein derived from Staphylococcus aureus. SaCas9 is ˜1 kilobase shorter than SpCas9, which renders it more versatile to be packaged into various vector systems (e.g., AAV vectors, lentiviral vectors). Similar to SpCas9, the SaCas9 endonuclease is capable of modifying target genes in mammalian cells in vitro and in mice in vivo. In some embodiments, the Cas protein is is codon optimized for expression in particular cells, such as eukaryotic cells. The eukaryotic cells can be those of or derived from a particular organism, such as a mammal, including but not limited to human, non-human primate, mouse, rat, rabbit dog. In some embodiments, the Cas9 protein is an engineered Cas9 that is capable of recognizing non-NGG PAM sequences.

In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell Biol., 2015 Nov. 5; 60(3): 385-397, which is incorporated herein by reference. In some embodiments, a napDNAbp domain may comprise a CasX (now referred to as Cas12e) or CasY (now referred to as Cas12d) omain, which have been described in, for example, Burstein et al., “New CRISPR-Cas systems from uncultivated microbes.” Cell Res. 2017 Feb. 21. doi: 10.1038/cr.2017.21, and Liu et al., “CasX enzymes comprise a distinct family of RNA-guided genome editors,” Nature. 2019; 566(7743):218-223, each of which is incorporated herein by reference. In other embodiments, the Cas protein provided herein may be a CjCas9, Cas12a, Cas12b, Cas12g, Cas12h, Cas12i, Cas13b, Cas13c, Cas13d, Cas14, Csn2, and GeoCas9. CjCas9 is described and characterized in Kim et al., Nat Commun. 2017; 8:14500 and Dugar et al., Molecular Cell 2018; 69:893-905, incorporated herein by reference. GeoCas9 is described and characterized in Harrington et al. Nat Commun. 2017; 8(1):1424 and International Publication No. PCT/US2019/58678, filed Oct. 29, 2019, each of incorporated herein by reference. The Cas12a, Cas12b, Cas12g, Cas12h and Cas12i proteins are described and characterized in, e.g., Yan et al., Science, 2019; 363(6422): 88-91, Murugan et al. The Revolution Continues: Newly Discovered Systems Expand the CRISPR-Cas Toolkit, Molecular Cell 2017; 68(1):15-25, each of which are incorporated herein by reference. Cas14 is characterized and described in Harrington et al. Science 2018; 362(6416):839-842, incorporated herein by reference. Cas13b, Cas13c and Cas13d are described and characterized in Smargon et al., Molecular Cell 2017, Cox et al., Science 2017, and Yan et al. Molecular Cell 70, 327-339.e5 (2018), each of which are incorporated herein by reference. Csn2 is described and characterized in Koo Y., Jung D. K., and Bae E. PloS One. 2012; 7:e33401, incorporated herein by reference.

In some embodiments, the Cas protein is mutated with respect to a corresponding wild-type enzyme such that the mutated Cas protein lacks the ability to cleave one or both strands of a target polynucleotide containing a target sequence. In particular embodiments, an aspartate-to-alanine substitution (D10A) in the RuvC1 catalytic domain of S. pyogenes Cas9 converts Cas9 from a nuclease that cleaves both strands to a nickase that nicks the targeted strand, or the strand that is complementary to the sgRNA. A histidine-to-alanine substitution (H840A) in the HNH catalytic domain of S. pyogenes Cas9 generates a nick on the strand that is displaced by the sgRNA during strand invasion, also referred to herein as the non-edited strand. The single catalytically active nuclease site of the nCas9 leaves a nick in the non-edited strand, which will direct mismatch repair machinery to read (rather than remove) a mutated sequence in the target gene during repair. Other examples of mutations that render Cas9 a nickase include, without limitation, N854A and N863A in SpCas9, and corresponding mutations in other wild-type Cas9 proteins or analogs thereof. Reference is made to U.S. Pat. No. 8,945,839, which is incorporated herein by reference.

In nature, CRISPR clusters are transcribed and processed into CRISPR RNA (crRNA). In certain types of CRISPR systems (e.g., type II CRISPR systems), correct processing of pre-crRNA may require a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage may require protein and both RNAs. However, single guide RNAs (“sgRNA”, or simply “gRNA”) can be engineered so as to incorporate embodiments of both the crRNA and tracrRNA into a single RNA species—the guide RNA. See, e.g., Jinek M., et al., Science 337:816-821 (2012), which is incorporated herein by reference.

In general, a guide RNA is any polynucleotide sequence having sufficient complementarity with a target polynucleotide sequence to hybridize with the target sequence and direct sequence-specific binding of a CRISPR complex (e.g., a Cas9) to the target sequence. In some embodiments, the degree of complementarity between guide RNA and its corresponding target sequence, when optimally aligned using a suitable alignment algorithm, is about or more than about 50%, 60%, 75%, 80%, 85%, 90%, 95%, 97.5%, 99%, or more. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW. Clustal X, BLAT, Novoalign (Novocraft Technologies, ELAND (Illumina, San Diego, Calif.), SOAP (available at Soap.genomics.org.cn), and Maq (available at maq.Sourceforge.net).

In some embodiments, the guide sequence of the sgRNA is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. The guide sequence is typically 20 nucleotides long. See U.S. Publication No. 2015/0166981, published Jun. 18, 2015, which is incorporated by reference herein. In some embodiments, the sgRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a sequence in a target gene.

The guide sequence of the sgRNA is linked to a tracr mate (also known as a “backbone”) sequence which in turn hybridizes to a tracr sequence. In some embodiments, the guide RNAs for use in accordance with the disclosed methods comprise a backbone structure that is recognized by an S. pyogenes Cas9 protein.

In some embodiments, the sgRNA is delivered into the cells as single stranded RNA. In some embodiments, the sgRNA is delivered into the cells on an expression vector. In some embodiments, the sgRNA is delivered into the cells on the first integration vector (Cas vector). In other embodiments, the sgRNA is delivered into the cells on a second integration vector (the “guide RNA vector”).

Selectable or Detectable Markers

In some embodiments, the first integration vector (or “Cas vector”) and/or second integration vector (or “sgRNA vector”) further comprises one or more detectable markers.

A detectable marker, as used herein, refers to an exogenous gene introduced into the host cell by a vector of the invention that confers a trait suitable for artificial selection or detection. Non-limiting examples for selectable markers include fluorescent proteins, antibiotic resistance genes, cell surface markers and enzymes.

In some embodiments, the detectable marker is a fluorescent protein. Non-limiting examples of fluorescent proteins are Green Fluorescent Protein (GFP) or Enhanced Green Fluorescent Protein (EGFP), Red Fluorescent Protein (RFP), Yellow Fluorescent Protein (YFP), Cyan Fluorescent protein (CFP), Blue Fluorescent Protein (BFP), mCherry, and tdTomato. The presences of the fluorescence protein can be detected by flow cytometric analysis.

In some embodiments, the detectable marker is an antibiotic resistance gene. Non-limiting examples of antibiotic resistance genes are the bls gene, hph gene, sh ble gene, or neo gene. In some embodiments, the selectable marker is the bls gene, and cells that express the bls gene are resistant to blasticidin. In another embodiment, the selectable marker is the hph gene, and cells that express the hph gene are resistant to hygromycin B. In yet another embodiment, the selectable marker is the sh ble gene, and the cells that express the sh ble gene are resistant to zeocin and phleomycin. In yet another embodiment, the selectable marker is the neo gene and the cells that express the neo gene are resistant to geneticin.

In some embodiments, the detectable marker is a cell surface marker. The presence of the cell surface marker can be detected by staining the cells with an antibody that is specific to the cell surface marker and that is conjugated with a fluorophore.

In some embodiments, the detectable marker is an enzyme. Non-limiting examples of an enzymes useful as detectable markers include luciferase, horseradish peroxidase (HRP) and beta-galactosidase. The expression of these enzyme can be detected by adding the corresponding substrate into the cells and detecting the resulting bioluminescent or chromogenic product.

In some embodiments, the detectable markers on the Cas vector and the guide RNA vector are detected by different means (e.g., color, fluorescence, resistance).

Site-Specific Recombinases and Recombination Sites

In some aspects, the present disclosure provides recombinogenic vectors comprising pairs of site-specific recombination sites flanking the coding sequences of one or more proteins that may be immunogenic to the host cell. As described above, in some embodiments, both of a pair of sites are present before integration of the vector, and in some embodiments both of a pair of sites are present only after reverse transcription duplicates a 3′ LTR including one of the sites.

Site-specific recombination sites, as used herein, refer to DNA sequences that are typically between 30 and 200 nucleotides in length and consist of two motifs with a partial inverted-repeat symmetry, to which a site-specific recombinase binds and mediates recombination. Site-specific recombinases, as used herein, refers to a group of enzymes that catalyze directionally sensitive DNA exchange reactions between target site sequences that are specific to each recombinase. Non-limiting examples of site specific recombinase-site specific recombination sites pairs include Cre-Lox, Flp-FRT, ΦC31-attP/attB, and Dre-Rox. Thus, in some embodiments, the recombinase is Cre, Flp, ΦC31 or Dre, and in some embodiments, the site-specific recombination sites are lox, FRT, attP/attB and rox, respectively.

In some embodiments, the site-specific recombination sites are lox sites. Lox sites are typically about 34 base pairs and consist of two palindromic regions of about 13 bp and an intervening non-palindromic spacer of about 8 bp that determines the orientation of the site. When two lox sites are oriented in the same direction, the site-specific recombinase Cre excises the DNA flanked by the lox sites, leaving a single lox site behind.

Differences in palindromic or spacer regions of lox sites, either naturally-occurring or randomly mutated, can confer specificity to Cre recognition. Non-limiting examples of mutated lox sites are loxP511, lox2272, loxΔ86, loxΔ117, loxC2, loxP2, loxP3, loxP23, loxB, loxL and loxR, all of which are known in the art. In some embodiments, the lox sites are loxP sites. In some embodiments, the lox sites are mutated lox sites. In some embodiments, the mutated lox sites are lox2272. In other embodiments, the mutated lox sites are lox5171. The Lox-Cre system is disclosed in further detail in Sauer, B. (1987), Mol Cell Biol. 7 (6): 2087-2096; Tsien, Joe Z. (2016). Frontiers in Genetics. 7: 19; Shakes et al., Nucleic Acids Res. 2005; 33(13): e118; R H Hoess, M Ziese, & N Sternberg, PNAS Jun. 1, 1982, 79(11): 3398-3402; Michel G, et al., Mol Ther. 2010; 18(10):1814-21; and U.S. Pat. Nos. 6,828,093 and 7,179,644, each of which is incorporated herein by reference.

In some embodiments, the site-specific recombination sites are FRT sites. The FRT sites are about 34 bp and consist of two palindromic regions of about 13 bp and an intervening non-palindromic core region of about 8 bp that determines the orientation of the site. Several variant FRT sites exist, but recombination can usually occur only between two identical FRTs and not among non-identical or “heterospecific” FRTs. When two FRT sites are oriented in the same direction, the site-specific recombinase Flp can excise the DNA flanked by the FRT sites, leaving a single FRT site behind. See Schubeler D, Maass K & Bode J, Biochemistry. 1998 Aug. 25; 37(34):11907-14, incorporated herein by reference.

In some embodiments, the site-specific recombination sites are attL and attR sites. The attL and attR sites are recognized by the ΦC31 integrase, a site-specific bacteriophage recombinase. See Pokhiliko et al., Nucleic Acids Res. 2016; 44(15): 7360-7372, incorporated herein by reference.

In some embodiments, the site-specific recombination sites are rox sites. The rox sites are recognized by Dre recombinase. Dre recombinase is a bacteriophage-derived tyrosine recombinase that recognizes a pair of identical rox sites and leaves behind a single rox site after recombination. See Anastassiadis K et al., Disease Models & Mechanisms 2009 2: 508-515, incorporated herein by reference.

In some embodiments of the first integration vector (or “Cas vector”), at least the coding sequence encoding the Cas protein is flanked by the site-specific recombination sites. In some embodiments of the first integration vector, the coding sequences encoding the Cas protein and at least one detectable marker are flanked by the site-specific recombination sites. In some embodiments, the site-specific recombination sites also flank at least some other components, such as promoters, spacers, enhancers, multiple cloning sites, etc.

In some embodiments of the second integration vector (or “guide RNA vector”), the coding sequence of at least one detectable marker is flanked by the site-specific recombination sites. In some embodiments of the second integration vector, the coding sequence of at least one detectable marker and the sgRNA sequence are flanked by the site-specific recombination sites. In some embodiments, the site-specific recombination sites also flank at least some other components, such as promoters, spacers, enhancers, multiple cloning sites, etc.

In order to excise the nucleotide sequences flanked by the site specific recombination sites, a site-specific recombinase that catalyzes the recombination between the site-specific recombination sites needs to be delivered the cells. In some embodiments, the recombinase is delivered as a protein. In some embodiments, the recombinase is delivered by a delivery vector. In some embodiments, the recombinase is delivered by an expression vector. In some embodiments, the recombinase is delivered by AAV vector. In other embodiments, the recombinase is delivered by an integrase deficient lentiviral vector.

Non-limiting examples of the various embodiments of the vectors for the delivery of Cas protein are shown in FIGS. 1A-1Y. Non-limiting examples of the various embodiments of the vectors for the delivery of sgRNA are shown in FIGS. 2A-2R.

Kits for Generating Genetically Modified Cells

The present disclosure also provides recombinogenic CRISPR/Cas system vectors and kits for use in making the genetically-modified cells and pools of genetically-modified cells as described herein.

Such a kit can include one or more containers each containing vectors and reagents for use in introducing the knock-in and/or knock-out modifications into cells, such as the recombinase for catalyzing the excision of one or more CRISPR/Cas components. For example, the kit can contain one or more components of a gene editing system for making one or more knock-out modifications as those described herein. Alternatively or in addition, the kit can comprise one or more exogenous nucleic acids for expressing exogenous genes as also described herein and reagents for delivering the exogenous nucleic acids into host cells. Such a kit can further include instructions for making the desired modifications to host cells.

The instructions relating to the use of the vectors and reagents comprising such as described herein generally include information as to dosage, schedule, and method of introducing the vectors. The containers can be unit doses, bulk packages (e.g., multi-dose packages) or sub-unit doses. Instructions supplied in the kits of the disclosure are typically written instructions on a label or package insert.

The kits provided herein may be comprised within suitable packaging. Suitable packaging includes, but is not limited to, vials, bottles, jars, flexible packaging, and the like. Also contemplated are packages for use in combination with a specific device, such as an electroporator. Kits optionally can provide additional components such as buffers and interpretive information. Normally, the kit comprises a container and a label or package insert(s) on or associated with the container. In some embodiments, the disclosure provides articles of manufacture comprising contents of the kits described above.

### EXAMPLES

### Example 1: Stable Expression of CRISPR-Cas9 in Tumor Cell Lines Manifest Enhanced Immunogenicity that Causes Tumor Rejection

To demonstrate the immunogenicity effects caused by overexpression of Cas9 and sgRNA components after thei integration into host cells, lentivirus generated using classical lentiviral vectors were used to stably transduce cancer cells lines to express S. pyogenes Cas9 in CT26, D4m3a and KPC cell line (herein Cas9 virus) or sgRNA in CT26 and D4m3a cell lines (herein sgRNA virus).

Cas9 virus and sgRNA virus were generated using the standard procedure for lentivirus production as described below: 18×10⁶HEK293 cells were seeded in 25 ml of MEF media into 15 cm petri dishes (Corning). Eighteen hours later, media was replaced with warm MEF media containing plasmocin (Invivogen) at 1.25 ng/mL. For each plate, 1.8 ml of OptiMEM was mixed with 4.5 μg of pMD2.G (Addgene), 13.5 μg psPAX2 (Addgene), 18 μg of the corresponding lentiviral vector expressing either Cas9 or sgRNA and 108 pt of polyethyenimine (PEI). PEI/DNA mix was incubated for 7 min at room temperature prior to transfection. Sixteen hours post-transfection, media was replaced with fresh MEF. Virus-containing media was harvested 48 h later, centrifuged for 5 minutes at 1000 rpm and filtered through a 0.45 μM membrane to remove cell debris. Aliquots were then frozen and stored at −80° C.

Cancer cell lines were transduced with the resulting lentivirus to stably express spCas9 or sgRNA. 5×10⁴-2×10⁵ cells were plated in 12-well plate in 500 uL of complete media and 500 uL of Cas9 virus-containing media, plasmocin (1.25 ng/mL) and polybrene (5 m/mL, Sigma Aldrich).

The effect of over expressing CRISPR components in tumor cell immunogenicity was evaluated by in vivo tumor experiments. Cells were harvested and re-suspended in Hanks Balanced Salt Solution (Gibco); 1.0×10⁶ tumor cells were subcutaneously injected into the right flank of the mice. Measurements were taken manually by collecting the longest dimension (length) and the longest perpendicular dimension (width); tumor volume was calculated as: (L×W2)/2. Tumors were measured every three days beginning on day 6 after challenge until endpoint (2 cm in length). In some experiments, CT26 or KPC tumor-bearing mice received 100 μg of anti-PD-1 monoclonal rat anti-mouse antibodies (clone 29F. 1A12, BioXcell) by intraperitoneal injection at days 6, 9 and 12 after tumor inoculation. Mice inoculated with D4m3 tumor cells were treated with 50 μg of anti-PD-1 at days 9 and 12.

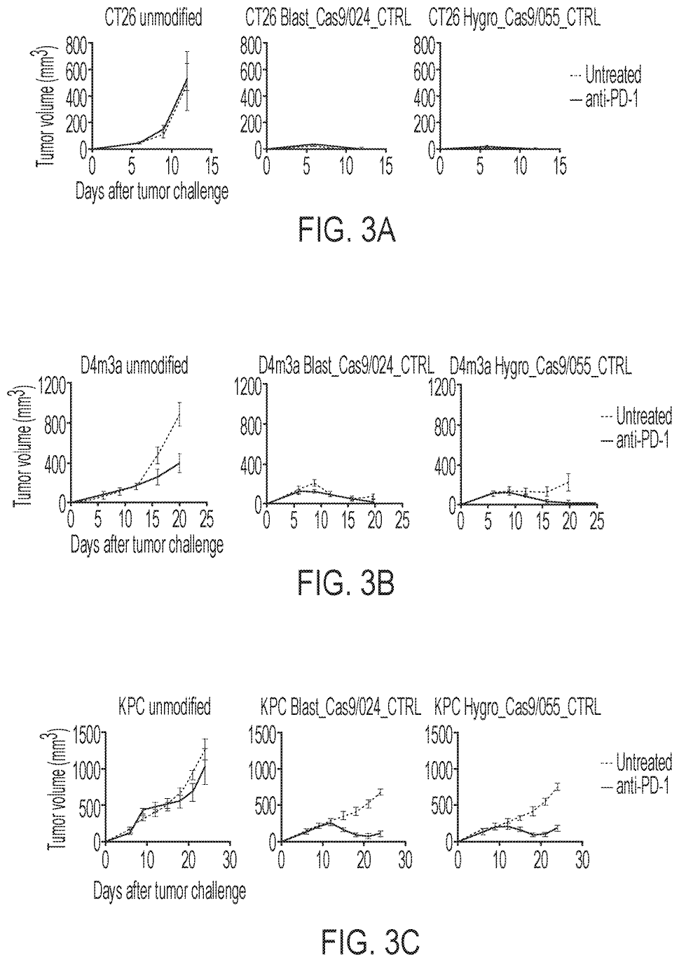

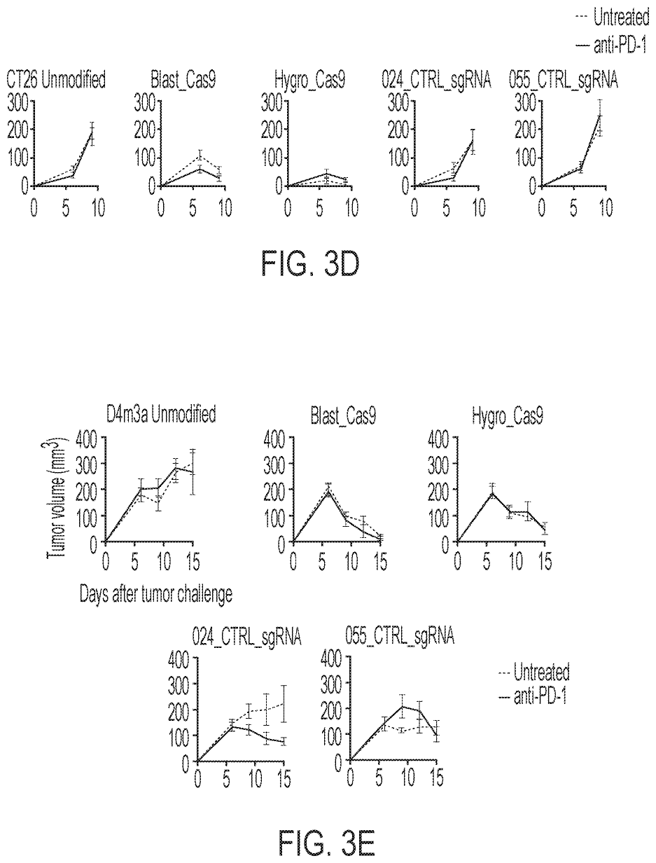

Tumor growth curves from mice challenged with CT26 (FIGS. 3A, 3D), D4m3a (FIGS. 3B, 3E) or KPC (FIG. 3C) tumor cell lines treated (solid lines) or not (dotted lines) with anti-PD-1 blocking antibodies. Stable expression of CRISPR components in tumor cells (middle and right panels) induces either tumor rejection (FIGS. 3A, 3B) or exaggerated responses to immunotherapy compared to unmodified cells (left graphs). Both Cas9 and/or sgRNA vector components cause these effects either alone (FIGS. 3D, 3E) or in combination (FIGS. 3A, 3B, 3C).

### Example 2: New Vectors Achieve Optimal Cas9 and sgRNA Expression and Genome Editing

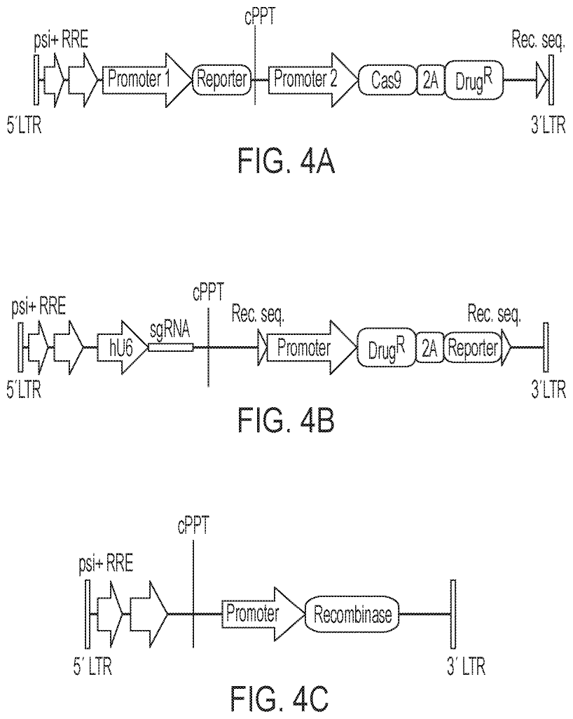

Novel methods for restoring normal cellular behavior after CRISPR-Cas9 mediated genome editing is necessary for further cancer immunology research using the genome edited cells. Here, new vector strategies for optimal Cas9 and sgRNA expression and the excision of CRISPR components after successful genome editing events were devised. FIGS. 4A-4C show schematic presentations of vectors needed to achieve optimal Cas9 and sgRNA expression for genome editing as well as the removal of CRISPR components later on. FIG. 4A is a lentiviral vector encoding (i) a reporter gene driven by promoter 1; (ii) Cas9 and a drug resistant gene driven by promoter 2; (iii) a 2A peptide located between the Cas9 and the selection gene; (iii) site specific recombination sites flanking all of the components in (i), (ii) and (iii). FIG. 4B is a lentiviral vector encoding (i) a sgRNA driven by hU6 promoter; (ii) a drug resistant gene and a reporter gene driven by another promoter; (iii) a 2A peptide located between the drug resistant gene and the reporter gene; (iv) site specific recombination sites flanking the vector components of (ii) and (iii). FIG. 4C is an integrase deficient lentiviral vector encoding a recombinase driven by a promoter.

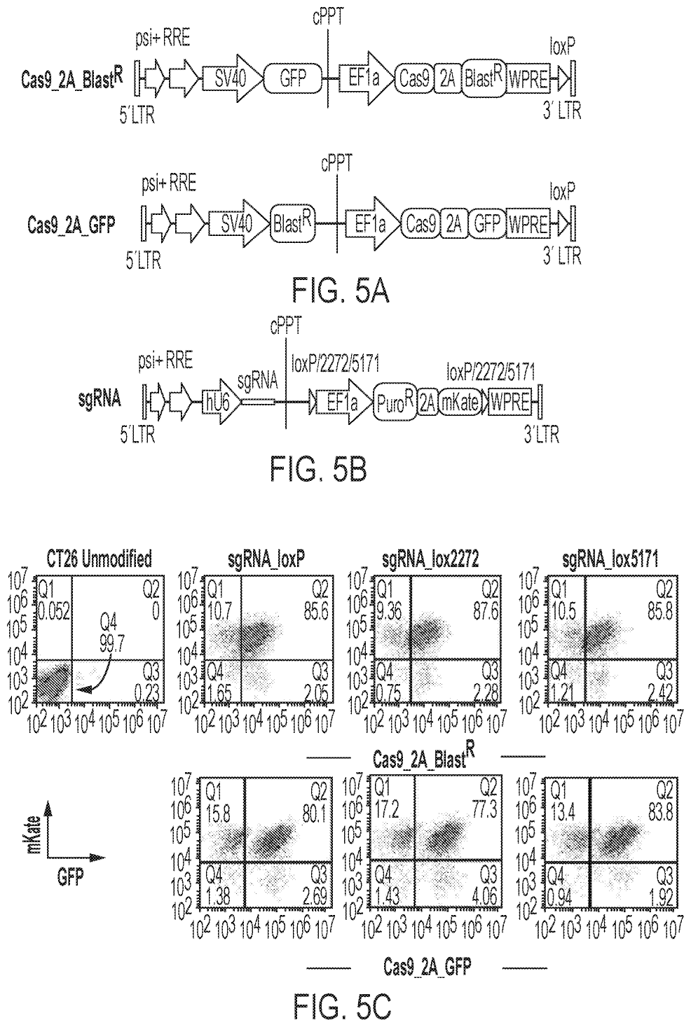

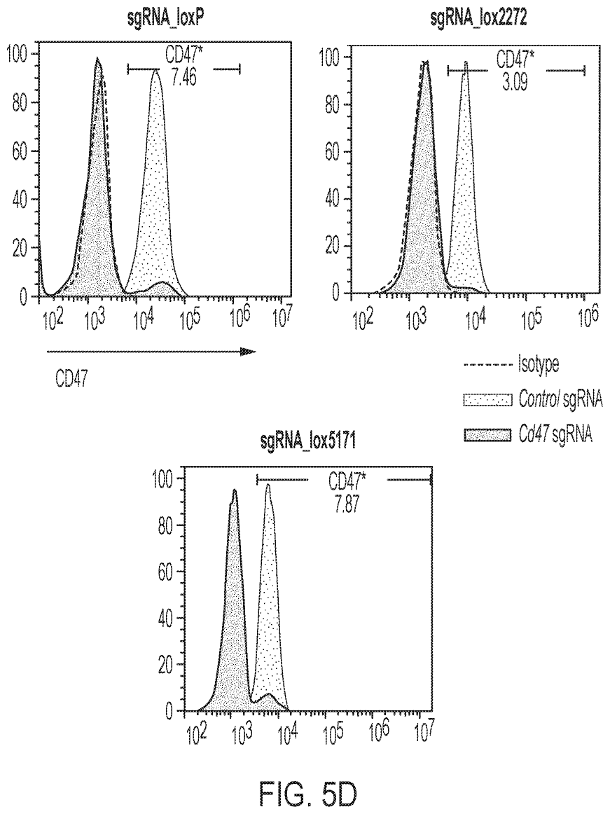

Lentiviral vectors were designed based on the scheme in FIG. 5 and the expression of Cas9 and sgRNA was confirmed by the expression of the respective reporter gene by FACS. FIG. 5A shows two different schematic illustration of the lentiviral vectors encoding Cas9. The Cas9_2A_Blast® vector is a lentiviral vector encoding (i) a GFP gene driven by SV40 promoter; (ii) Cas9 and a Blasticidin resistant gene driven by EF1α promoter; (iii) a 2A peptide located between the Cas9 and the Blasticidin resistant gene; (iv) LoxP sites flanking all of the components in (i), (ii) and (iii). The Cas9_2A_GFP vector is a lentiviral vector encoding (i) a blasticidin resistant gene driven by SV40 promoter; (ii) Cas9 and a GFP gene driven by EF1α promoter; (iii) a 2A peptide located between the Cas9 and the GFP gene; (iv) LoxP sites flanking all of the components in (i), (ii) and (iii). FIG. 3B shows the sgRNA lentiviral vector encoding (i) a sgRNA driven by hU6 promoter; (ii) a puromicyn resistant gene and a mKate gene driven by EF1α promoter; (iii) a 2A peptide located between the puromycin resistant gene and mKate gene; (iv) LoxP/lox2272/lox5171 sites flanking the vector components of (ii) and (iii).

First, cells were infected with Cas9_2A_Blast® lentivirus or Cas9_2A_GFP lentivirus. Infected cells were incubated for 48 h before blasticidin S (5 m/mL, Life Technologies) or hygromycin B (250-500 m/mL, Sigma Aldrich) was added to the culture media for selection of cells that were successfully transduced. Selection was kept at least for one week. In a similar fashion, Cas9-expressing cells were transduced with CD47, β2 m or control sgRNA using 100 uL of virus-containing media in the case of mKate-expressing vectors or 25 uL for the rest. Puromycin (5-40 m/mL, Thermo Fisher) was used to select sgRNA-expressing cells. Expression of both Cas9 and sgRNA was confirmed by flow cytometry using GFP and mKate as reporter genes respectively (FIG. 5C). Genome editing was validated by CD47 or β2 m staining at least one week after sgRNA transduction. Cells were stained for surface CD47 expression by flow cytometry. Efficient genome editing (>90%) was achieved after Cas9 and sgRNA delivery with the new vectors. (FIG. 5D). The sgRNA sequences for the control, CD47 and β2 m are as follows:

| | | |
|---|---|---|
| Control: | GCGAGGTATTCGGCTCCGCG | (SEQ ID NO: 3) |
| Cd47: | CCACATTACGGACGATGCAA | (SEQ ID NO: 4) |
| β2m: | AGTATACTCACGCCACCCAC | (SEQ ID NO: 5) |

### Example 3: Transient Expression of Cre Eliminates Vector Components

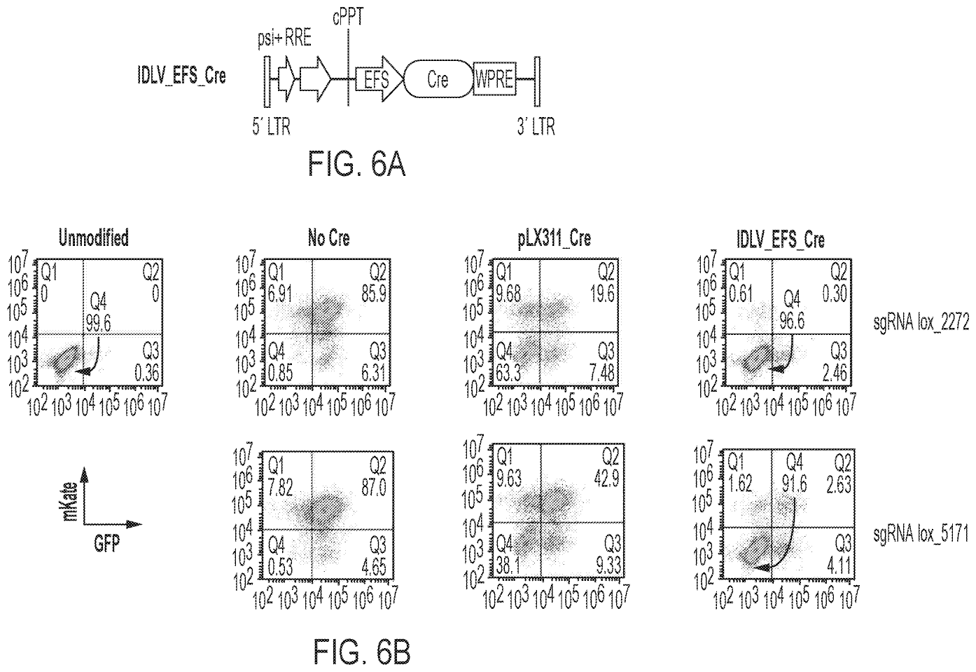

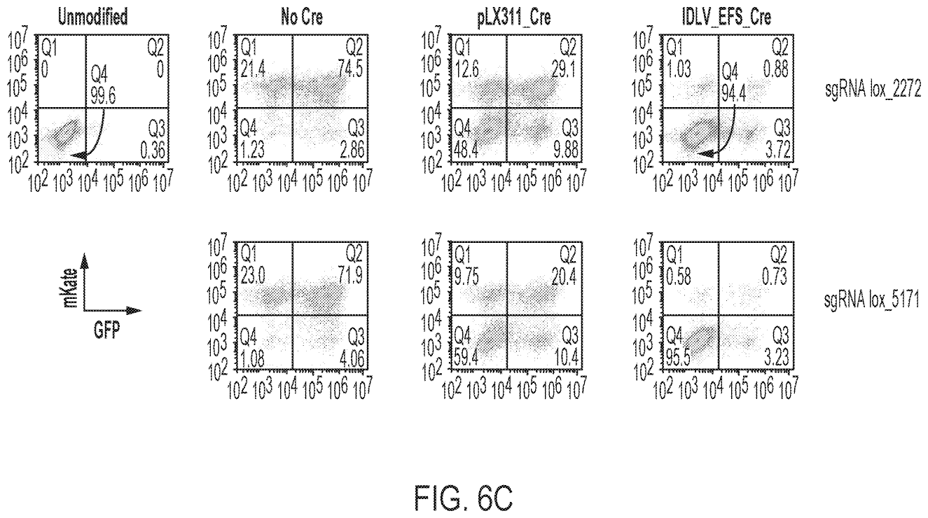

Once the deletion of CD47 or β2 m was successful, Cre was delivered by pLX311_Cre or the Integrase Deficient Lentivirus encoding Cre (IDLV_EFS_Cre) as illustrated by FIG. 6A into the cells. In order to avoid cross-recombination between Cas9 and sgRNA vectors, different lox sequences were used. Cas9 constructs are flanked by LoxP wild type sites whereas sgRNA vectors were designed to include the lox2272 or lox5171 mutated versions. Transient expression of Cre-mediated successful recombination of both Cas9 and sgRNA as observed by loss of fluorescence reporter signal in CT26 cells expressing Cas9_2A_Blast® (FIG. 6B) or Cas9_2A_GFP (FIG. 6C).

### Example 4: Cre-Mediated Recombination and Elimination of Vector Components Restores Normal Tumor Behavior In Vivo

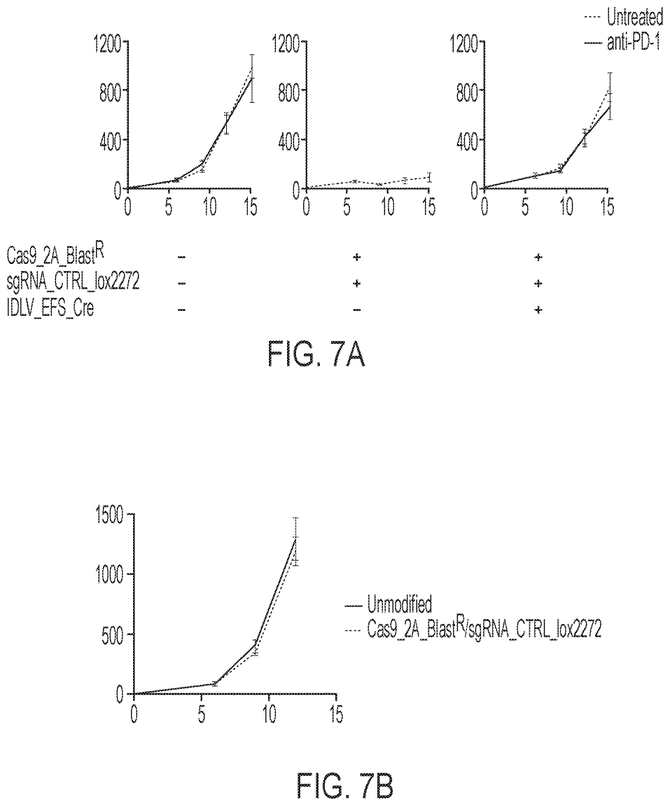

Genetically modified CT26 cells with CRISPR components removed from its genome were used in in vivo tumor experiments to evaluate the immunogenicity of these cells. CT26 cells were inoculated into Balb/c mice. Cas9/sgRNA-expressing tumors (FIG. 7A, middle) were rejected or exhibited an abnormal growth compared to unmodified cells (FIG. 7A, left). Cre-infected cells (FIG. 7A, right) however, showed restored immunogenicity and normal tumor growth in both untreated (dotted lines) and anti-PD-1-treated (solid lines) conditions. Cas9/sgRNA expression did not have any impact in immunodeficient (NSG) mice, suggesting that tumor rejection was caused by the immune system and not due to toxic effects of the vector components (FIG. 7B).

### Example 5: Pooled Genetic Screening for Identification of Cancer Related Genes In Vivo for Cancer Immunotherapy

In silico analysis identified 2368 detectable genes by expression level in CT26 cells as candidates of the in vivo screening. These genes belong to various functional classes. A library of lentiviral vectors, which encode a total of 9,872 sgRNAs targeting these gene candidates was generated. (For additional details, see Manguso R T, et al. “In vivo CRISPR screening identifies Ptpn2 as a cancer immunotherapy target.” Nature (2017) and Lane-Reticker S K, Manguso R T & Haining W N, “Pooled in vivo screens for cancer immunotherapy target discovery.” Immunotherapy (2018), each of which is incorporated herein by reference.) Each sgRNA carried a bar code (a short sequence identifier corresponds to a target gene), which can be used to identify the target gene in a sgRNA transduced cell. CT26 cells were transduced with Cas9 virus (Cas9_2A_Blast) to allow stable expression of Cas9.

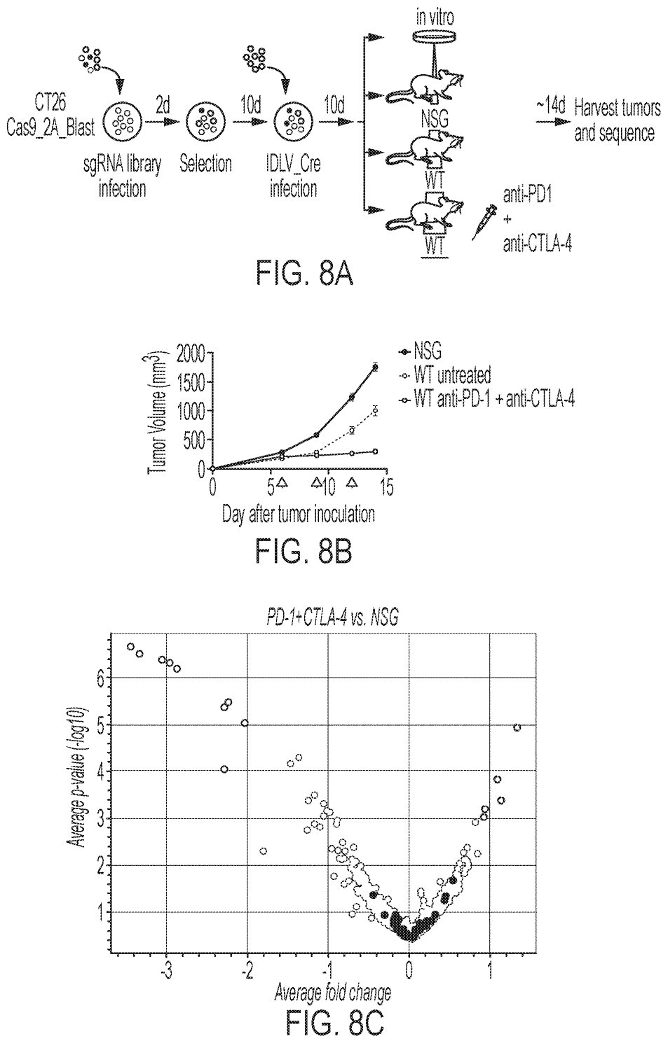

Subsequently, Cas9 expressing CT26 cells were transduced with the pooled sgRNA viruses. Cells were incubated for sufficient time to allow gene editing to take place. The resulting pooled cell population, is a mixture of various genetically modified cells carrying a disrupted gene targeted by the sgRNAs library. The pooled cells were then infected with IDLV_Cre to remove Cas9 and vector components. The sgRNA vectors were designed such that the sgRNA and barcode would remain integrated in the cell genome after Cre treatment. Cells were incubated for sufficient time (about 10 days) for complete genomic excision of Cas9 coding sequence. Since Cre was delivered on an integrase deficient lentiviral vector, its expression was transient and was terminated 10 days post IDLV_Cre infection (FIG. 8A). The resulting CT26 cells were then transplanted onto immune-competent wild type mice by methods described above. Mice were treated with anti-PD-1 and anti-CTLA-4 monoclonal antibodies to generate an adaptive immune response sufficient to apply immune-selective pressure on the transplanted CT26 cells.

In parallel, the pooled genetically modified CT26 cells were transplanted into (NOD-scid IL2RG-null (NSG) immunodeficient mice. Tumor volume was measured at various time points after anti-PD-1 and anti-CTLA-4 monoclonal antibody treatment. The results suggest that the immunotherapy was effective in inhibiting tumor growth in vivo. Moreover, no tumor rejection or exaggerated response to immunotherapy was observed. (FIG. 8B) After 12-14 days, the tumors were harvested from both mouse strains, and genomic DNA from tumor cells was isolated and sequenced for the bar codes. The listing of genes identified by the bar code from tumors in immuno-therapy-treated wild-type mice was compared against the list of genes identified by the bar code from tumours in NSG mice. The results of the screenning were visualized using volcano plots (FIG. 8C). For each gene, the average fold change was calculated as the mean of all four sgRNAs targeting the gene, as shown on the x axis. The x axis shows enrichment (to the left) or depletion (to the right) of the gene. The y axis shows statistical significance as measured by the false discovery rate (FDR)-corrected p value based on STARS analyses. The genes that are highly enriched or highly depleted may be ideal candidates that are related to cancer cell response to immunotherapy.

### EQUIVALENCE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

### LISTING OF VECTOR SEQUENCES

| Cs9 2A Blast: | |
|---|---|
| (SEQ ID NO: 6) | |
| 1 | ACAAGTTTGT ACAAAAAAGT TGGCACCCCC AACTTTATGG ACAAGAAGTA |
| 51 | CAGCATCGGC CTGGACATCG GCACCAACTC TGTGGGCTGG GCCGTGATCA |
| 101 | CCGACGAGTA CAAGGTGCCC AGCAAGAAAT TCAAGGTGCT GGGCAACACC |
| 151 | GACCGGCACA GCATCAAGAA GAACCTGATC GGAGCCCTGC TGTTCGACAG |
| 201 | CGGCGAAACA GCCGAGGCCA CCCGGCTGAA GAGAACCGCC AGAAGAAGAT |
| 251 | ACACCAGACG GAAGAACCGG ATCTGCTATC TGCAAGAGAT CTTCAGCAAC |
| 301 | GAGATGGCCA AGGTGGACGA CAGCTTCTTC CACAGACTGG AAGAGTCCTT |
| 351 | CCTGGTGGAA GAGGATAAGA AGCACGAGCG GCACCCCATC TTCGGCAACA |
| 401 | TCGTGGACGA GGTGGCCTAC CACGAGAAGT ACCCCACCAT CTACCACCTG |
| 451 | AGAAAGAAAC TGGTGGACAG CACCGACAAG GCCGACCTGC GGCTGATCTA |
| 501 | TCTGGCCCTG GCCCACATGA TCAAGTTCCG GGGCCACTTC CTGATCGAGG |
| 551 | GCGACCTGAA CCCCGACAAC AGCGACGTGG ACAAGCTGTT CATCCAGCTG |
| 601 | GTGCAGACCT ACAACCAGCT GTTCGAGGAA AACCCCATCA ACGCCAGCGG |
| 651 | CGTGGACGCC AAGGCCATCC TGTCTGCCAG ACTGAGCAAG AGCAGACGGC |
| 701 | TGGAAAATCT GATCGCCCAG CTGCCCGGCG AGAAGAAGAA TGGCCTGTTC |
| 751 | GGAAACCTGA TTGCCCTGAG CCTGGGCCTG ACCCCCAACT TCAAGAGCAA |
| 801 | CTTCGACCTG GCCGAGGATG CCAAACTGCA GCTGAGCAAG GACACCTACG |
| 851 | ACGACGACCT GGACAACCTG CTGGCCCAGA TCGGCGACCA GTACGCCGAC |
| 901 | CTGTTTCTGG CCGCCAAGAA CCTGTCCGAC GCCATCCTGC TGAGCGACAT |
| 951 | CCTGAGAGTG AACACCGAGA TCACCAAGGC CCCCCTGAGC GCCTCTATGA |
| 1001 | TCAAGAGATA CGACGAGCAC CACCAGGACC TGACCCTGCT GAAAGCTCTC |
| 1051 | GTGCGGCAGC AGCTGCCTGA GAAGTACAAA GAGATTTTCT TCGACCAGAG |
| 1101 | CAAGAACGGC TACGCCGGCT ACATTGACGG CGGAGCCAGC CAGGAAGAGT |
| 1151 | TCTACAAGTT CATCAAGCCC ATCCTGGAAA AGATGGACGG CACCGAGGAA |
| 1201 | CTGCTCGTGA AGCTGAACAG AGAGGACCTG CTGCGGAAGC AGCGGACCTT |
| 1251 | CGACAACGGC AGCATCCCCC ACCAGATCCA CCTGGGAGAG CTGCACGCCA |
| 1301 | TTCTGCGGCG GCAGGAAGAT TTTTACCCAT TCCTGAAGGA CAACCGGGAA |
| 1351 | AAGATCGAGA AGATCCTGAC CTTCCGCATC CCCTACTACG TGGGCCCTCT |
| 1401 | GGCCAGGGGA AACAGCAGAT TCGCCTGGAT GACCAGAAAG AGCGAGGAAA |
| 1451 | CCATCACCCC CTGGAACTTC GAGGAAGTGG TGGACAAGGG CGCTTCCGCC |
| 1501 | CAGAGCTTCA TCGAGCGGAT GACCAACTTC GATAAGAACC TGCCCAACGA |
| 1551 | GAAGGTGCTG CCCAAGCACA GCCTGCTGTA CGAGTACTTC ACCGTGTATA |
| 1601 | ACGAGCTGAC CAAAGTGAAA TACGTGACCG AGGGAATGAG AAAGCCCGCC |
| 1651 | TTCCTGAGCG GCGAGCAGAA AAAGGCCATC GTGGACCTGC TGTTCAAGAC |
| 1701 | CAACCGGAAA GTGACCGTGA AGCAGCTGAA AGAGGACTAC TTCAAGAAAA |
| 1751 | TCGAGTGCTT CGACTCCGTG GAAATCTCCG GCGTGGAAGA TCGGTTCAAC |
| 1801 | GCCTCCCTGG GCACATACCA CGATCTGCTG AAAATTATCA AGGACAAGGA |
| 1851 | CTTCCTGGAC AATGAGGAAA ACGAGGACAT TCTGGAAGAT ATCGTGCTGA |
| 1901 | CCCTGACACT GTTTGAGGAC AGAGAGATGA TCGAGGAACG GCTGAAAACC |
| 1951 | TATGCCCACC TGTTCGACGA CAAAGTGATG AAGCAGCTGA AGCGGCGGAG |
| 2001 | ATACACCGGC TGGGGCAGGC TGAGCCGGAA GCTGATCAAC GGCATCCGGG |
| 2051 | ACAAGCAGTC CGGCAAGACA ATCCTGGATT TCCTGAAGTC CGACGGCTTC |
| 2101 | GCCAACAGAA ACTTCATGCA GCTGATCCAC GACGACAGCC TGACCTTTAA |
| 2151 | AGAGGACATC CAGAAAGCCC AGGTGTCCGG CCAGGGCGAT AGCCTGCACG |
| 2201 | AGCACATTGC CAATCTGGCC GGCAGCCCCG CCATTAAGAA GGGCATCCTG |
| 2251 | CAGACAGTGA AGGTGGTGGA CGAGCTCGTG AAAGTGATGG GCCGGCACAA |
| 2301 | GCCCGAGAAC ATCGTGATCG AAATGGCCAG AGAGAACCAG ACCACCCAGA |
| 2351 | AGGGACAGAA GAACAGCCGC GAGAGAATGA AGCGGATCGA AGAGGGCATC |
| 2401 | AAAGAGCTGG GCAGCCAGAT CCTGAAAGAA CACCCCGTGG AAAACACCCA |
| 2451 | GCTGCAGAAC GAGAAGCTGT ACCTGTACTA CCTGCAGAAT GGGCGGGATA |
| 2501 | TGTACGTGGA CCAGGAACTG GACATCAACC GGCTGTCCGA CTACGATGTG |
| 2551 | GACCATATCG TGCCTCAGAG CTTTCTGAAG GACGACTCCA TCGACAACAA |
| 2601 | GGTGCTGACC AGAAGCGACA AGAACCGGGG CAAGAGCGAC AACGTGCCCT |
| 2651 | CCGAAGAGGT CGTGAAGAAG ATGAAGAACT ACTGGCGGCA GCTGCTGAAC |
| 2701 | GCCAAGCTGA TTACCCAGAG AAAGTTCGAC AATCTGACCA AGGCCGAGAG |
| 2751 | AGGCGGCCTG AGCGAACTGG ATAAGGCCGG CTTCATCAAG AGACAGCTGG |
| 2801 | TGGAAACCCG GCAGATCACA AAGCACGTGG CACAGATCCT GGACTCCCGG |
| 2851 | ATGAACACTA AGTACGACGA GAATGACAAG CTGATCCGGG AAGTGAAAGT |
| 2901 | GATCACCCTG AAGTCCAAGC TGGTGTCCGA TTTCCGGAAG GATTTCCAGT |
| 2951 | TTTACAAAGT GCGCGAGATC AACAACTACC ACCACGCCCA CGACGCCTAC |
| 3001 | CTGAACGCCG TCGTGGGAAC CGCCCTGATC AAAAAGTACC CTAAGCTGGA |
| 3051 | AAGCGAGTTC GTGTACGGCG ACTACAAGGT GTACGACGTG CGGAAGATGA |
| 3101 | TCGCCAAGAG CGAGCAGGAA ATCGGCAAGG CTACCGCCAA GTACTTCTTC |
| 3151 | TACAGCAACA TCATGAACTT TTTCAAGACC GAGATTACCC TGGCCAACGG |
| 3201 | CGAGATCCGG AAGCGGCCTC TGATCGAGAC AAACGGCGAA ACCGGGGAGA |
| 3251 | TCGTGTGGGA TAAGGGCCGG GATTTTGCCA CCGTGCGGAA AGTGCTGAGC |
| 3301 | ATGCCCCAAG TGAATATCGT GAAAAAGACC GAGGTGCAGA CAGGCGGCTT |
| 3351 | CAGCAAAGAG TCTATCCTGC CCAAGAGGAA CAGCGATAAG CTGATCGCCA |
| 3401 | GAAAGAAGGA CTGGGACCCT AAGAAGTACG GCGGCTTCGA CAGCCCCACC |
| 3451 | GTGGCCTATT CTGTGCTGGT GGTGGCCAAA GTGGAAAAGG GCAAGTCCAA |
| 3501 | GAAACTGAAG AGTGTGAAAG AGCTGCTGGG GATCACCATC ATGGAAAGAA |
| 3551 | GCAGCTTCGA GAAGAATCCC ATCGACTTTC TGGAAGCCAA GGGCTACAAA |
| 3601 | GAAGTGAAAA AGGACCTGAT CATCAAGCTG CCTAAGTACT CCCTGTTCGA |
| 3651 | GCTGGAAAAC GGCCGGAAGA GAATGCTGGC CTCTGCCGGC GAACTGCAGA |
| 3701 | AGGGAAACGA ACTGGCCCTG CCCTCCAAAT ATGTGAACTT CCTGTACCTG |
| 3751 | GCCAGCCACT ATGAGAAGCT GAAGGGCTCC CCCGAGGATA ATGAGCAGAA |
| 3801 | ACAGCTGTTT GTGGAACAGC ACAAGCACTA CCTGGACGAG ATCATCGAGC |
| 3851 | AGATCAGCGA GTTCTCCAAG AGAGTGATCC TGGCCGACGC TAATCTGGAC |
| 3901 | AAAGTGCTGT CCGCCTACAA CAAGCACCGG GATAAGCCCA TCAGAGAGCA |
| 3951 | GGCCGAGAAT ATCATCCACC TGTTTACCCT GACCAATCTG GGAGCCCCTG |
| 4001 | CCGCCTTCAA GTACTTTGAC ACCACCATCG ACCGGAAGAG GTACACCAGC |
| 4051 | ACCAAAGAGG TGCTGGACGC CACCCTGATC CACCAGAGCA TCACCGGCCT |
| 4101 | GTACGAGACA CGGATCGACC TGTCTCAGCT GGGAGGCGAC AAGCGACCTG |
| 4151 | CCGCCACAAA GAAGGCTGGA CAGGCTAAGA AGAAGAAAGA TTACAAAGAC |
| 4201 | GATGACGATA AGGGATCCGG CGCAACAAAC TTCTCTCTGC TGAAACAAGC |
| 4251 | CGGAGATGTC GAAGAGAATC CTGGACCGAT GGCCAAGCCT TTGTCTCAAG |
| 4301 | AAGAATCCAC CCTCATTGAA AGAGCAACGG CTACAATCAA CAGCATCCCC |
| 4351 | ATCTCTGAAG ACTACAGCGT CGCCAGCGCA GCTCTCTCTA GCGACGGCCG |
| 4401 | CATCTTCACT GGTGTCAATG TATATCATTT TACTGGGGGA CCTTGTGCAG |
| 4451 | AACTCGTGGT GCTGGGCACT GCTGCTGCTG CGGCAGCTGG CAACCTGACT |
| 4501 | TGTATCGTCG CGATCGGAAA TGAGAACAGG GGCATCTTGA GCCCCTGCGG |
| 4551 | ACGGTGCCGA CAGGTGCTTC TCGATCTGCA TCCTGGGATC AAAGCCATAG |
| 4601 | TGAAGGACAG TGATGGACAG CCGACGGCAG TTGGGATTCG TGAATTGCTG |
| 4651 | CCCTCTGGTT ATGTGTGGGA GGGCTAACTT GTACAAAGTG GTTGATATCG |
| 4701 | GTAAGCCTAT CCCTAACCCT CTCCTCGGTC TCGATTCTAC GTAGTAATGA |
| 4751 | ACTAGTACCG GTTAAGTCGA CAATCAACGC GTTAAGTCGA CAATCAACCT |
| 4801 | CTGGATTACA AAATTTGTGA AAGATTGACT GGTATTCTTA ACTATGTTGC |
| 4851 | TCCTTTTACG CTATGTGGAT ACGCTGCTTT AATGCCTTTG TATCATGCTA |
| 4901 | TTGCTTCCCG TATGGCTTTC ATTTTCTCCT CCTTGTATAA ATCCTGGTTG |
| 4951 | CTGTCTCTTT ATGAGGAGTT GTGGCCCGTT GTCAGGCAAC GTGGCGTGGT |
| 5001 | GTGCACTGTG TTTGCTGACG CAACCCCCAC TGGTTGGGGC ATTGCCACCA |
| 5051 | CCTGTCAGCT CCTTTCCGGG ACTTTCGCTT TCCCCCTCCC TATTGCCACG |
| 5101 | GCGGAACTCA TCGCCGCCTG CCTTGCCCGC TGCTGGACAG GGGCTCGGCT |
| 5151 | GTTGGGCACT GACAATTCCG TGGTGTTGTC GGGGAAATCA TCGTCCTTTC |
| 5201 | CTTGGCTGCT CGCCTGTGTT GCCACCTGGA TTCTGCGCGG GACGTCCTTC |
| 5251 | TGCTACGTCC CTTCGGCCCT CAATCCAGCG GACCTTCCTT CCCGCGGCCT |
| 5301 | GCTGCCGGCT CTGCGGCCTC TTCCGCGTCT TCGCCTTCGC CCTCAGACGA |
| 5351 | GTCGGATCTC CCTTTGGGCC GCCTCCCCGC GTCGACTTTA AGACCAATGA |
| 5401 | CTTACAAGGC AGCTGTAGAT CTTAGCCACT TTTTAAAAGA AAAGGGGGGA |
| 5451 | CTGGAAGGGC TAATTCACTC CCAACGAAGA CAAGATGGGA TCAATTCACC |
| 5501 | ATGGGAATAA CTTCGTATAG CATACATTAT ACGAAGTTAT GCTGCTTTTT |
| 5551 | GCTTGTACTG GGTCTCTCTG GTTAGACCAG ATCTGAGCCT GGGAGCTCTC |
| 5601 | TGGCTAACTA GGGAACCCAC TGCTTAAGCC TCAATAAAGC TTGCCTTGAG |
| 5651 | TGCTTCAAGT AGTGTGTGCC CGTCTGTTGT GTGACTCTGG TAACTAGAGA |
| 5701 | TCCCTCAGAC CCTTTTAGTC AGTGTGGAAA ATCTCTAGCA TACGTATAGT |
| 5751 | AGTTCATGTC ATCTTATTAT TCAGTATTTA TAACTTGCAA AGAAATGAAT |
| 5801 | ATCAGAGAGT GAGAGGAACT TGTTTATTGC AGCTTATAAT GGTTACAAAT |
| 5851 | AAAGCAATAG CATCACAAAT TTCACAAATA AAGCATTTTT TTCACTGCAT |
| 5901 | TCTAGTTGTG GTTTGTCCAA ACTCATCAAT GTATCTTATC ATGTCTGGCT |
| 5951 | CTAGCTATCC CGCCCCTAAC TCCGCCCATC CCGCCCCTAA CTCCGCCCAG |
| 6001 | TTCCGCCCAT TCTCCGCCCC ATGGCTGACT AATTTTTTTT ATTTATGCAG |
| 6051 | AGGCCGAGGC CGCCTCGGCC TCTGAGCTAT TCCAGAAGTA GTGAGGAGGC |
| 6101 | TTTTTTGGAG GCCTAGGGAC GTACCCAATT CGCCCTATAG TGAGTCGTAT |
| 6151 | TACGCGCGCT CACTGGCCGT CGTTTTACAA CGTCGTGACT GGGAAAACCC |
| 6201 | TGGCGTTACC CAACTTAATC GCCTTGCAGC ACATCCCCCT TTCGCCAGCT |
| 6251 | GGCGTAATAG CGAAGAGGCC CGCACCGATC GCCCTTCCCA ACAGTTGCGC |
| 6301 | AGCCTGAATG GCGAATGGGA CGCGCCCTGT AGCGGCGCAT TAAGCGCGGC |
| 6351 | GGGTGTGGTG GTTACGCGCA GCGTGACCGC TACACTTGCC AGCGCCCTAG |
| 6401 | CGCCCGCTCC TTTCGCTTTC TTCCCTTCCT TTCTCGCCAC GTTCGCCGGC |
| 6451 | TTTCCCCGTC AAGCTCTAAA TCGGGGGCTC CCTTTAGGGT TCCGATTTAG |
| 6501 | TGCTTTACGG CACCTCGACC CCAAAAAACT TGATTAGGGT GATGGTTCAC |
| 6551 | GTAGTGGGCC ATCGCCCTGA TAGACGGTTT TTCGCCCTTT GACGTTGGAG |
| 6601 | TCCACGTTCT TTAATAGTGG ACTCTTGTTC CAAACTGGAA CAACACTCAA |
| 6651 | CCCTATCTCG GTCTATTCTT TTGATTTATA AGGGATTTTG CCGATTTCGG |
| 6701 | CCTATTGGTT AAAAAATGAG CTGATTTAAC AAAAATTTAA CGCGAATTTT |
| 6751 | AACAAAATAT TAACGCTTAC AATTTAGGTG GCACTTTTCG GGGAAATGTG |
| 6801 | CGCGGAACCC CTATTTGTTT ATTTTTCTAA ATACATTCAA ATATGTATCC |
| 6851 | GCTCATGAGA CAATAACCCT GATAAATGCT TCAATAATAT TGAAAAAGGA |
| 6901 | AGAGTATGAG TATTCAACAT TTCCGTGTCG CCCTTATTCC CTTTTTTGCG |
| 6951 | GCATTTTGCC TTCCTGTTTT TGCTCACCCA GAAACGCTGG TGAAAGTAAA |
| 7001 | AGATGCTGAA GATCAGTTGG GTGCACGAGT GGGTTACATC GAACTGGATC |
| 7051 | TCAACAGCGG TAAGATCCTT GAGAGTTTTC GCCCCGAAGA ACGTTTTCCA |
| 7101 | ATGATGAGCA CTTTTAAAGT TCTGCTATGT GGCGCGGTAT TATCCCGTAT |
| 7151 | TGACGCCGGG CAAGAGCAAC TCGGTCGCCG CATACACTAT TCTCAGAATG |
| 7201 | ACTTGGTTGA GTACTCACCA GTCACAGAAA AGCATCTTAC GGATGGCATG |
| 7251 | ACAGTAAGAG AATTATGCAG TGCTGCCATA ACCATGAGTG ATAACACTGC |
| 7301 | GGCCAACTTA CTTCTGACAA CGATCGGAGG ACCGAAGGAG CTAACCGCTT |
| 7351 | TTTTGCACAA CATGGGGGAT CATGTAACTC GCCTTGATCG TTGGGAACCG |
| 7401 | GAGCTGAATG AAGCCATACC AAACGACGAG CGTGACACCA CGATGCCTGT |
| 7451 | AGCAATGGCA ACAACGTTGC GCAAACTATT AACTGGCGAA CTACTTACTC |
| 7501 | TAGCTTCCCG GCAACAATTA ATAGACTGGA TGGAGGCGGA TAAAGTTGCA |
| 7551 | GGACCACTTC TGCGCTCGGC CCTTCCGGCT GGCTGGTTTA TTGCTGATAA |
| 7601 | ATCTGGAGCC GGTGAGCGTG GGTCTCGCGG TATCATTGCA GCACTGGGGC |
| 7651 | CAGATGGTAA GCCCTCCCGT ATCGTAGTTA TCTACACGAC GGGGAGTCAG |
| 7701 | GCAACTATGG ATGAACGAAA TAGACAGATC GCTGAGATAG GTGCCTCACT |
| 7751 | GATTAAGCAT TGGTAACTGT CAGACCAAGT TTACTCATAT ATACTTTAGA |
| 7801 | TTGATTTAAA ACTTCATTTT TAATTTAAAA GGATCTAGGT GAAGATCCTT |
| 7851 | TTTGATAATC TCATGACCAA AATCCCTTAA CGTGAGTTTT CGTTCCACTG |
| 7901 | AGCGTCAGAC CCCGTAGAAA AGATCAAAGG ATCTTCTTGA GATCCTTTTT |
| 7951 | TTCTGCGCGT AATCTGCTGC TTGCAAACAA AAAAACCACC GCTACCAGCG |
| 8001 | GTGGTTTGTT TGCCGGATCA AGAGCTACCA ACTCTTTTTC CGAAGGTAAC |
| 8051 | TGGCTTCAGC AGAGCGCAGA TACCAAATAC TGTTCTTCTA GTGTAGCCGT |
| 8101 | AGTTAGGCCA CCACTTCAAG AACTCTGTAG CACCGCCTAC ATACCTCGCT |
| 8151 | CTGCTAATCC TGTTACCAGT GGCTGCTGCC AGTGGCGATA AGTCGTGTCT |
| 8201 | TACCGGGTTG GACTCAAGAC GATAGTTACC GGATAAGGCG CAGCGGTCGG |
| 8251 | GCTGAACGGG GGGTTCGTGC ACACAGCCCA GCTTGGAGCG AACGACCTAC |
| 8301 | ACCGAACTGA GATACCTACA GCGTGAGCTA TGAGAAAGCG CCACGCTTCC |
| 8351 | CGAAGGGAGA AAGGCGGACA GGTATCCGGT AAGCGGCAGG GTCGGAACAG |
| 8401 | GAGAGCGCAC GAGGGAGCTT CCAGGGGGAA ACGCCTGGTA TCTTTATAGT |
| 8451 | CCTGTCGGGT TTCGCCACCT CTGACTTGAG CGTCGATTTT TGTGATGCTC |
| 8501 | GTCAGGGGGG CGGAGCCTAT GGAAAAACGC CAGCAACGCG GCCTTTTTAC |
| 8551 | GGTTCCTGGC CTTTTGCTGG CCTTTTGCTC ACATGTTCTT TCCTGCGTTA |
| 8601 | TCCCCTGATT CTGTGGATAA CCGTATTACC GCCTTTGAGT GAGCTGATAC |
| 8651 | CGCTCGCCGC AGCCGAACGA CCGAGCGCAG CGAGTCAGTG AGCGAGGAAG |
| 8701 | CGGAAGAGCG CCCAATACGC AAACCGCCTC TCCCCGCGCG TTGGCCGATT |
| 8751 | CATTAATGCA GCTGGCACGA CAGGTTTCCC GACTGGAAAG CGGGCAGTGA |
| 8801 | GCGCAACGCA ATTAATGTGA GTTAGCTCAC TCATTAGGCA CCCCAGGCTT |
| 8851 | TACACTTTAT GCTTCCGGCT CGTATGTTGT GTGGAATTGT GAGCGGATAA |
| 8901 | CAATTTCACA CAGGAAACAG CTATGACCAT GATTACGCCA AGCGCGCAAT |
| 8951 | TAACCCTCAC TAAAGGGAAC AAAAGCTGGA GCTGCAAGCT TAATGTAGTC |
| 9001 | TTATGCAATA CTCTTGTAGT CTTGCAACAT GGTAACGATG AGTTAGCAAC |
| 9051 | ATGCCTTACA AGGAGAGAAA AAGCACCGTG CATGCCGATT GGTGGAAGTA |
| 9101 | AGGTGGTACG ATCGTGCCTT ATTAGGAAGG CAACAGACGG GTCTGACATG |
| 9151 | GATTGGACGA ACCACTGAAT TGCCGCATTG CAGAGATATT GTATTTAAGT |
| 9201 | GCCTAGCTCG ATACATAAAC GGGTCTCTCT GGTTAGACCA GATCTGAGCC |
| 9251 | TGGGAGCTCT CTGGCTAACT AGGGAACCCA CTGCTTAAGC CTCAATAAAG |
| 9301 | CTTGCCTTGA GTGCTTCAAG TAGTGTGTGC CCGTCTGTTG TGTGACTCTG |
| 9351 | GTAACTAGAG ATCCCTCAGA CCCTTTTAGT CAGTGTGGAA AATCTCTAGC |
| 9401 | AGTGGCGCCC GAACAGGGAC TTGAAAGCGA AAGGGAAACC AGAGGAGCTC |
| 9451 | TCTCGACGCA GGACTCGGCT TGCTGAAGCG CGCACGGCAA GAGGCGAGGG |
| 9501 | GCGGCGACTG GTGAGTACGC CAAAAATTTT GACTAGCGGA GGCTAGAAGG |
| 9551 | AGAGAGATGG GTGCGAGAGC GTCAGTATTA AGCGGGGGAG AATTAGATCG |
| 9601 | CGATGGGAAA AAATTCGGTT AAGGCCAGGG GGAAAGAAAA AATATAAATT |
| 9651 | AAAACATATA GTATGGGCAA GCAGGGAGCT AGAACGATTC GCAGTTAATC |
| 9701 | CTGGCCTGTT AGAAACATCA GAAGGCTGTA GACAAATACT GGGACAGCTA |
| 9751 | CAACCATCCC TTCAGACAGG ATCAGAAGAA CTTAGATCAT TATATAATAC |
| 9801 | AGTAGCAACC CTCTATTGTG TGCATCAAAG GATAGAGATA AAAGACACCA |
| 9851 | AGGAAGCTTT AGACAAGATA GAGGAAGAGC AAAACAAAAG TAAGACCACC |
| 9901 | GCACAGCAAG CGGCCGCTGA TCTTCAGACC TGGAGGAGGA GATATGAGGG |
| 9951 | ACAATTGGAG AAGTGAATTA TATAAATATA AAGTAGTAAA AATTGAACCA |
| 10001 | TTAGGAGTAG CACCCACCAA GGCAAAGAGA AGAGTGGTGC AGAGAGAAAA |
| 10051 | AAGAGCAGTG GGAATAGGAG CTTTGTTCCT TGGGTTCTTG GGAGCAGCAG |
| 10101 | GAAGCACTAT GGGCGCAGCG TCAATGACGC TGACGGTACA GGCCAGACAA |
| 10151 | TTATTGTCTG GTATAGTGCA GCAGCAGAAC AATTTGCTGA GGGCTATTGA |
| 10201 | GGCGCAACAG CATCTGTTGC AACTCACAGT CTGGGGCATC AAGCAGCTCC |
| 10251 | AGGCAAGAAT CCTGGCTGTG GAAAGATACC TAAAGGATCA ACAGCTCCTG |
| 10301 | GGGATTTGGG GTTGCTCTGG AAAACTCATT TGCACCACTG CTGTGCCTTG |
| 10351 | GAATGCTAGT TGGAGTAATA AATCTCTGGA ACAGATTTGG AATCACACGA |
| 10401 | CCTGGATGGA GTGGGACAGA GAAATTAACA ATTACACAAG CTTAATACAC |
| 10451 | TCCTTAATTG AAGAATCGCA AAACCAGCAA GAAAAGAATG AACAAGAATT |
| 10501 | ATTGGAATTA GATAAATGGG CAAGTTTGTG GAATTGGTTT AACATAACAA |
| 10551 | ATTGGCTGTG GTATATAAAA TTATTCATAA TGATAGTAGG AGGCTTGGTA |
| 10601 | GGTTTAAGAA TAGTTTTTGC TGTACTTTCT ATAGTGAATA GAGTTAGGCA |
| 10651 | GGGATATTCA CCATTATCGT TTCAGACCCA CCTCCCAACC CCGAGGGGAC |
| 10701 | CCATGCATTG CATCTCAATT AGTCAGCAAC CAGGTGTGGA AAGTCCCCAG |
| 10751 | GCTCCCCAGC AGGCAGAAGT ATGCAAAGCA TGCGTCTCAA TTAGTCAGCA |
| 10801 | ACCATAGTCC CGCCCCTAAC TCCGCCCATC CCGCCCCTAA CTCCGCCCAG |
| 10851 | TTCCGCCCAT TCTCCGCCCC ATGGCTGACT AATTTTTTTT ATTTATGCAG |
| 10901 | AGGCCGAGGC CGCCTCGGCC TCTGAGCTAT TCCAGAAGTA GTGAGGAGGC |
| 10951 | TTTTTTGGAG GCCTAGGCTT TTGCAAAAAG CTTTCTAGAG GTACCACCAT |
| 11001 | GGTGAGCAAG GGCGAGGAGC TGTTCACCGG GGTGGTGCCC ATCCTGGTCG |
| 11051 | AGCTGGACGG CGACGTAAAC GGCCACAAGT TCAGCGTGTC TGGCGAGGGC |
| 11101 | GAGGGCGATG CCACCTACGG CAAGCTGACC CTGAAGTTCA TCTGCACCAC |
| 11151 | CGGCAAGCTG CCCGTGCCCT GGCCCACCCT CGTGACCACC CTGACCTACG |
| 11201 | GCGTGCAGTG CTTCAGCCGC TACCCCGACC ACATGAAGCA GCACGACTTC |
| 11251 | TTCAAGTCCG CCATGCCCGA AGGCTACGTC CAGGAGCGCA CCATCTTCTT |
| 11301 | CAAGGACGAC GGCAACTACA AGACCCGCGC CGAGGTGAAG TTCGAGGGCG |
| 11351 | ACACCCTGGT GAACCGCATC GAGCTGAAGG GCATCGACTT CAAGGAGGAC |
| 11401 | GGCAACATCC TGGGGCACAA GCTGGAGTAC AACTACAACA GCCACAACGT |
| 11451 | CTATATCATG GCCGACAAGC AGAAGAACGG CATCAAGGTG AACTTCAAGA |
| 11501 | TCCGCCACAA CATCGAGGAC GGCAGCGTGC AGCTCGCCGA CCACTACCAG |
| 11551 | CAGAACACCC CCATCGGCGA CGGCCCCGTG CTGCTGCCCG ACAACCACTA |
| 11601 | CCTGAGCACC CAGTCCGCCC TGAGCAAAGA CCCCAACGAG AAGCGCGATC |
| 11651 | ACATGGTCCT GCTGGAGTTC GTGACCGCCG CCGGGATCAC TCTCGGCATG |
| 11701 | GACGAGCTGT ACAAGTCCTA AGGCGCGCCG TTAACGAATT CTAGATCTTG |
| 11751 | AGACAAATGG CAGTATTCAT CCACAATTTT AAAAGAAAAG GGGGGATTGG |
| 11801 | GGGGTACAGT GCAGGGGAAA GAATAGTAGA CATAATAGCA ACAGACATAC |
| 11851 | AAACTAAAGA ATTACAAAAA CAAATTACAA AAATTCAAAA TTTTCGGGTT |
| 11901 | TATTACAGGG ACAGCAGAGA TCCACTTTGG CGCCGGCTCG AGGCCTGCAG |
| 11951 | GTGCAAAGAT GGATAAAGTT TTAAACAGAG AGGAATCTTT GCAGCTAATG |
| 12001 | GACCTTCTAG GTCTTGAAAG GAGTGGGAAT TGGCTCCGGT GCCCGTCAGT |
| 12051 | GGGCAGAGCG CACATCGCCC ACAGTCCCCG AGAAGTTGGG GGGAGGGGTC |
| 12101 | GGCAATTGAA CCGGTGCCTA GAGAAGGTGG CGCGGGGTAA ACTGGGAAAG |
| 12151 | TGATGTCGTG TACTGGCTCC GCCTTTTTCC CGAGGGTGGG GGAGAACCGT |
| 12201 | ATATAAGTGC AGTAGTCGCC GTGAACGTTC TTTTTCGCAA CGGGTTTGCC |
| 12251 | GCCAGAACAC AGGTAAGTGC CGTGTGTGGT TCCCGCGGGC CTGGCCTCTT |
| 12301 | TACGGGTTAT GGCCCTTGCG TGCCTTGAAT TACTTCCACC TGGCTGCAGT |
| 12351 | ACGTGATTCT TGATCCCGAG CTTCGGGTTG GAAGTGGGTG GGAGAGTTCG |
| 12401 | AGGCCTTGCG CTTAAGGAGC CCCTTCGCCT CGTGCTTGAG TTGAGGCCTG |
| 12451 | GCCTGGGCGC TGGGGCCGCC GCGTGCGAAT CTGGTGGCAC CTTCGCGCCT |
| 12501 | GTCTCGCTGC TTTCGATAAG TCTCTAGCCA TTTAAAATTT TTGATGACCT |
| 12551 | GCTGCGACGC TTTTTTTCTG GCAAGATAGT CTTGTAAATG CGGGCCAAGA |
| 12601 | TCTGCACACT GGTATTTCGG TTTTTGGGGC CGCGGGCGGC GACGGGGCCC |
| 12651 | GTGCGTCCCA GCGCACATGT TCGGCGAGGC GGGGCCTGCG AGCGCGGCCA |
| 12701 | CCGAGAATCG GACGGGGGTA GTCTCAAGCT GGCCGGCCTG CTCTGGTGCC |
| 12751 | TGGCCTCGCG CCGCCGTGTA TCGCCCCGCC CTGGGCGGCA AGGCTGGCCC |
| 12801 | GGTCGGCACC AGTTGCGTGA GCGGAAAGAT GGCCGCTTCC CGGCCCTGCT |
| 12851 | GCAGGGAGCT CAAAATGGAG GACGCGGCGC TCGGGAGAGC GGGCGGGTGA |
| 12901 | GTCACCCACA CAAAGGAAAA GGGCCTTTCC GTCCTCAGCC GTCGCTTCAT |
| 12951 | GTGACTCCAC GGAGTACCGG GCGCCGTCCA GGCACCTCGA TTAGTTCTCG |
| 13001 | AGCTTTTGGA GTACGTCGTC TTTAGGTTGG GGGGAGGGGT TTTATGCGAT |
| 13051 | GGAGTTTCCC CACACTGAGT GGGTGGAGAC TGAAGTTAGG CCAGCTTGGC |
| 13101 | ACTTGATGTA ATTCTCCTTG GAATTTGCCC TTTTTGAGTT TGGATCTTGG |
| 13151 | TTCATTCTCA AGCCTCAGAC AGTGGTTCAA AGTTTTTTTC TTCCATTTCA |
| 13201 | GGTGTCGTGA GGCTAGCATC GATTGATCA |

### ANNOTATIONS

1-5: attR1
37-4140: S. Pyogenes Cas9
4141-4188: NLS (nucleoplasmin): Nuclear localization sequence of nucleoplasmin
4189-4212: FLAG
4213-4278: P2A
4279-4674: BlastR
4678-4692: attR2
4700-4741: V5 tag
4792-5380: WPRE
5435-5450: cPPT
5507-5540: loxP: one lox P site
5560-5740: HIV-1 3′ LTR
5817-5947: SV40 polyadenylation signal
6027-6102: SV40 origin of replication
6320-6775: F1 ori
6906-7766: AmpR
7914-8581: pUC ori
8990-9402: 5′ LTR
9453-9590: psi
9557-9921: gag
10067-10308: Rev response element (RRE)
10709-10983: SV40 (promoter)
10996-11721: EGFP
11777-11894: cPPT
11952-13211: EF1α (promoter)

| Cas9 2A GFP: | |
|---|---|
| (SEQ ID NO: 7) | |
| 1 | CTCGAGGCCT GCAGGTGCAA AGATGGATAA AGTTTTAAAC AGAGAGGAAT |
| 51 | CTTTGCAGCT AATGGACCTT CTAGGTCTTG AAAGGAGTGG GAATTGGCTC |
| 101 | CGGTGCCCGT CAGTGGGCAG AGCGCACATC GCCCACAGTC CCCGAGAAGT |
| 151 | TGGGGGGAGG GGTCGGCAAT TGAACCGGTG CCTAGAGAAG GTGGCGCGGG |
| 201 | GTAAACTGGG AAAGTGATGT CGTGTACTGG CTCCGCCTTT TTCCCGAGGG |
| 251 | TGGGGGAGAA CCGTATATAA GTGCAGTAGT CGCCGTGAAC GTTCTTTTTC |
| 301 | GCAACGGGTT TGCCGCCAGA ACACAGGTAA GTGCCGTGTG TGGTTCCCGC |
| 351 | GGGCCTGGCC TCTTTACGGG TTATGGCCCT TGCGTGCCTT GAATTACTTC |
| 401 | CACCTGGCTG CAGTACGTGA TTCTTGATCC CGAGCTTCGG GTTGGAAGTG |
| 451 | GGTGGGAGAG TTCGAGGCCT TGCGCTTAAG GAGCCCCTTC GCCTCGTGCT |
| 501 | TGAGTTGAGG CCTGGCCTGG GCGCTGGGGC CGCCGCGTGC GAATCTGGTG |
| 551 | GCACCTTCGC GCCTGTCTCG CTGCTTTCGA TAAGTCTCTA GCCATTTAAA |
| 601 | ATTTTTGATG ACCTGCTGCG ACGCTTTTTT TCTGGCAAGA TAGTCTTGTA |
| 651 | AATGCGGGCC AAGATCTGCA CACTGGTATT TCGGTTTTTG GGGCCGCGGG |
| 701 | CGGCGACGGG GCCCGTGCGT CCCAGCGCAC ATGTTCGGCG AGGCGGGGCC |
| 751 | TGCGAGCGCG GCCACCGAGA ATCGGACGGG GGTAGTCTCA AGCTGGCCGG |
| 801 | CCTGCTCTGG TGCCTGGCCT CGCGCCGCCG TGTATCGCCC CGCCCTGGGC |
| 851 | GGCAAGGCTG GCCCGGTCGG CACCAGTTGC GTGAGCGGAA AGATGGCCGC |
| 901 | TTCCCGGCCC TGCTGCAGGG AGCTCAAAAT GGAGGACGCG GCGCTCGGGA |
| 951 | GAGCGGGCGG GTGAGTCACC CACACAAAGG AAAAGGGCCT TTCCGTCCTC |
| 1001 | AGCCGTCGCT TCATGTGACT CCACGGAGTA CCGGGCGCCG TCCAGGCACC |
| 1051 | TCGATTAGTT CTCGAGCTTT TGGAGTACGT CGTCTTTAGG TTGGGGGGAG |
| 1101 | GGGTTTTATG CGATGGAGTT TCCCCACACT GAGTGGGTGG AGACTGAAGT |
| 1151 | TAGGCCAGCT TGGCACTTGA TGTAATTCTC CTTGGAATTT GCCCTTTTTG |
| 1201 | AGTTTGGATC TTGGTTCATT CTCAAGCCTC AGACAGTGGT TCAAAGTTTT |
| 1251 | TTTCTTCCAT TTCAGGTGTC GTGAGGCTAG CATCGATTGA TCAACAAGTT |
| 1301 | TGTACAAAAA AGTTGGCACC CCCAACTTTA TGGACAAGAA GTACAGCATC |
| 1351 | GGCCTGGACA TCGGCACCAA CTCTGTGGGC TGGGCCGTGA TCACCGACGA |
| 1401 | GTACAAGGTG CCCAGCAAGA AATTCAAGGT GCTGGGCAAC ACCGACCGGC |
| 1451 | ACAGCATCAA GAAGAACCTG ATCGGAGCCC TGCTGTTCGA CAGCGGCGAA |
| 1501 | ACAGCCGAGG CCACCCGGCT GAAGAGAACC GCCAGAAGAA GATACACCAG |
| 1551 | ACGGAAGAAC CGGATCTGCT ATCTGCAAGA GATCTTCAGC AACGAGATGG |
| 1601 | CCAAGGTGGA CGACAGCTTC TTCCACAGAC TGGAAGAGTC CTTCCTGGTG |
| 1651 | GAAGAGGATA AGAAGCACGA GCGGCACCCC ATCTTCGGCA ACATCGTGGA |
| 1701 | CGAGGTGGCC TACCACGAGA AGTACCCCAC CATCTACCAC CTGAGAAAGA |
| 1751 | AACTGGTGGA CAGCACCGAC AAGGCCGACC TGCGGCTGAT CTATCTGGCC |
| 1801 | CTGGCCCACA TGATCAAGTT CCGGGGCCAC TTCCTGATCG AGGGCGACCT |
| 1851 | GAACCCCGAC AACAGCGACG TGGACAAGCT GTTCATCCAG CTGGTGCAGA |
| 1901 | CCTACAACCA GCTGTTCGAG GAAAACCCCA TCAACGCCAG CGGCGTGGAC |
| 1951 | GCCAAGGCCA TCCTGTCTGC CAGACTGAGC AAGAGCAGAC GGCTGGAAAA |
| 2001 | TCTGATCGCC CAGCTGCCCG GCGAGAAGAA GAATGGCCTG TTCGGAAACC |
| 2051 | TGATTGCCCT GAGCCTGGGC CTGACCCCCA ACTTCAAGAG CAACTTCGAC |
| 2101 | CTGGCCGAGG ATGCCAAACT GCAGCTGAGC AAGGACACCT ACGACGACGA |
| 2151 | CCTGGACAAC CTGCTGGCCC AGATCGGCGA CCAGTACGCC GACCTGTTTC |
| 2201 | TGGCCGCCAA GAACCTGTCC GACGCCATCC TGCTGAGCGA CATCCTGAGA |
| 2251 | GTGAACACCG AGATCACCAA GGCCCCCCTG AGCGCCTCTA TGATCAAGAG |
| 2301 | ATACGACGAG CACCACCAGG ACCTGACCCT GCTGAAAGCT CTCGTGCGGC |
| 2351 | AGCAGCTGCC TGAGAAGTAC AAAGAGATTT TCTTCGACCA GAGCAAGAAC |
| 2401 | GGCTACGCCG GCTACATTGA CGGCGGAGCC AGCCAGGAAG AGTTCTACAA |
| 2451 | GTTCATCAAG CCCATCCTGG AAAAGATGGA CGGCACCGAG GAACTGCTCG |
| 2501 | TGAAGCTGAA CAGAGAGGAC CTGCTGCGGA AGCAGCGGAC CTTCGACAAC |
| 2551 | GGCAGCATCC CCCACCAGAT CCACCTGGGA GAGCTGCACG CCATTCTGCG |
| 2601 | GCGGCAGGAA GATTTTTACC CATTCCTGAA GGACAACCGG GAAAAGATCG |
| 2651 | AGAAGATCCT GACCTTCCGC ATCCCCTACT ACGTGGGCCC TCTGGCCAGG |
| 2701 | GGAAACAGCA GATTCGCCTG GATGACCAGA AAGAGCGAGG AAACCATCAC |
| 2751 | CCCCTGGAAC TTCGAGGAAG TGGTGGACAA GGGCGCTTCC GCCCAGAGCT |
| 2801 | TCATCGAGCG GATGACCAAC TTCGATAAGA ACCTGCCCAA CGAGAAGGTG |
| 2851 | CTGCCCAAGC ACAGCCTGCT GTACGAGTAC TTCACCGTGT ATAACGAGCT |
| 2901 | GACCAAAGTG AAATACGTGA CCGAGGGAAT GAGAAAGCCC GCCTTCCTGA |
| 2951 | GCGGCGAGCA GAAAAAGGCC ATCGTGGACC TGCTGTTCAA GACCAACCGG |
| 3001 | AAAGTGACCG TGAAGCAGCT GAAAGAGGAC TACTTCAAGA AAATCGAGTG |
| 3051 | CTTCGACTCC GTGGAAATCT CCGGCGTGGA AGATCGGTTC AACGCCTCCC |
| 3101 | TGGGCACATA CCACGATCTG CTGAAAATTA TCAAGGACAA GGACTTCCTG |
| 3151 | GACAATGAGG AAAACGAGGA CATTCTGGAA GATATCGTGC TGACCCTGAC |
| 3201 | ACTGTTTGAG GACAGAGAGA TGATCGAGGA ACGGCTGAAA ACCTATGCCC |
| 3251 | ACCTGTTCGA CGACAAAGTG ATGAAGCAGC TGAAGCGGCG GAGATACACC |
| 3301 | GGCTGGGGCA GGCTGAGCCG GAAGCTGATC AACGGCATCC GGGACAAGCA |
| 3351 | GTCCGGCAAG ACAATCCTGG ATTTCCTGAA GTCCGACGGC TTCGCCAACA |
| 3401 | GAAACTTCAT GCAGCTGATC CACGACGACA GCCTGACCTT TAAAGAGGAC |
| 3451 | ATCCAGAAAG CCCAGGTGTC CGGCCAGGGC GATAGCCTGC ACGAGCACAT |
| 3501 | TGCCAATCTG GCCGGCAGCC CCGCCATTAA GAAGGGCATC CTGCAGACAG |
| 3551 | TGAAGGTGGT GGACGAGCTC GTGAAAGTGA TGGGCCGGCA CAAGCCCGAG |
| 3601 | AACATCGTGA TCGAAATGGC CAGAGAGAAC CAGACCACCC AGAAGGGACA |
| 3651 | GAAGAACAGC CGCGAGAGAA TGAAGCGGAT CGAAGAGGGC ATCAAAGAGC |
| 3701 | TGGGCAGCCA GATCCTGAAA GAACACCCCG TGGAAAACAC CCAGCTGCAG |
| 3751 | AACGAGAAGC TGTACCTGTA CTACCTGCAG AATGGGCGGG ATATGTACGT |
| 3801 | GGACCAGGAA CTGGACATCA ACCGGCTGTC CGACTACGAT GTGGACCATA |
| 3851 | TCGTGCCTCA GAGCTTTCTG AAGGACGACT CCATCGACAA CAAGGTGCTG |
| 3901 | ACCAGAAGCG ACAAGAACCG GGGCAAGAGC GACAACGTGC CCTCCGAAGA |
| 3951 | GGTCGTGAAG AAGATGAAGA ACTACTGGCG GCAGCTGCTG AACGCCAAGC |
| 4001 | TGATTACCCA GAGAAAGTTC GACAATCTGA CCAAGGCCGA GAGAGGCGGC |
| 4051 | CTGAGCGAAC TGGATAAGGC CGGCTTCATC AAGAGACAGC TGGTGGAAAC |
| 4101 | CCGGCAGATC ACAAAGCACG TGGCACAGAT CCTGGACTCC CGGATGAACA |
| 4151 | CTAAGTACGA CGAGAATGAC AAGCTGATCC GGGAAGTGAA AGTGATCACC |
| 4201 | CTGAAGTCCA AGCTGGTGTC CGATTTCCGG AAGGATTTCC AGTTTTACAA |
| 4251 | AGTGCGCGAG ATCAACAACT ACCACCACGC CCACGACGCC TACCTGAACG |
| 4301 | CCGTCGTGGG AACCGCCCTG ATCAAAAAGT ACCCTAAGCT GGAAAGCGAG |
| 4351 | TTCGTGTACG GCGACTACAA GGTGTACGAC GTGCGGAAGA TGATCGCCAA |
| 4401 | GAGCGAGCAG GAAATCGGCA AGGCTACCGC CAAGTACTTC TTCTACAGCA |
| 4451 | ACATCATGAA CTTTTTCAAG ACCGAGATTA CCCTGGCCAA CGGCGAGATC |
| 4501 | CGGAAGCGGC CTCTGATCGA GACAAACGGC GAAACCGGGG AGATCGTGTG |
| 4551 | GGATAAGGGC CGGGATTTTG CCACCGTGCG GAAAGTGCTG AGCATGCCCC |
| 4601 | AAGTGAATAT CGTGAAAAAG ACCGAGGTGC AGACAGGCGG CTTCAGCAAA |
| 4651 | GAGTCTATCC TGCCCAAGAG GAACAGCGAT AAGCTGATCG CCAGAAAGAA |
| 4701 | GGACTGGGAC CCTAAGAAGT ACGGCGGCTT CGACAGCCCC ACCGTGGCCT |
| 4751 | ATTCTGTGCT GGTGGTGGCC AAAGTGGAAA AGGGCAAGTC CAAGAAACTG |
| 4801 | AAGAGTGTGA AAGAGCTGCT GGGGATCACC ATCATGGAAA GAAGCAGCTT |
| 4851 | CGAGAAGAAT CCCATCGACT TTCTGGAAGC CAAGGGCTAC AAAGAAGTGA |
| 4901 | AAAAGGACCT GATCATCAAG CTGCCTAAGT ACTCCCTGTT CGAGCTGGAA |
| 4951 | AACGGCCGGA AGAGAATGCT GGCCTCTGCC GGCGAACTGC AGAAGGGAAA |
| 5001 | CGAACTGGCC CTGCCCTCCA AATATGTGAA CTTCCTGTAC CTGGCCAGCC |
| 5051 | ACTATGAGAA GCTGAAGGGC TCCCCCGAGG ATAATGAGCA GAAACAGCTG |
| 5101 | TTTGTGGAAC AGCACAAGCA CTACCTGGAC GAGATCATCG AGCAGATCAG |
| 5151 | CGAGTTCTCC AAGAGAGTGA TCCTGGCCGA CGCTAATCTG GACAAAGTGC |
| 5201 | TGTCCGCCTA CAACAAGCAC CGGGATAAGC CCATCAGAGA GCAGGCCGAG |
| 5251 | AATATCATCC ACCTGTTTAC CCTGACCAAT CTGGGAGCCC CTGCCGCCTT |
| 5301 | CAAGTACTTT GACACCACCA TCGACCGGAA GAGGTACACC AGCACCAAAG |
| 5351 | AGGTGCTGGA CGCCACCCTG ATCCACCAGA GCATCACCGG CCTGTACGAG |
| 5401 | ACACGGATCG ACCTGTCTCA GCTGGGAGGC GACAAGCGAC CTGCCGCCAC |
| 5451 | AAAGAAGGCT GGACAGGCTA AGAAGAAGAA AGATTACAAA GACGATGACG |
| 5501 | ATAAGGGATC CGGCGCAACA AACTTCTCTC TGCTGAAACA AGCCGGAGAT |
| 5551 | GTCGAAGAGA ATCCTGGACC GATGGTGTCC AAAGGGGAGG AACTCTTCAC |
| 5601 | TGGCGTTGTC CCAATTCTGG TGGAGCTGGA CGGCGACGTA AATGGCCACA |
| 5651 | AGTTTAGCGT GAGTGGGGAG GGAGAGGGTG ACGCGACATA CGGCAAGCTG |
| 5701 | ACACTGAAAT TTATTTGTAC GACCGGGAAA CTGCCCGTGC CCTGGCCCAC |
| 5751 | ACTTGTGACG ACTTTGACCT ATGGCGTCCA GTGCTTTTCC AGGTATCCAG |
| 5801 | ACCATATGAA GCAGCACGAC TTCTTTAAAA GCGCTATGCC GGAAGGGTAC |
| 5851 | GTTCAGGAGC GCACGATTTT TTTTAAGGAC GATGGTAATT ATAAGACCCG |
| 5901 | AGCCGAGGTT AAATTTGAGG GAGATACCCT GGTGAATCGC ATCGAACTGA |
| 5951 | AGGGCATTGA TTTCAAGGAG GATGGCAATA TTCTCGGCCA CAAACTTGAG |
| 6001 | TACAACTACA ATTCTCACAA CGTATACATC ATGGCGGATA AACAGAAGAA |
| 6051 | CGGAATCAAG GTGAACTTCA AGATTAGGCA CAACATTGAA GATGGCAGCG |
| 6101 | TTCAGCTGGC CGACCACTAT CAACAGAATA CCCCTATTGG GGATGGCCCT |
| 6151 | GTGCTCTTGC CCGATAACCA CTATCTGAGC ACCCAGAGCG CGCTGAGCAA |
| 6201 | AGATCCAAAT GAAAAGCGGG ACCATATGGT GCTGTTGGAG TTTGTCACTG |
| 6251 | CCGCAGGAAT CACACTGGGC ATGGACGAGC TGTACAAGTC TTAACTTGTA |
| 6301 | CAAAGTGGTT GATATCGGTA AGCCTATCCC TAACCCTCTC CTCGGTCTCG |
| 6351 | ATTCTACGTA GTAATGAACT AGTACCGGTT AAGTCGACAA TCAACGCGTT |
| 6401 | AAGTCGACAA TCAACCTCTG GATTACAAAA TTTGTGAAAG ATTGACTGGT |
| 6451 | ATTCTTAACT ATGTTGCTCC TTTTACGCTA TGTGGATACG CTGCTTTAAT |
| 6501 | GCCTTTGTAT CATGCTATTG CTTCCCGTAT GGCTTTCATT TTCTCCTCCT |
| 6551 | TGTATAAATC CTGGTTGCTG TCTCTTTATG AGGAGTTGTG GCCCGTTGTC |
| 6601 | AGGCAACGTG GCGTGGTGTG CACTGTGTTT GCTGACGCAA CCCCCACTGG |
| 6651 | TTGGGGCATT GCCACCACCT GTCAGCTCCT TTCCGGGACT TTCGCTTTCC |
| 6701 | CCCTCCCTAT TGCCACGGCG GAACTCATCG CCGCCTGCCT TGCCCGCTGC |
| 6751 | TGGACAGGGG CTCGGCTGTT GGGCACTGAC AATTCCGTGG TGTTGTCGGG |
| 6801 | GAAATCATCG TCCTTTCCTT GGCTGCTCGC CTGTGTTGCC ACCTGGATTC |
| 6851 | TGCGCGGGAC GTCCTTCTGC TACGTCCCTT CGGCCCTCAA TCCAGCGGAC |
| 6901 | CTTCCTTCCC GCGGCCTGCT GCCGGCTCTG CGGCCTCTTC CGCGTCTTCG |
| 6951 | CCTTCGCCCT CAGACGAGTC GGATCTCCCT TTGGGCCGCC TCCCCGCGTC |
| 7001 | GACTTTAAGA CCAATGACTT ACAAGGCAGC TGTAGATCTT AGCCACTTTT |
| 7051 | TAAAAGAAAA GGGGGGACTG GAAGGGCTAA TTCACTCCCA ACGAAGACAA |
| 7101 | GATGGGATCA ATTCACCATG GGAATAACTT CGTATAGCAT ACATTATACG |
| 7151 | AAGTTATGCT GCTTTTTGCT TGTACTGGGT CTCTCTGGTT AGACCAGATC |
| 7201 | TGAGCCTGGG AGCTCTCTGG CTAACTAGGG AACCCACTGC TTAAGCCTCA |
| 7251 | ATAAAGCTTG CCTTGAGTGC TTCAAGTAGT GTGTGCCCGT CTGTTGTGTG |
| 7301 | ACTCTGGTAA CTAGAGATCC CTCAGACCCT TTTAGTCAGT GTGGAAAATC |
| 7351 | TCTAGCATAC GTATAGTAGT TCATGTCATC TTATTATTCA GTATTTATAA |
| 7401 | CTTGCAAAGA AATGAATATC AGAGAGTGAG AGGAACTTGT TTATTGCAGC |
| 7451 | TTATAATGGT TACAAATAAA GCAATAGCAT CACAAATTTC ACAAATAAAG |
| 7501 | CATTTTTTTC ACTGCATTCT AGTTGTGGTT TGTCCAAACT CATCAATGTA |
| 7551 | TCTTATCATG TCTGGCTCTA GCTATCCCGC CCCTAACTCC GCCCATCCCG |
| 7601 | CCCCTAACTC CGCCCAGTTC CGCCCATTCT CCGCCCCATG GCTGACTAAT |
| 7651 | TTTTTTTATT TATGCAGAGG CCGAGGCCGC CTCGGCCTCT GAGCTATTCC |
| 7701 | AGAAGTAGTG AGGAGGCTTT TTTGGAGGCC TAGGGACGTA CCCAATTCGC |
| 7751 | CCTATAGTGA GTCGTATTAC GCGCGCTCAC TGGCCGTCGT TTTACAACGT |
| 7801 | CGTGACTGGG AAAACCCTGG CGTTACCCAA CTTAATCGCC TTGCAGCACA |
| 7851 | TCCCCCTTTC GCCAGCTGGC GTAATAGCGA AGAGGCCCGC ACCGATCGCC |
| 7901 | CTTCCCAACA GTTGCGCAGC CTGAATGGCG AATGGGACGC GCCCTGTAGC |
| 7951 | GGCGCATTAA GCGCGGCGGG TGTGGTGGTT ACGCGCAGCG TGACCGCTAC |
| 8001 | ACTTGCCAGC GCCCTAGCGC CCGCTCCTTT CGCTTTCTTC CCTTCCTTTC |
| 8051 | TCGCCACGTT CGCCGGCTTT CCCCGTCAAG CTCTAAATCG GGGGCTCCCT |
| 8101 | TTAGGGTTCC GATTTAGTGC TTTACGGCAC CTCGACCCCA AAAAACTTGA |
| 8151 | TTAGGGTGAT GGTTCACGTA GTGGGCCATC GCCCTGATAG ACGGTTTTTC |
| 8201 | GCCCTTTGAC GTTGGAGTCC ACGTTCTTTA ATAGTGGACT CTTGTTCCAA |
| 8251 | ACTGGAACAA CACTCAACCC TATCTCGGTC TATTCTTTTG ATTTATAAGG |
| 8301 | GATTTTGCCG ATTTCGGCCT ATTGGTTAAA AAATGAGCTG ATTTAACAAA |
| 8351 | AATTTAACGC GAATTTTAAC AAAATATTAA CGCTTACAAT TTAGGTGGCA |
| 8401 | CTTTTCGGGG AAATGTGCGC GGAACCCCTA TTTGTTTATT TTTCTAAATA |
| 8451 | CATTCAAATA TGTATCCGCT CATGAGACAA TAACCCTGAT AAATGCTTCA |
| 8501 | ATAATATTGA AAAAGGAAGA GTATGAGTAT TCAACATTTC CGTGTCGCCC |
| 8551 | TTATTCCCTT TTTTGCGGCA TTTTGCCTTC CTGTTTTTGC TCACCCAGAA |
| 8601 | ACGCTGGTGA AAGTAAAAGA TGCTGAAGAT CAGTTGGGTG CACGAGTGGG |
| 8651 | TTACATCGAA CTGGATCTCA ACAGCGGTAA GATCCTTGAG AGTTTTCGCC |
| 8701 | CCGAAGAACG TTTTCCAATG ATGAGCACTT TTAAAGTTCT GCTATGTGGC |
| 8751 | GCGGTATTAT CCCGTATTGA CGCCGGGCAA GAGCAACTCG GTCGCCGCAT |
| 8801 | ACACTATTCT CAGAATGACT TGGTTGAGTA CTCACCAGTC ACAGAAAAGC |
| 8851 | ATCTTACGGA TGGCATGACA GTAAGAGAAT TATGCAGTGC TGCCATAACC |
| 8901 | ATGAGTGATA ACACTGCGGC CAACTTACTT CTGACAACGA TCGGAGGACC |
| 8951 | GAAGGAGCTA ACCGCTTTTT TGCACAACAT GGGGGATCAT GTAACTCGCC |
| 9001 | TTGATCGTTG GGAACCGGAG CTGAATGAAG CCATACCAAA CGACGAGCGT |
| 9051 | GACACCACGA TGCCTGTAGC AATGGCAACA ACGTTGCGCA AACTATTAAC |
| 9101 | TGGCGAACTA CTTACTCTAG CTTCCCGGCA ACAATTAATA GACTGGATGG |
| 9151 | AGGCGGATAA AGTTGCAGGA CCACTTCTGC GCTCGGCCCT TCCGGCTGGC |
| 9201 | TGGTTTATTG CTGATAAATC TGGAGCCGGT GAGCGTGGGT CTCGCGGTAT |
| 9251 | CATTGCAGCA CTGGGGCCAG ATGGTAAGCC CTCCCGTATC GTAGTTATCT |
| 9301 | ACACGACGGG GAGTCAGGCA ACTATGGATG AACGAAATAG ACAGATCGCT |
| 9351 | GAGATAGGTG CCTCACTGAT TAAGCATTGG TAACTGTCAG ACCAAGTTTA |
| 9401 | CTCATATATA CTTTAGATTG ATTTAAAACT TCATTTTTAA TTTAAAAGGA |
| 9451 | TCTAGGTGAA GATCCTTTTT GATAATCTCA TGACCAAAAT CCCTTAACGT |
| 9501 | GAGTTTTCGT TCCACTGAGC GTCAGACCCC GTAGAAAAGA TCAAAGGATC |
| 9551 | TTCTTGAGAT CCTTTTTTTC TGCGCGTAAT CTGCTGCTTG CAAACAAAAA |
| 9601 | AACCACCGCT ACCAGCGGTG GTTTGTTTGC CGGATCAAGA GCTACCAACT |
| 9651 | CTTTTTCCGA AGGTAACTGG CTTCAGCAGA GCGCAGATAC CAAATACTGT |
| 9701 | TCTTCTAGTG TAGCCGTAGT TAGGCCACCA CTTCAAGAAC TCTGTAGCAC |
| 9751 | CGCCTACATA CCTCGCTCTG CTAATCCTGT TACCAGTGGC TGCTGCCAGT |
| 9801 | GGCGATAAGT CGTGTCTTAC CGGGTTGGAC TCAAGACGAT AGTTACCGGA |
| 9851 | TAAGGCGCAG CGGTCGGGCT GAACGGGGGG TTCGTGCACA CAGCCCAGCT |
| 9901 | TGGAGCGAAC GACCTACACC GAACTGAGAT ACCTACAGCG TGAGCTATGA |
| 9951 | GAAAGCGCCA CGCTTCCCGA AGGGAGAAAG GCGGACAGGT ATCCGGTAAG |
| 10001 | CGGCAGGGTC GGAACAGGAG AGCGCACGAG GGAGCTTCCA GGGGGAAACG |
| 10051 | CCTGGTATCT TTATAGTCCT GTCGGGTTTC GCCACCTCTG ACTTGAGCGT |
| 10101 | CGATTTTTGT GATGCTCGTC AGGGGGGCGG AGCCTATGGA AAAACGCCAG |
| 10151 | CAACGCGGCC TTTTTACGGT TCCTGGCCTT TTGCTGGCCT TTTGCTCACA |
| 10201 | TGTTCTTTCC TGCGTTATCC CCTGATTCTG TGGATAACCG TATTACCGCC |
| 10251 | TTTGAGTGAG CTGATACCGC TCGCCGCAGC CGAACGACCG AGCGCAGCGA |
| 10301 | GTCAGTGAGC GAGGAAGCGG AAGAGCGCCC AATACGCAAA CCGCCTCTCC |
| 10351 | CCGCGCGTTG GCCGATTCAT TAATGCAGCT GGCACGACAG GTTTCCCGAC |
| 10401 | TGGAAAGCGG GCAGTGAGCG CAACGCAATT AATGTGAGTT AGCTCACTCA |
| 10451 | TTAGGCACCC CAGGCTTTAC ACTTTATGCT TCCGGCTCGT ATGTTGTGTG |
| 10501 | GAATTGTGAG CGGATAACAA TTTCACACAG GAAACAGCTA TGACCATGAT |
| 10551 | TACGCCAAGC GCGCAATTAA CCCTCACTAA AGGGAACAAA AGCTGGAGCT |
| 10601 | GCAAGCTTAA TGTAGTCTTA TGCAATACTC TTGTAGTCTT GCAACATGGT |
| 10651 | AACGATGAGT TAGCAACATG CCTTACAAGG AGAGAAAAAG CACCGTGCAT |
| 10701 | GCCGATTGGT GGAAGTAAGG TGGTACGATC GTGCCTTATT AGGAAGGCAA |
| 10751 | CAGACGGGTC TGACATGGAT TGGACGAACC ACTGAATTGC CGCATTGCAG |
| 10801 | AGATATTGTA TTTAAGTGCC TAGCTCGATA CATAAACGGG TCTCTCTGGT |
| 10851 | TAGACCAGAT CTGAGCCTGG GAGCTCTCTG GCTAACTAGG GAACCCACTG |
| 10901 | CTTAAGCCTC AATAAAGCTT GCCTTGAGTG CTTCAAGTAG TGTGTGCCCG |
| 10951 | TCTGTTGTGT GACTCTGGTA ACTAGAGATC CCTCAGACCC TTTTAGTCAG |
| 11001 | TGTGGAAAAT CTCTAGCAGT GGCGCCCGAA CAGGGACTTG AAAGCGAAAG |
| 11051 | GGAAACCAGA GGAGCTCTCT CGACGCAGGA CTCGGCTTGC TGAAGCGCGC |
| 11101 | ACGGCAAGAG GCGAGGGGCG GCGACTGGTG AGTACGCCAA AAATTTTGAC |
| 11151 | TAGCGGAGGC TAGAAGGAGA GAGATGGGTG CGAGAGCGTC AGTATTAAGC |
| 11201 | GGGGGAGAAT TAGATCGCGA TGGGAAAAAA TTCGGTTAAG GCCAGGGGGA |
| 11251 | AAGAAAAAAT ATAAATTAAA ACATATAGTA TGGGCAAGCA GGGAGCTAGA |
| 11301 | ACGATTCGCA GTTAATCCTG GCCTGTTAGA AACATCAGAA GGCTGTAGAC |
| 11351 | AAATACTGGG ACAGCTACAA CCATCCCTTC AGACAGGATC AGAAGAACTT |
| 11401 | AGATCATTAT ATAATACAGT AGCAACCCTC TATTGTGTGC ATCAAAGGAT |
| 11451 | AGAGATAAAA GACACCAAGG AAGCTTTAGA CAAGATAGAG GAAGAGCAAA |
| 11501 | ACAAAAGTAA GACCACCGCA CAGCAAGCGG CCGCTGATCT TCAGACCTGG |
| 11551 | AGGAGGAGAT ATGAGGGACA ATTGGAGAAG TGAATTATAT AAATATAAAG |
| 11601 | TAGTAAAAAT TGAACCATTA GGAGTAGCAC CCACCAAGGC AAAGAGAAGA |
| 11651 | GTGGTGCAGA GAGAAAAAAG AGCAGTGGGA ATAGGAGCTT TGTTCCTTGG |
| 11701 | GTTCTTGGGA GCAGCAGGAA GCACTATGGG CGCAGCGTCA ATGACGCTGA |
| 11751 | CGGTACAGGC CAGACAATTA TTGTCTGGTA TAGTGCAGCA GCAGAACAAT |
| 11801 | TTGCTGAGGG CTATTGAGGC GCAACAGCAT CTGTTGCAAC TCACAGTCTG |
| 11851 | GGGCATCAAG CAGCTCCAGG CAAGAATCCT GGCTGTGGAA AGATACCTAA |
| 11901 | AGGATCAACA GCTCCTGGGG ATTTGGGGTT GCTCTGGAAA ACTCATTTGC |
| 11951 | ACCACTGCTG TGCCTTGGAA TGCTAGTTGG AGTAATAAAT CTCTGGAACA |
| 12001 | GATTTGGAAT CACACGACCT GGATGGAGTG GGACAGAGAA ATTAACAATT |
| 12051 | ACACAAGCTT AATACACTCC TTAATTGAAG AATCGCAAAA CCAGCAAGAA |
| 12101 | AAGAATGAAC AAGAATTATT GGAATTAGAT AAATGGGCAA GTTTGTGGAA |
| 12151 | TTGGTTTAAC ATAACAAATT GGCTGTGGTA TATAAAATTA TTCATAATGA |
| 12201 | TAGTAGGAGG CTTGGTAGGT TTAAGAATAG TTTTTGCTGT ACTTTCTATA |
| 12251 | GTGAATAGAG TTAGGCAGGG ATATTCACCA TTATCGTTTC AGACCCACCT |
| 12301 | CCCAACCCCG AGGGGACCCA TGCATTGCAT CTCAATTAGT CAGCAACCAG |
| 12351 | GTGTGGAAAG TCCCCAGGCT CCCCAGCAGG CAGAAGTATG CAAAGCATGC |
| 12401 | GTCTCAATTA GTCAGCAACC ATAGTCCCGC CCCTAACTCC GCCCATCCCG |
| 12451 | CCCCTAACTC CGCCCAGTTC CGCCCATTCT CCGCCCCATG GCTGACTAAT |
| 12501 | TTTTTTTATT TATGCAGAGG CCGAGGCCGC CTCGGCCTCT GAGCTATTCC |
| 12551 | AGAAGTAGTG AGGAGGCTTT TTTGGAGGCC TAGGCTTTTG CAAAAAGCTT |
| 12601 | TCTAGAGGTA CCACCATGGC CAAGCCTTTG TCTCAAGAAG AATCCACCCT |
| 12651 | CATTGAAAGA GCAACGGCTA CAATCAACAG CATCCCCATC TCTGAAGACT |
| 12701 | ACAGCGTCGC CAGCGCAGCT CTCTCTAGCG ACGGCCGCAT CTTCACTGGT |
| 12751 | GTCAATGTAT ATCATTTTAC TGGGGGACCT TGTGCAGAAC TCGTGGTGCT |
| 12801 | GGGCACTGCT GCTGCTGCGG CAGCTGGCAA CCTGACTTGT ATCGTCGCGA |
| 12851 | TCGGAAATGA GAACAGGGGC ATCTTGAGCC CCTGCGGACG GTGCCGACAG |
| 12901 | GTGCTTCTCG ATCTGCATCC TGGGATCAAA GCCATAGTGA AGGACAGTGA |
| 12951 | TGGACAGCCG ACGGCAGTTG GGATTCGTGA ATTGCTGCCC TCTGGTTATG |
| 13001 | TGTGGGAGGG CCTGCAGCTG CAGTAGTAAG GCGCGCCGTT AACGAATTCT |
| 13051 | AGATCTTGAG ACAAATGGCA GTATTCATCC ACAATTTTAA AAGAAAAGGG |
| 13101 | GGGATTGGGG GGTACAGTGC AGGGGAAAGA ATAGTAGACA TAATAGCAAC |
| 13151 | AGACATACAA ACTAAAGAAT TACAAAAACA AATTACAAAA ATTCAAAATT |
| 13201 | TTCGGGTTTA TTACAGGGAC AGCAGAGATC CACTTTGGCG CCGG |

### ANNOTATIONS

16-1275: EF1α (promoter)
1294-1298: attR1
1330-5433: S. Pyogenes Cas9
5434-5481: NLS (nucleoplasmin)
5482-5505: FLAG
5506-5571: P2A
5572-6291: EGFP
6295-6309: attR2
6317-6358: V5
6409-6997: WPRE
7052-7067: cPPT
7124-7157: loxP
7177-7357: HIV-1 5′ LTR
7434-7564: SV40 polyodenylation signal
7644-7719: SV40 origin of replication
7937-8329: F1 ori
8523-9383: AmpR
9531-10198: pUC ori
10607-11-19: 5′LTR
11070-11207: psi
11174-11538: gag
11684-11925: Rev response element (RRE)
12326-12600: SV40 (promoter)
12613-13029: BlastR
13085-13202: cPPT

| mKate sgRNA lox2272: | |
|---|---|
| (SEQ ID NO: 8) | |
| 1 | GATCGCCCTT CCCAACAGTT GCGCAGCCTG AATGGCGAAT GGGACGCGCC |
| 51 | CTGTAGCGGC GCATTAAGCG CGGCGGGTGT GGTGGTTACG CGCAGCGTGA |
| 101 | CCGCTACACT TGCCAGCGCC CTAGCGCCCG CTCCTTTCGC TTTCTTCCCT |
| 151 | TCCTTTCTCG CCACGTTCGC CGGCTTTCCC CGTCAAGCTC TAAATCGGGG |
| 201 | GCTCCCTTTA GGGTTCCGAT TTAGTGCTTT ACGGCACCTC GACCCCAAAA |
| 251 | AACTTGATTA GGGTGATGGT TCACGTAGTG GGCCATCGCC CTGATAGACG |
| 301 | GTTTTTCGCC CTTTGACGTT GGAGTCCACG TTCTTTAATA GTGGACTCTT |
| 351 | GTTCCAAACT GGAACAACAC TCAACCCTAT CTCGGTCTAT TCTTTTGATT |
| 401 | TATAAGGGAT TTTGCCGATT TCGGCCTATT GGTTAAAAAA TGAGCTGATT |
| 451 | TAACAAAAAT TTAACGCGAA TTTTAACAAA ATATTAACGC TTACAATTTA |
| 501 | GGTGGCACTT TTCGGGGAAA TGTGCGCGGA ACCCCTATTT GTTTATTTTT |
| 551 | CTAAATACAT TCAAATATGT ATCCGCTCAT GAGACAATAA CCCTGATAAA |
| 601 | TGCTTCAATA ATATTGAAAA AGGAAGAGTA TGAGTATTCA ACATTTCCGT |
| 651 | GTCGCCCTTA TTCCCTTTTT TGCGGCATTT TGCCTTCCTG TTTTTGCTCA |
| 701 | CCCAGAAACG CTGGTGAAAG TAAAAGATGC TGAAGATCAG TTGGGTGCAC |
| 751 | GAGTGGGTTA CATCGAACTG GATCTCAACA GCGGTAAGAT CCTTGAGAGT |
| 801 | TTTCGCCCCG AAGAACGTTT TCCAATGATG AGCACTTTTA AAGTTCTGCT |
| 851 | ATGTGGCGCG GTATTATCCC GTATTGACGC CGGGCAAGAG CAACTCGGTC |
| 901 | GCCGCATACA CTATTCTCAG AATGACTTGG TTGAGTACTC ACCAGTCACA |
| 951 | GAAAAGCATC TTACGGATGG CATGACAGTA AGAGAATTAT GCAGTGCTGC |
| 1001 | CATAACCATG AGTGATAACA CTGCGGCCAA CTTACTTCTG ACAACGATCG |
| 1051 | GAGGACCGAA GGAGCTAACC GCTTTTTTGC ACAACATGGG GGATCATGTA |
| 1101 | ACTCGCCTTG ATCGTTGGGA ACCGGAGCTG AATGAAGCCA TACCAAACGA |
| 1151 | CGAGCGTGAC ACCACGATGC CTGTAGCAAT GGCAACAACG TTGCGCAAAC |
| 1201 | TATTAACTGG CGAACTACTT ACTCTAGCTT CCCGGCAACA ATTAATAGAC |
| 1251 | TGGATGGAGG CGGATAAAGT TGCAGGACCA CTTCTGCGCT CGGCCCTTCC |
| 1301 | GGCTGGCTGG TTTATTGCTG ATAAATCTGG AGCCGGTGAG CGTGGGTCTC |
| 1351 | GCGGTATCAT TGCAGCACTG GGGCCAGATG GTAAGCCCTC CCGTATCGTA |
| 1401 | GTTATCTACA CGACGGGGAG TCAGGCAACT ATGGATGAAC GAAATAGACA |
| 1451 | GATCGCTGAG ATAGGTGCCT CACTGATTAA GCATTGGTAA CTGTCAGACC |
| 1501 | AAGTTTACTC ATATATACTT TAGATTGATT TAAAACTTCA TTTTTAATTT |
| 1551 | AAAAGGATCT AGGTGAAGAT CCTTTTTGAT AATCTCATGA CCAAAATCCC |
| 1601 | TTAACGTGAG TTTTCGTTCC ACTGAGCGTC AGACCCCGTA GAAAAGATCA |
| 1651 | AAGGATCTTC TTGAGATCCT TTTTTTCTGC GCGTAATCTG CTGCTTGCAA |
| 1701 | ACAAAAAAAC CACCGCTACC AGCGGTGGTT TGTTTGCCGG ATCAAGAGCT |
| 1751 | ACCAACTCTT TTTCCGAAGG TAACTGGCTT CAGCAGAGCG CAGATACCAA |
| 1801 | ATACTGTTCT TCTAGTGTAG CCGTAGTTAG GCCACCACTT CAAGAACTCT |
| 1851 | GTAGCACCGC CTACATACCT CGCTCTGCTA ATCCTGTTAC CAGTGGCTGC |
| 1901 | TGCCAGTGGC GATAAGTCGT GTCTTACCGG GTTGGACTCA AGACGATAGT |
| 1951 | TACCGGATAA GGCGCAGCGG TCGGGCTGAA CGGGGGGTTC GTGCACACAG |
| 2001 | CCCAGCTTGG AGCGAACGAC CTACACCGAA CTGAGATACC TACAGCGTGA |
| 2051 | GCTATGAGAA AGCGCCACGC TTCCCGAAGG GAGAAAGGCG GACAGGTATC |
| 2101 | CGGTAAGCGG CAGGGTCGGA ACAGGAGAGC GCACGAGGGA GCTTCCAGGG |
| 2151 | GGAAACGCCT GGTATCTTTA TAGTCCTGTC GGGTTTCGCC ACCTCTGACT |
| 2201 | TGAGCGTCGA TTTTTGTGAT GCTCGTCAGG GGGGCGGAGC CTATGGAAAA |
| 2251 | ACGCCAGCAA CGCGGCCTTT TTACGGTTCC TGGCCTTTTG CTGGCCTTTT |
| 2301 | GCTCACATGT TCTTTCCTGC GTTATCCCCT GATTCTGTGG ATAACCGTAT |
| 2351 | TACCGCCTTT GAGTGAGCTG ATACCGCTCG CCGCAGCCGA ACGACCGAGC |
| 2401 | GCAGCGAGTC AGTGAGCGAG GAAGCGGAAG AGCGCCCAAT ACGCAAACCG |
| 2451 | CCTCTCCCCG CGCGTTGGCC GATTCATTAA TGCAGCTGGC ACGACAGGTT |
| 2501 | TCCCGACTGG AAAGCGGGCA GTGAGCGCAA CGCAATTAAT GTGAGTTAGC |
| 2551 | TCACTCATTA GGCACCCCAG GCTTTACACT TTATGCTTCC GGCTCGTATG |
| 2601 | TTGTGTGGAA TTGTGAGCGG ATAACAATTT CACACAGGAA ACAGCTATGA |
| 2651 | CCATGATTAC GCCAAGCGCG CAATTAACCC TCACTAAAGG GAACAAAAGC |
| 2701 | TGGAGCTGCA AGCTTAATGT AGTCTTATGC AATACTCTTG TAGTCTTGCA |
| 2751 | ACATGGTAAC GATGAGTTAG CAACATGCCT TACAAGGAGA GAAAAAGCAC |
| 2801 | CGTGCATGCC GATTGGTGGA AGTAAGGTGG TACGATCGTG CCTTATTAGG |
| 2851 | AAGGCAACAG ACGGGTCTGA CATGGATTGG ACGAACCACT GAATTGCCGC |
| 2901 | ATTGCAGAGA TATTGTATTT AAGTGCCTAG CTCGATACAT AAACGGGTCT |
| 2951 | CTCTGGTTAG ACCAGATCTG AGCCTGGGAG CTCTCTGGCT AACTAGGGAA |
| 3001 | CCCACTGCTT AAGCCTCAAT AAAGCTTGCC TTGAGTGCTT CAAGTAGTGT |
| 3051 | GTGCCCGTCT GTTGTGTGAC TCTGGTAACT AGAGATCCCT CAGACCCTTT |
| 3101 | TAGTCAGTGT GGAAAATCTC TAGCAGTGGC GCCCGAACAG GGACTTGAAA |
| 3151 | GCGAAAGGGA AACCAGAGGA GCTCTCTCGA CGCAGGACTC GGCTTGCTGA |
| 3201 | AGCGCGCACG GCAAGAGGCG AGGGGCGGCG ACTGGTGAGT ACGCCAAAAA |
| 3251 | TTTTGACTAG CGGAGGCTAG AAGGAGAGAG ATGGGTGCGA GAGCGTCAGT |
| 3301 | ATTAAGCGGG GGAGAATTAG ATCGCGATGG GAAAAAATTC GGTTAAGGCC |
| 3351 | AGGGGGAAAG AAAAAATATA AATTAAAACA TATAGTATGG GCAAGCAGGG |
| 3401 | AGCTAGAACG ATTCGCAGTT AATCCTGGCC TGTTAGAAAC ATCAGAAGGC |
| 3451 | TGTAGACAAA TACTGGGACA GCTACAACCA TCCCTTCAGA CAGGATCAGA |
| 3501 | AGAACTTAGA TCATTATATA ATACAGTAGC AACCCTCTAT TGTGTGCATC |
| 3551 | AAAGGATAGA GATAAAAGAC ACCAAGGAAG CTTTAGACAA GATAGAGGAA |
| 3601 | GAGCAAAACA AAAGTAAGAC CACCGCACAG CAAGCGGCCG CTGATCTTCA |
| 3651 | GACCTGGAGG AGGAGATATG AGGGACAATT GGAGAAGTGA ATTATATAAA |
| 3701 | TATAAAGTAG TAAAAATTGA ACCATTAGGA GTAGCACCCA CCAAGGCAAA |
| 3751 | GAGAAGAGTG GTGCAGAGAG AAAAAAGAGC AGTGGGAATA GGAGCTTTGT |
| 3801 | TCCTTGGGTT CTTGGGAGCA GCAGGAAGCA CTATGGGCGC AGCGTCAATG |
| 3851 | ACGCTGACGG TACAGGCCAG ACAATTATTG TCTGGTATAG TGCAGCAGCA |
| 3901 | GAACAATTTG CTGAGGGCTA TTGAGGCGCA ACAGCATCTG TTGCAACTCA |
| 3951 | CAGTCTGGGG CATCAAGCAG CTCCAGGCAA GAATCCTGGC TGTGGAAAGA |
| 4001 | TACCTAAAGG ATCAACAGCT CCTGGGGATT TGGGGTTGCT CTGGAAAACT |
| 4051 | CATTTGCACC ACTGCTGTGC CTTGGAATGC TAGTTGGAGT AATAAATCTC |
| 4101 | TGGAACAGAT TTGGAATCAC ACGACCTGGA TGGAGTGGGA CAGAGAAATT |
| 4151 | AACAATTACA CAAGCTTAAT ACACTCCTTA ATTGAAGAAT CGCAAAACCA |
| 4201 | GCAAGAAAAG AATGAACAAG AATTATTGGA ATTAGATAAA TGGGCAAGTT |
| 4251 | TGTGGAATTG GTTTAACATA ACAAATTGGC TGTGGTATAT AAAATTATTC |
| 4301 | ATAATGATAG TAGGAGGCTT GGTAGGTTTA AGAATAGTTT TTGCTGTACT |
| 4351 | TTCTATAGTG AATAGAGTTA GGCAGGGATA TTCACCATTA TCGTTTCAGA |
| 4401 | CCCACCTCCC AACCCCGAGG GGACCCGGTA CCGAGGGCCT ATTTCCCATG |
| 4451 | ATTCCTTCAT ATTTGCATAT ACGATACAAG GCTGTTAGAG AGATAATTAG |
| 4501 | AATTAATTTG ACTGTAAACA CAAAGATATT AGTACAAAAT ACGTGACGTA |
| 4551 | GAAAGTAATA ATTTCTTGGG TAGTTTGCAG TTTTAAAATT ATGTTTTAAA |
| 4601 | ATGGACTATC ATATGCTTAC CGTAACTTGA AAGTATTTCG ATTTCTTGGC |
| 4651 | TTTATATATC TTGTGGAAAG GACGAAACAC CGGAGACGCT TTTTTCGTCT |
| 4701 | CAGTTTGAGA GCTAGAAATA GCAAGTTCAA ATAAGGCTAG TCCGTTATCA |
| 4751 | ACTTGAAAAA GTGGCACCGA GTCGGTGCTT TTTTGAATTC AAGCTTGGCG |
| 4801 | TAACTAGATC TTGAGACAAA TGGCAGTATT CATCCACAAT TTTAAAAGAA |
| 4851 | AAGGGGGGAT TGGGGGGTAC AGTGCAGGGG AAAGAATAGT AGACATAATA |
| 4901 | GCAACAGACA TACAAACTAA AGAATTACAA AAACAAATTA CAAAAATTCA |
| 4951 | AAATTTTCGG GTTTATTACA GGGACAGCAG AGATCCACTT TGGCGCCGGC |
| 5001 | TCGAGGGGGC CCGGGATAAC TTCGTATAGT ACACATTATA CGAAGTTATT |
| 5051 | GCAAAGATGG ATAAAGTTTT AAACAGAGAG GAATCTTTGC AGCTAATGGA |
| 5101 | CCTTCTAGGT CTTGAAAGGA GTGGGAATTG GCTCCGGTGC CCGTCAGTGG |
| 5151 | GCAGAGCGCA CATCGCCCAC AGTCCCCGAG AAGTTGGGGG GAGGGGTCGG |
| 5201 | CAATTGATCC GGTGCCTAGA GAAGGTGGCG CGGGGTAAAC TGGGAAAGTG |
| 5251 | ATGTCGTGTA CTGGCTCCGC CTTTTTCCCG AGGGTGGGGG AGAACCGTAT |
| 5301 | ATAAGTGCAG TAGTCGCCGT GAACGTTCTT TTTCGCAACG GGTTTGCCGC |
| 5351 | CAGAACACAG GTAAGTGCCG TGTGTGGTTC CCGCGGGCCT GGCCTCTTTA |
| 5401 | CGGGTTATGG CCCTTGCGTG CCTTGAATTA CTTCCACCTG GCTGCAGTAC |
| 5451 | GTGATTCTTG ATCCCGAGCT TCGGGTTGGA AGTGGGTGGG AGAGTTCGAG |
| 5501 | GCCTTGCGCT TAAGGAGCCC CTTCGCCTCG TGCTTGAGTT GAGGCCTGGC |
| 5551 | CTGGGCGCTG GGGCCGCCGC GTGCGAATCT GGTGGCACCT TCGCGCCTGT |
| 5601 | CTCGCTGCTT TCGATAAGTC TCTAGCCATT TAAAATTTTT GATGACCTGC |
| 5651 | TGCGACGCTT TTTTTCTGGC AAGATAGTCT TGTAAATGCG GGCCAAGATC |
| 5701 | TGCACACTGG TATTTCGGTT TTTGGGGCCG CGGGCGGCGA CGGGGCCCGT |
| 5751 | GCGTCCCAGC GCACATGTTC GGCGAGGCGG GGCCTGCGAG CGCGGCCACC |
| 5801 | GAGAATCGGA CGGGGGTAGT CTCAAGCTGG CCGGCCTGCT CTGGTGCCTG |
| 5851 | GCCTCGCGCC GCCGTGTATC GCCCCGCCCT GGGCGGCAAG GCTGGCCCGG |
| 5901 | TCGGCACCAG TTGCGTGAGC GGAAAGATGG CCGCTTCCCG GCCCTGCTGC |
| 5951 | AGGGAGCTCA AAATGGAGGA CGCGGCGCTC GGGAGAGCGG GCGGGTGAGT |
| 6001 | CACCCACACA AAGGAAAAGG GCCTTTCCGT CCTCAGCCGT CGCTTCATGT |
| 6051 | GACTCCACGG AGTACCGGGC GCCGTCCAGG CACCTCGATT AGTTCTCGAG |
| 6101 | CTTTTGGAGT ACGTCGTCTT TAGGTTGGGG GGAGGGGTTT TATGCGATGG |
| 6151 | AGTTTCCCCA CACTGAGTGG GTGGAGACTG AAGTTAGGCC AGCTTGGCAC |
| 6201 | TTGATGTAAT TCTCCTTGGA ATTTGCCCTT TTTGAGTTTG GATCTTGGTT |
| 6251 | CATTCTCAAG CCTCAGACAG TGGTTCAAAG TTTTTTTCTT CCATTTCAGG |
| 6301 | TGTCGTGACG TACGGCCACC ATGACCGAGT ACAAGCCCAC GGTGCGCCTC |
| 6351 | GCCACCCGCG ACGACGTCCC CAGGGCCGTA CGCACCCTCG CCGCCGCGTT |
| 6401 | CGCCGACTAC CCCGCCACGC GCCACACCGT CGATCCGGAC CGCCACATCG |
| 6451 | AGCGGGTCAC CGAGCTGCAA GAACTCTTCC TCACGCGCGT CGGGCTCGAC |
| 6501 | ATCGGCAAGG TGTGGGTCGC GGACGACGGC GCCGCCGTGG CGGTCTGGAC |
| 6551 | CACGCCGGAG AGCGTCGAAG CGGGGGCGGT GTTCGCCGAG ATCGGCCCGC |
| 6601 | GCATGGCCGA GTTGAGCGGT TCCCGGCTGG CCGCGCAGCA ACAGATGGAA |
| 6651 | GGCCTCCTGG CGCCGCACCG GCCCAAGGAG CCCGCGTGGT TCCTGGCCAC |
| 6701 | CGTCGGCGTT TCGCCCGACC ACCAGGGCAA GGGTCTGGGC AGCGCCGTCG |
| 6751 | TGCTCCCCGG AGTGGAGGCG GCCGAGCGCG CCGGGGTGCC CGCCTTCCTG |
| 6801 | GAGACCTCCG CGCCCCGCAA CCTCCCCTTC TACGAGCGGC TCGGCTTCAC |
| 6851 | CGTCACCGCC GACGTCGAGG TGCCCGAAGG ACCGCGCACC TGGTGCATGA |
| 6901 | CCCGCAAGCC CGGTGCCGCT AGCCTGCAGG GATCCGGCGC AACAAACTTC |
| 6951 | TCTCTGCTGA AACAAGCCGG AGATGTCGAA GAGAATCCTG GACCGGCTAG |
| 7001 | CATGGTGAGC GAGCTGATTA AGGAGAACAT GCACATGAAG CTGTACATGG |
| 7051 | AGGGCACCGT GAACAACCAC CACTTCAAGT GCACATCCGA GGGCGAAGGC |
| 7101 | AAGCCCTACG AGGGCACCCA GACCATGAGA ATCAAGGCGG TCGAGGGCGG |
| 7151 | CCCTCTCCCC TTCGCCTTCG ACATCCTGGC TACCAGCTTC ATGTACGGCA |
| 7201 | GCAAAACCTT CATCAACCAC ACCCAGGGCA TCCCCGACTT CTTTAAGCAG |
| 7251 | TCCTTCCCCG AGGGCTTCAC ATGGGAGAGA GTCACCACAT ACGAAGACGG |
| 7301 | GGGCGTGCTG ACCGCTACCC AGGACACCAG CCTCCAGGAC GGCTGCCTCA |
| 7351 | TCTACAACGT CAAGATCAGA GGGGTGAACT TCCCATCCAA CGGCCCTGTG |
| 7401 | ATGCAGAAGA AAACACTCGG CTGGGAGGCC TCCACCGAGA CCCTGTACCC |
| 7451 | CGCTGACGGC GGCCTGGAAG GCAGAGCCGA CATGGCCCTG AAGCTCGTGG |
| 7501 | GCGGGGGCCA CCTGATCTGC AACTTGAAGA CCACATACAG ATCCAAGAAA |
| 7551 | CCCGCTAAGA ACCTCAAGAT GCCCGGCGTC TACTATGTGG ACAGAAGACT |
| 7601 | GGAAAGAATC AAGGAGGCCG ACAAAGAGAC CTACGTCGAG CAGCACGAGG |
| 7651 | TGGCTGTGGC CAGATACTGC GACCTCCCTA GCAAACTGGG GCACAGATAA |
| 7701 | ATAACTTCGT ATAGTACACA TTATACGAAG TTATACGCGT TAAGTCGACA |
| 7751 | ATCAACCTCT GGATTACAAA ATTTGTGAAA GATTGACTGG TATTCTTAAC |
| 7801 | TATGTTGCTC CTTTTACGCT ATGTGGATAC GCTGCTTTAA TGCCTTTGTA |
| 7851 | TCATGCTATT GCTTCCCGTA TGGCTTTCAT TTTCTCCTCC TTGTATAAAT |
| 7901 | CCTGGTTGCT GTCTCTTTAT GAGGAGTTGT GGCCCGTTGT CAGGCAACGT |
| 7951 | GGCGTGGTGT GCACTGTGTT TGCTGACGCA ACCCCCACTG GTTGGGGCAT |
| 8001 | TGCCACCACC TGTCAGCTCC TTTCCGGGAC TTTCGCTTTC CCCCTCCCTA |
| 8051 | TTGCCACGGC GGAACTCATC GCCGCCTGCC TTGCCCGCTG CTGGACAGGG |
| 8101 | GCTCGGCTGT TGGGCACTGA CAATTCCGTG GTGTTGTCGG GGAAATCATC |
| 8151 | GTCCTTTCCT TGGCTGCTCG CCTGTGTTGC CACCTGGATT CTGCGCGGGA |
| 8201 | CGTCCTTCTG CTACGTCCCT TCGGCCCTCA ATCCAGCGGA CCTTCCTTCC |
| 8251 | CGCGGCCTGC TGCCGGCTCT GCGGCCTCTT CCGCGTCTTC GCCTTCGCCC |
| 8301 | TCAGACGAGT CGGATCTCCC TTTGGGCCGC CTCCCCGCGT CGACTTTAAG |
| 8351 | ACCAATGACT TACAAGGCAG CTGTAGATCT TAGCCACTTT TTAAAAGAAA |
| 8401 | AGGGGGGACT GGAAGGGCTA ATTCACTCCC AACGAAGACA AGATCTGCTT |
| 8451 | TTTGCTTGTA CTGGGTCTCT CTGGTTAGAC CAGATCTGAG CCTGGGAGCT |
| 8501 | CTCTGGCTAA CTAGGGAACC CACTGCTTAA GCCTCAATAA AGCTTGCCTT |
| 8551 | GAGTGCTTCA AGTAGTGTGT GCCCGTCTGT TGTGTGACTC TGGTAACTAG |
| 8601 | AGATCCCTCA GACCCTTTTA GTCAGTGTGG AAAATCTCTA GCAGTACGTA |
| 8651 | TAGTAGTTCA TGTCATCTTA TTATTCAGTA TTTATAACTT GCAAAGAAAT |
| 8701 | GAATATCAGA GAGTGAGAGG AACTTGTTTA TTGCAGCTTA TAATGGTTAC |
| 8751 | AAATAAAGCA ATAGCATCAC AAATTTCACA AATAAAGCAT TTTTTTCACT |
| 8801 | GCATTCTAGT TGTGGTTTGT CCAAACTCAT CAATGTATCT TATCATGTCT |
| 8851 | GGCTCTAGCT ATCCCGCCCC TAACTCCGCC CATCCCGCCC CTAACTCCGC |
| 8901 | CCAGTTCCGC CCATTCTCCG CCCCATGGCT GACTAATTTT TTTTATTTAT |
| 8951 | GCAGAGGCCG AGGCCGCCTC GGCCTCTGAG CTATTCCAGA AGTAGTGAGG |
| 9001 | AGGCTTTTTT GGAGGCCTAG GGACGTACCC AATTCGCCCT ATAGTGAGTC |
| 9051 | GTATTACGCG CGCTCACTGG CCGTCGTTTT ACAACGTCGT GACTGGGAAA |
| 9101 | ACCCTGGCGT TACCCAACTT AATCGCCTTG CAGCACATCC CCCTTTCGCC |
| 9151 | AGCTGGCGTA ATAGCGAAGA GGCCCGCACC |

### ANNOTATIONS

44-499: F1 ori
630-1490: AmpR
1638-2305: pUC ori
2714-3126: 5′ LTR
3177-3314: psi
3281-3645: gag
3791-4032: Rev response element (RRE)
4433-4673: U6 (promoter)
4703-4778: sgRNA scaffold
4840-4957: cPPT/CTS
5016-5049: lox2272
5050-6308: EF1α (promoter)
6321-6923: PuroR
6930-6995: P2A
7002-7700: mKate
7701-7734: lox2272
7750-8338: WPRE
8409-8644: 3′ LTR (SIN)
8721-8851: SV40 polyadenylation signal
8932-9006: SV40 origin of replication

| mKate sgRNA lox5171: | |
|---|---|
| (SEQ ID NO: 9) | |
| 1 | GATCGCCCTT CCCAACAGTT GCGCAGCCTG AATGGCGAAT GGGACGCGCC |
| 51 | CTGTAGCGGC GCATTAAGCG CGGCGGGTGT GGTGGTTACG CGCAGCGTGA |
| 101 | CCGCTACACT TGCCAGCGCC CTAGCGCCCG CTCCTTTCGC TTTCTTCCCT |
| 151 | TCCTTTCTCG CCACGTTCGC CGGCTTTCCC CGTCAAGCTC TAAATCGGGG |
| 201 | GCTCCCTTTA GGGTTCCGAT TTAGTGCTTT ACGGCACCTC GACCCCAAAA |
| 251 | AACTTGATTA GGGTGATGGT TCACGTAGTG GGCCATCGCC CTGATAGACG |
| 301 | GTTTTTCGCC CTTTGACGTT GGAGTCCACG TTCTTTAATA GTGGACTCTT |
| 351 | GTTCCAAACT GGAACAACAC TCAACCCTAT CTCGGTCTAT TCTTTTGATT |
| 401 | TATAAGGGAT TTTGCCGATT TCGGCCTATT GGTTAAAAAA TGAGCTGATT |
| 451 | TAACAAAAAT TTAACGCGAA TTTTAACAAA ATATTAACGC TTACAATTTA |
| 501 | GGTGGCACTT TTCGGGGAAA TGTGCGCGGA ACCCCTATTT GTTTATTTTT |
| 551 | CTAAATACAT TCAAATATGT ATCCGCTCAT GAGACAATAA CCCTGATAAA |
| 601 | TGCTTCAATA ATATTGAAAA AGGAAGAGTA TGAGTATTCA ACATTTCCGT |
| 651 | GTCGCCCTTA TTCCCTTTTT TGCGGCATTT TGCCTTCCTG TTTTTGCTCA |
| 701 | CCCAGAAACG CTGGTGAAAG TAAAAGATGC TGAAGATCAG TTGGGTGCAC |
| 751 | GAGTGGGTTA CATCGAACTG GATCTCAACA GCGGTAAGAT CCTTGAGAGT |
| 801 | TTTCGCCCCG AAGAACGTTT TCCAATGATG AGCACTTTTA AAGTTCTGCT |
| 851 | ATGTGGCGCG GTATTATCCC GTATTGACGC CGGGCAAGAG CAACTCGGTC |
| 901 | GCCGCATACA CTATTCTCAG AATGACTTGG TTGAGTACTC ACCAGTCACA |
| 951 | GAAAAGCATC TTACGGATGG CATGACAGTA AGAGAATTAT GCAGTGCTGC |
| 1001 | CATAACCATG AGTGATAACA CTGCGGCCAA CTTACTTCTG ACAACGATCG |
| 1051 | GAGGACCGAA GGAGCTAACC GCTTTTTTGC ACAACATGGG GGATCATGTA |
| 1101 | ACTCGCCTTG ATCGTTGGGA ACCGGAGCTG AATGAAGCCA TACCAAACGA |
| 1151 | CGAGCGTGAC ACCACGATGC CTGTAGCAAT GGCAACAACG TTGCGCAAAC |
| 1201 | TATTAACTGG CGAACTACTT ACTCTAGCTT CCCGGCAACA ATTAATAGAC |
| 1251 | TGGATGGAGG CGGATAAAGT TGCAGGACCA CTTCTGCGCT CGGCCCTTCC |
| 1301 | GGCTGGCTGG TTTATTGCTG ATAAATCTGG AGCCGGTGAG CGTGGGTCTC |
| 1351 | GCGGTATCAT TGCAGCACTG GGGCCAGATG GTAAGCCCTC CCGTATCGTA |
| 1401 | GTTATCTACA CGACGGGGAG TCAGGCAACT ATGGATGAAC GAAATAGACA |
| 1451 | GATCGCTGAG ATAGGTGCCT CACTGATTAA GCATTGGTAA CTGTCAGACC |
| 1501 | AAGTTTACTC ATATATACTT TAGATTGATT TAAAACTTCA TTTTTAATTT |
| 1551 | AAAAGGATCT AGGTGAAGAT CCTTTTTGAT AATCTCATGA CCAAAATCCC |
| 1601 | TTAACGTGAG TTTTCGTTCC ACTGAGCGTC AGACCCCGTA GAAAAGATCA |
| 1651 | AAGGATCTTC TTGAGATCCT TTTTTTCTGC GCGTAATCTG CTGCTTGCAA |
| 1701 | ACAAAAAAAC CACCGCTACC AGCGGTGGTT TGTTTGCCGG ATCAAGAGCT |
| 1751 | ACCAACTCTT TTTCCGAAGG TAACTGGCTT CAGCAGAGCG CAGATACCAA |
| 1801 | ATACTGTTCT TCTAGTGTAG CCGTAGTTAG GCCACCACTT CAAGAACTCT |
| 1851 | GTAGCACCGC CTACATACCT CGCTCTGCTA ATCCTGTTAC CAGTGGCTGC |
| 1901 | TGCCAGTGGC GATAAGTCGT GTCTTACCGG GTTGGACTCA AGACGATAGT |
| 1951 | TACCGGATAA GGCGCAGCGG TCGGGCTGAA CGGGGGGTTC GTGCACACAG |
| 2001 | CCCAGCTTGG AGCGAACGAC CTACACCGAA CTGAGATACC TACAGCGTGA |
| 2051 | GCTATGAGAA AGCGCCACGC TTCCCGAAGG GAGAAAGGCG GACAGGTATC |
| 2101 | CGGTAAGCGG CAGGGTCGGA ACAGGAGAGC GCACGAGGGA GCTTCCAGGG |
| 2151 | GGAAACGCCT GGTATCTTTA TAGTCCTGTC GGGTTTCGCC ACCTCTGACT |
| 2201 | TGAGCGTCGA TTTTTGTGAT GCTCGTCAGG GGGGCGGAGC CTATGGAAAA |
| 2251 | ACGCCAGCAA CGCGGCCTTT TTACGGTTCC TGGCCTTTTG CTGGCCTTTT |
| 2301 | GCTCACATGT TCTTTCCTGC GTTATCCCCT GATTCTGTGG ATAACCGTAT |
| 2351 | TACCGCCTTT GAGTGAGCTG ATACCGCTCG CCGCAGCCGA ACGACCGAGC |
| 2401 | GCAGCGAGTC AGTGAGCGAG GAAGCGGAAG AGCGCCCAAT ACGCAAACCG |
| 2451 | CCTCTCCCCG CGCGTTGGCC GATTCATTAA TGCAGCTGGC ACGACAGGTT |
| 2501 | TCCCGACTGG AAAGCGGGCA GTGAGCGCAA CGCAATTAAT GTGAGTTAGC |
| 2551 | TCACTCATTA GGCACCCCAG GCTTTACACT TTATGCTTCC GGCTCGTATG |
| 2601 | TTGTGTGGAA TTGTGAGCGG ATAACAATTT CACACAGGAA ACAGCTATGA |
| 2651 | CCATGATTAC GCCAAGCGCG CAATTAACCC TCACTAAAGG GAACAAAAGC |
| 2701 | TGGAGCTGCA AGCTTAATGT AGTCTTATGC AATACTCTTG TAGTCTTGCA |
| 2751 | ACATGGTAAC GATGAGTTAG CAACATGCCT TACAAGGAGA GAAAAAGCAC |
| 2801 | CGTGCATGCC GATTGGTGGA AGTAAGGTGG TACGATCGTG CCTTATTAGG |
| 2851 | AAGGCAACAG ACGGGTCTGA CATGGATTGG ACGAACCACT GAATTGCCGC |
| 2901 | ATTGCAGAGA TATTGTATTT AAGTGCCTAG CTCGATACAT AAACGGGTCT |
| 2951 | CTCTGGTTAG ACCAGATCTG AGCCTGGGAG CTCTCTGGCT AACTAGGGAA |
| 3001 | CCCACTGCTT AAGCCTCAAT AAAGCTTGCC TTGAGTGCTT CAAGTAGTGT |
| 3051 | GTGCCCGTCT GTTGTGTGAC TCTGGTAACT AGAGATCCCT CAGACCCTTT |
| 3101 | TAGTCAGTGT GGAAAATCTC TAGCAGTGGC GCCCGAACAG GGACTTGAAA |
| 3151 | GCGAAAGGGA AACCAGAGGA GCTCTCTCGA CGCAGGACTC GGCTTGCTGA |
| 3201 | AGCGCGCACG GCAAGAGGCG AGGGGCGGCG ACTGGTGAGT ACGCCAAAAA |
| 3251 | TTTTGACTAG CGGAGGCTAG AAGGAGAGAG ATGGGTGCGA GAGCGTCAGT |
| 3301 | ATTAAGCGGG GGAGAATTAG ATCGCGATGG GAAAAAATTC GGTTAAGGCC |
| 3351 | AGGGGGAAAG AAAAAATATA AATTAAAACA TATAGTATGG GCAAGCAGGG |
| 3401 | AGCTAGAACG ATTCGCAGTT AATCCTGGCC TGTTAGAAAC ATCAGAAGGC |
| 3451 | TGTAGACAAA TACTGGGACA GCTACAACCA TCCCTTCAGA CAGGATCAGA |
| 3501 | AGAACTTAGA TCATTATATA ATACAGTAGC AACCCTCTAT TGTGTGCATC |
| 3551 | AAAGGATAGA GATAAAAGAC ACCAAGGAAG CTTTAGACAA GATAGAGGAA |
| 3601 | GAGCAAAACA AAAGTAAGAC CACCGCACAG CAAGCGGCCG CTGATCTTCA |
| 3651 | GACCTGGAGG AGGAGATATG AGGGACAATT GGAGAAGTGA ATTATATAAA |
| 3701 | TATAAAGTAG TAAAAATTGA ACCATTAGGA GTAGCACCCA CCAAGGCAAA |
| 3751 | GAGAAGAGTG GTGCAGAGAG AAAAAAGAGC AGTGGGAATA GGAGCTTTGT |
| 3801 | TCCTTGGGTT CTTGGGAGCA GCAGGAAGCA CTATGGGCGC AGCGTCAATG |
| 3851 | ACGCTGACGG TACAGGCCAG ACAATTATTG TCTGGTATAG TGCAGCAGCA |
| 3901 | GAACAATTTG CTGAGGGCTA TTGAGGCGCA ACAGCATCTG TTGCAACTCA |
| 3951 | CAGTCTGGGG CATCAAGCAG CTCCAGGCAA GAATCCTGGC TGTGGAAAGA |
| 4001 | TACCTAAAGG ATCAACAGCT CCTGGGGATT TGGGGTTGCT CTGGAAAACT |
| 4051 | CATTTGCACC ACTGCTGTGC CTTGGAATGC TAGTTGGAGT AATAAATCTC |
| 4101 | TGGAACAGAT TTGGAATCAC ACGACCTGGA TGGAGTGGGA CAGAGAAATT |
| 4151 | AACAATTACA CAAGCTTAAT ACACTCCTTA ATTGAAGAAT CGCAAAACCA |
| 4201 | GCAAGAAAAG AATGAACAAG AATTATTGGA ATTAGATAAA TGGGCAAGTT |
| 4251 | TGTGGAATTG GTTTAACATA ACAAATTGGC TGTGGTATAT AAAATTATTC |
| 4301 | ATAATGATAG TAGGAGGCTT GGTAGGTTTA AGAATAGTTT TTGCTGTACT |
| 4351 | TTCTATAGTG AATAGAGTTA GGCAGGGATA TTCACCATTA TCGTTTCAGA |
| 4401 | CCCACCTCCC AACCCCGAGG GGACCCGGTA CCGAGGGCCT ATTTCCCATG |
| 4451 | ATTCCTTCAT ATTTGCATAT ACGATACAAG GCTGTTAGAG AGATAATTAG |
| 4501 | AATTAATTTG ACTGTAAACA CAAAGATATT AGTACAAAAT ACGTGACGTA |
| 4551 | GAAAGTAATA ATTTCTTGGG TAGTTTGCAG TTTTAAAATT ATGTTTTAAA |
| 4601 | ATGGACTATC ATATGCTTAC CGTAACTTGA AAGTATTTCG ATTTCTTGGC |
| 4651 | TTTATATATC TTGTGGAAAG GACGAAACAC CGGAGACGCT TTTTTCGTCT |
| 4701 | CAGTTTGAGA GCTAGAAATA GCAAGTTCAA ATAAGGCTAG TCCGTTATCA |
| 4751 | ACTTGAAAAA GTGGCACCGA GTCGGTGCTT TTTTGAATTC AAGCTTGGCG |
| 4801 | TAACTAGATC TTGAGACAAA TGGCAGTATT CATCCACAAT TTTAAAAGAA |
| 4851 | AAGGGGGGAT TGGGGGGTAC AGTGCAGGGG AAAGAATAGT AGACATAATA |
| 4901 | GCAACAGACA TACAAACTAA AGAATTACAA AAACAAATTA CAAAAATTCA |
| 4951 | AAATTTTCGG GTTTATTACA GGGACAGCAG AGATCCACTT TGGCGCCGGC |
| 5001 | TCGAGGGGGC CCGGGATAAC TTCGTATAGT ACACATTATA CGAAGTTATT |
| 5051 | GCAAAGATGG ATAAAGTTTT AAACAGAGAG GAATCTTTGC AGCTAATGGA |
| 5101 | CCTTCTAGGT CTTGAAAGGA GTGGGAATTG GCTCCGGTGC CCGTCAGTGG |
| 5151 | GCAGAGCGCA CATCGCCCAC AGTCCCCGAG AAGTTGGGGG GAGGGGTCGG |
| 5201 | CAATTGATCC GGTGCCTAGA GAAGGTGGCG CGGGGTAAAC TGGGAAAGTG |
| 5251 | ATGTCGTGTA CTGGCTCCGC CTTTTTCCCG AGGGTGGGGG AGAACCGTAT |
| 5301 | ATAAGTGCAG TAGTCGCCGT GAACGTTCTT TTTCGCAACG GGTTTGCCGC |
| 5351 | CAGAACACAG GTAAGTGCCG TGTGTGGTTC CCGCGGGCCT GGCCTCTTTA |
| 5401 | CGGGTTATGG CCCTTGCGTG CCTTGAATTA CTTCCACCTG GCTGCAGTAC |
| 5451 | GTGATTCTTG ATCCCGAGCT TCGGGTTGGA AGTGGGTGGG AGAGTTCGAG |
| 5501 | GCCTTGCGCT TAAGGAGCCC CTTCGCCTCG TGCTTGAGTT GAGGCCTGGC |
| 5551 | CTGGGCGCTG GGGCCGCCGC GTGCGAATCT GGTGGCACCT TCGCGCCTGT |
| 5601 | CTCGCTGCTT TCGATAAGTC TCTAGCCATT TAAAATTTTT GATGACCTGC |
| 5651 | TGCGACGCTT TTTTTCTGGC AAGATAGTCT TGTAAATGCG GGCCAAGATC |
| 5701 | TGCACACTGG TATTTCGGTT TTTGGGGCCG CGGGCGGCGA CGGGGCCCGT |
| 5751 | GCGTCCCAGC GCACATGTTC GGCGAGGCGG GGCCTGCGAG CGCGGCCACC |
| 5801 | GAGAATCGGA CGGGGGTAGT CTCAAGCTGG CCGGCCTGCT CTGGTGCCTG |
| 5851 | GCCTCGCGCC GCCGTGTATC GCCCCGCCCT GGGCGGCAAG GCTGGCCCGG |
| 5901 | TCGGCACCAG TTGCGTGAGC GGAAAGATGG CCGCTTCCCG GCCCTGCTGC |
| 5951 | AGGGAGCTCA AAATGGAGGA CGCGGCGCTC GGGAGAGCGG GCGGGTGAGT |
| 6001 | CACCCACACA AAGGAAAAGG GCCTTTCCGT CCTCAGCCGT CGCTTCATGT |
| 6051 | GACTCCACGG AGTACCGGGC GCCGTCCAGG CACCTCGATT AGTTCTCGAG |
| 6101 | CTTTTGGAGT ACGTCGTCTT TAGGTTGGGG GGAGGGGTTT TATGCGATGG |
| 6151 | AGTTTCCCCA CACTGAGTGG GTGGAGACTG AAGTTAGGCC AGCTTGGCAC |
| 6201 | TTGATGTAAT TCTCCTTGGA ATTTGCCCTT TTTGAGTTTG GATCTTGGTT |
| 6251 | CATTCTCAAG CCTCAGACAG TGGTTCAAAG TTTTTTTCTT CCATTTCAGG |
| 6301 | TGTCGTGACG TACGGCCACC ATGACCGAGT ACAAGCCCAC GGTGCGCCTC |
| 6351 | GCCACCCGCG ACGACGTCCC CAGGGCCGTA CGCACCCTCG CCGCCGCGTT |
| 6401 | CGCCGACTAC CCCGCCACGC GCCACACCGT CGATCCGGAC CGCCACATCG |
| 6451 | AGCGGGTCAC CGAGCTGCAA GAACTCTTCC TCACGCGCGT CGGGCTCGAC |
| 6501 | ATCGGCAAGG TGTGGGTCGC GGACGACGGC GCCGCCGTGG CGGTCTGGAC |
| 6551 | CACGCCGGAG AGCGTCGAAG CGGGGGCGGT GTTCGCCGAG ATCGGCCCGC |
| 6601 | GCATGGCCGA GTTGAGCGGT TCCCGGCTGG CCGCGCAGCA ACAGATGGAA |
| 6651 | GGCCTCCTGG CGCCGCACCG GCCCAAGGAG CCCGCGTGGT TCCTGGCCAC |
| 6701 | CGTCGGCGTT TCGCCCGACC ACCAGGGCAA GGGTCTGGGC AGCGCCGTCG |
| 6751 | TGCTCCCCGG AGTGGAGGCG GCCGAGCGCG CCGGGGTGCC CGCCTTCCTG |
| 6801 | GAGACCTCCG CGCCCCGCAA CCTCCCCTTC TACGAGCGGC TCGGCTTCAC |
| 6851 | CGTCACCGCC GACGTCGAGG TGCCCGAAGG ACCGCGCACC TGGTGCATGA |
| 6901 | CCCGCAAGCC CGGTGCCGCT AGCCTGCAGG GATCCGGCGC AACAAACTTC |
| 6951 | TCTCTGCTGA AACAAGCCGG AGATGTCGAA GAGAATCCTG GACCGGCTAG |
| 7001 | CATGGTGAGC GAGCTGATTA AGGAGAACAT GCACATGAAG CTGTACATGG |
| 7051 | AGGGCACCGT GAACAACCAC CACTTCAAGT GCACATCCGA GGGCGAAGGC |
| 7101 | AAGCCCTACG AGGGCACCCA GACCATGAGA ATCAAGGCGG TCGAGGGCGG |
| 7151 | CCCTCTCCCC TTCGCCTTCG ACATCCTGGC TACCAGCTTC ATGTACGGCA |
| 7201 | GCAAAACCTT CATCAACCAC ACCCAGGGCA TCCCCGACTT CTTTAAGCAG |
| 7251 | TCCTTCCCCG AGGGCTTCAC ATGGGAGAGA GTCACCACAT ACGAAGACGG |
| 7301 | GGGCGTGCTG ACCGCTACCC AGGACACCAG CCTCCAGGAC GGCTGCCTCA |
| 7351 | TCTACAACGT CAAGATCAGA GGGGTGAACT TCCCATCCAA CGGCCCTGTG |
| 7401 | ATGCAGAAGA AAACACTCGG CTGGGAGGCC TCCACCGAGA CCCTGTACCC |
| 7451 | CGCTGACGGC GGCCTGGAAG GCAGAGCCGA CATGGCCCTG AAGCTCGTGG |
| 7501 | GCGGGGGCCA CCTGATCTGC AACTTGAAGA CCACATACAG ATCCAAGAAA |
| 7551 | CCCGCTAAGA ACCTCAAGAT GCCCGGCGTC TACTATGTGG ACAGAAGACT |
| 7601 | GGAAAGAATC AAGGAGGCCG ACAAAGAGAC CTACGTCGAG CAGCACGAGG |
| 7651 | TGGCTGTGGC CAGATACTGC GACCTCCCTA GCAAACTGGG GCACAGATAA |
| 7701 | ATAACTTCGT ATAGTACACA TTATACGAAG TTATACGCGT TAAGTCGACA |
| 7751 | ATCAACCTCT GGATTACAAA ATTTGTGAAA GATTGACTGG TATTCTTAAC |
| 7801 | TATGTTGCTC CTTTTACGCT ATGTGGATAC GCTGCTTTAA TGCCTTTGTA |
| 7851 | TCATGCTATT GCTTCCCGTA TGGCTTTCAT TTTCTCCTCC TTGTATAAAT |
| 7901 | CCTGGTTGCT GTCTCTTTAT GAGGAGTTGT GGCCCGTTGT CAGGCAACGT |
| 7951 | GGCGTGGTGT GCACTGTGTT TGCTGACGCA ACCCCCACTG GTTGGGGCAT |
| 8001 | TGCCACCACC TGTCAGCTCC TTTCCGGGAC TTTCGCTTTC CCCCTCCCTA |
| 8051 | TTGCCACGGC GGAACTCATC GCCGCCTGCC TTGCCCGCTG CTGGACAGGG |
| 8101 | GCTCGGCTGT TGGGCACTGA CAATTCCGTG GTGTTGTCGG GGAAATCATC |
| 8151 | GTCCTTTCCT TGGCTGCTCG CCTGTGTTGC CACCTGGATT CTGCGCGGGA |
| 8201 | CGTCCTTCTG CTACGTCCCT TCGGCCCTCA ATCCAGCGGA CCTTCCTTCC |
| 8251 | CGCGGCCTGC TGCCGGCTCT GCGGCCTCTT CCGCGTCTTC GCCTTCGCCC |
| 8301 | TCAGACGAGT CGGATCTCCC TTTGGGCCGC CTCCCCGCGT CGACTTTAAG |
| 8351 | ACCAATGACT TACAAGGCAG CTGTAGATCT TAGCCACTTT TTAAAAGAAA |
| 8401 | AGGGGGGACT GGAAGGGCTA ATTCACTCCC AACGAAGACA AGATCTGCTT |
| 8451 | TTTGCTTGTA CTGGGTCTCT CTGGTTAGAC CAGATCTGAG CCTGGGAGCT |
| 8501 | CTCTGGCTAA CTAGGGAACC CACTGCTTAA GCCTCAATAA AGCTTGCCTT |
| 8551 | GAGTGCTTCA AGTAGTGTGT GCCCGTCTGT TGTGTGACTC TGGTAACTAG |
| 8601 | AGATCCCTCA GACCCTTTTA GTCAGTGTGG AAAATCTCTA GCAGTACGTA |
| 8651 | TAGTAGTTCA TGTCATCTTA TTATTCAGTA TTTATAACTT GCAAAGAAAT |
| 8701 | GAATATCAGA GAGTGAGAGG AACTTGTTTA TTGCAGCTTA TAATGGTTAC |
| 8751 | AAATAAAGCA ATAGCATCAC AAATTTCACA AATAAAGCAT TTTTTTCACT |
| 8801 | GCATTCTAGT TGTGGTTTGT CCAAACTCAT CAATGTATCT TATCATGTCT |
| 8851 | GGCTCTAGCT ATCCCGCCCC TAACTCCGCC CATCCCGCCC CTAACTCCGC |
| 8901 | CCAGTTCCGC CCATTCTCCG CCCCATGGCT GACTAATTTT TTTTATTTAT |
| 8951 | GCAGAGGCCG AGGCCGCCTC GGCCTCTGAG CTATTCCAGA AGTAGTGAGG |
| 9001 | AGGCTTTTTT GGAGGCCTAG GGACGTACCC AATTCGCCCT ATAGTGAGTC |
| 9051 | GTATTACGCG CGCTCACTGG CCGTCGTTTT ACAACGTCGT GACTGGGAAA |
| 9101 | ACCCTGGCGT TACCCAACTT AATCGCCTTG CAGCACATCC CCCTTTCGCC |
| 9151 | AGCTGGCGTA ATAGCGAAGA GGCCCGCACC |

### ANNOTATIONS

44-499: F1 ori
630-1490: AmpR
1638-2305: pUC ori
2714-3126: 5′ LTR
3177-3314: psi
3281-3645: gag
3791-4032: Rev response element (RRE)
4433-4673: U6 (promoter)
4703-4778: sgRNA scaffold
4840-4957: cPPT/CTS
5016-5049: lox5171
5050-6308: EF1α (promoter)
6321-6923: PuroR
6930-6995: P2A
7002-7700: mKate
7701-7734: lox5171
7750-8338: WPRE
8409-8644: 3′ LTR (SIN)
8721-8851: SV40 polyadenylation signal
8932-9006: SV40 origin of replication

| EFS_Cre: | |
|---|---|
| (SEQ ID NO: 10) | |
| 1 | ACCGGTTAAG TCGACAATCA ACGCGTTAAG TCGACAATCA ACCTCTGGAT |
| 51 | TACAAAATTT GTGAAAGATT GACTGGTATT CTTAACTATG TTGCTCCTTT |
| 101 | TACGCTATGT GGATACGCTG CTTTAATGCC TTTGTATCAT GCTATTGCTT |
| 151 | CCCGTATGGC TTTCATTTTC TCCTCCTTGT ATAAATCCTG GTTGCTGTCT |
| 201 | CTTTATGAGG AGTTGTGGCC CGTTGTCAGG CAACGTGGCG TGGTGTGCAC |
| 251 | TGTGTTTGCT GACGCAACCC CCACTGGTTG GGGCATTGCC ACCACCTGTC |
| 301 | AGCTCCTTTC CGGGACTTTC GCTTTCCCCC TCCCTATTGC CACGGCGGAA |
| 351 | CTCATCGCCG CCTGCCTTGC CCGCTGCTGG ACAGGGGCTC GGCTGTTGGG |
| 401 | CACTGACAAT TCCGTGGTGT TGTCGGGGAA ATCATCGTCC TTTCCTTGGC |
| 451 | TGCTCGCCTG TGTTGCCACC TGGATTCTGC GCGGGACGTC CTTCTGCTAC |
| 501 | GTCCCTTCGG CCCTCAATCC AGCGGACCTT CCTTCCCGCG GCCTGCTGCC |
| 551 | GGCTCTGCGG CCTCTTCCGC GTCTTCGCCT TCGCCCTCAG ACGAGTCGGA |
| 601 | TCTCCCTTTG GGCCGCCTCC CCGCGTCGAC TTTAAGACCA ATGACTTACA |
| 651 | AGGCAGCTGT AGATCTTAGC CACTTTTTAA AAGAAAAGGG GGGACTGGAA |
| 701 | GGGCTAATTC ACTCCCAACG AAGACAAGAT CTGCTTTTTG CTTGTACTGG |
| 751 | GTCTCTCTGG TTAGACCAGA TCTGAGCCTG GGAGCTCTCT GGCTAACTAG |
| 801 | GGAACCCACT GCTTAAGCCT CAATAAAGCT TGCCTTGAGT GCTTCAAGTA |
| 851 | GTGTGTGCCC GTCTGTTGTG TGACTCTGGT AACTAGAGAT CCCTCAGACC |
| 901 | CTTTTAGTCA GTGTGGAAAA TCTCTAGCAG TACGTATAGT AGTTCATGTC |
| 951 | ATCTTATTAT TCAGTATTTA TAACTTGCAA AGAAATGAAT ATCAGAGAGT |
| 1001 | GAGAGGAACT TGTTTATTGC AGCTTATAAT GGTTACAAAT AAAGCAATAG |
| 1051 | CATCACAAAT TTCACAAATA AAGCATTTTT TTCACTGCAT TCTAGTTGTG |
| 1101 | GTTTGTCCAA ACTCATCAAT GTATCTTATC ATGTCTGGCT CTAGCTATCC |
| 1151 | CGCCCCTAAC TCCGCCCATC CCGCCCCTAA CTCCGCCCAG TTCCGCCCAT |
| 1201 | TCTCCGCCCC ATGGCTGACT AATTTTTTTT ATTTATGCAG AGGCCGAGGC |
| 1251 | CGCCTCGGCC TCTGAGCTAT TCCAGAAGTA GTGAGGAGGC TTTTTTGGAG |
| 1301 | GCCTAGGGAC GTACCCAATT CGCCCTATAG TGAGTCGTAT TACGCGCGCT |
| 1351 | CACTGGCCGT CGTTTTACAA CGTCGTGACT GGGAAAACCC TGGCGTTACC |
| 1401 | CAACTTAATC GCCTTGCAGC ACATCCCCCT TTCGCCAGCT GGCGTAATAG |
| 1451 | CGAAGAGGCC CGCACCGATC GCCCTTCCCA ACAGTTGCGC AGCCTGAATG |
| 1501 | GCGAATGGGA CGCGCCCTGT AGCGGCGCAT TAAGCGCGGC GGGTGTGGTG |
| 1551 | GTTACGCGCA GCGTGACCGC TACACTTGCC AGCGCCCTAG CGCCCGCTCC |
| 1601 | TTTCGCTTTC TTCCCTTCCT TTCTCGCCAC GTTCGCCGGC TTTCCCCGTC |
| 1651 | AAGCTCTAAA TCGGGGGCTC CCTTTAGGGT TCCGATTTAG TGCTTTACGG |
| 1701 | CACCTCGACC CCAAAAAACT TGATTAGGGT GATGGTTCAC GTAGTGGGCC |
| 1751 | ATCGCCCTGA TAGACGGTTT TTCGCCCTTT GACGTTGGAG TCCACGTTCT |
| 1801 | TTAATAGTGG ACTCTTGTTC CAAACTGGAA CAACACTCAA CCCTATCTCG |
| 1851 | GTCTATTCTT TTGATTTATA AGGGATTTTG CCGATTTCGG CCTATTGGTT |
| 1901 | AAAAAATGAG CTGATTTAAC AAAAATTTAA CGCGAATTTT AACAAAATAT |
| 1951 | TAACGCTTAC AATTTAGGTG GCACTTTTCG GGGAAATGTG CGCGGAACCC |
| 2001 | CTATTTGTTT ATTTTTCTAA ATACATTCAA ATATGTATCC GCTCATGAGA |
| 2051 | CAATAACCCT GATAAATGCT TCAATAATAT TGAAAAAGGA AGAGTATGAG |
| 2101 | TATTCAACAT TTCCGTGTCG CCCTTATTCC CTTTTTTGCG GCATTTTGCC |
| 2151 | TTCCTGTTTT TGCTCACCCA GAAACGCTGG TGAAAGTAAA AGATGCTGAA |
| 2201 | GATCAGTTGG GTGCACGAGT GGGTTACATC GAACTGGATC TCAACAGCGG |
| 2251 | TAAGATCCTT GAGAGTTTTC GCCCCGAAGA ACGTTTTCCA ATGATGAGCA |
| 2301 | CTTTTAAAGT TCTGCTATGT GGCGCGGTAT TATCCCGTAT TGACGCCGGG |
| 2351 | CAAGAGCAAC TCGGTCGCCG CATACACTAT TCTCAGAATG ACTTGGTTGA |
| 2401 | GTACTCACCA GTCACAGAAA AGCATCTTAC GGATGGCATG ACAGTAAGAG |
| 2451 | AATTATGCAG TGCTGCCATA ACCATGAGTG ATAACACTGC GGCCAACTTA |
| 2501 | CTTCTGACAA CGATCGGAGG ACCGAAGGAG CTAACCGCTT TTTTGCACAA |
| 2551 | CATGGGGGAT CATGTAACTC GCCTTGATCG TTGGGAACCG GAGCTGAATG |
| 2601 | AAGCCATACC AAACGACGAG CGTGACACCA CGATGCCTGT AGCAATGGCA |
| 2651 | ACAACGTTGC GCAAACTATT AACTGGCGAA CTACTTACTC TAGCTTCCCG |
| 2701 | GCAACAATTA ATAGACTGGA TGGAGGCGGA TAAAGTTGCA GGACCACTTC |
| 2751 | TGCGCTCGGC CCTTCCGGCT GGCTGGTTTA TTGCTGATAA ATCTGGAGCC |
| 2801 | GGTGAGCGTG GGTCTCGCGG TATCATTGCA GCACTGGGGC CAGATGGTAA |
| 2851 | GCCCTCCCGT ATCGTAGTTA TCTACACGAC GGGGAGTCAG GCAACTATGG |
| 2901 | ATGAACGAAA TAGACAGATC GCTGAGATAG GTGCCTCACT GATTAAGCAT |
| 2951 | TGGTAACTGT CAGACCAAGT TTACTCATAT ATACTTTAGA TTGATTTAAA |
| 3001 | ACTTCATTTT TAATTTAAAA GGATCTAGGT GAAGATCCTT TTTGATAATC |
| 3051 | TCATGACCAA AATCCCTTAA CGTGAGTTTT CGTTCCACTG AGCGTCAGAC |
| 3101 | CCCGTAGAAA AGATCAAAGG ATCTTCTTGA GATCCTTTTT TTCTGCGCGT |
| 3151 | AATCTGCTGC TTGCAAACAA AAAAACCACC GCTACCAGCG GTGGTTTGTT |
| 3201 | TGCCGGATCA AGAGCTACCA ACTCTTTTTC CGAAGGTAAC TGGCTTCAGC |
| 3251 | AGAGCGCAGA TACCAAATAC TGTTCTTCTA GTGTAGCCGT AGTTAGGCCA |
| 3301 | CCACTTCAAG AACTCTGTAG CACCGCCTAC ATACCTCGCT CTGCTAATCC |
| 3351 | TGTTACCAGT GGCTGCTGCC AGTGGCGATA AGTCGTGTCT TACCGGGTTG |
| 3401 | GACTCAAGAC GATAGTTACC GGATAAGGCG CAGCGGTCGG GCTGAACGGG |
| 3451 | GGGTTCGTGC ACACAGCCCA GCTTGGAGCG AACGACCTAC ACCGAACTGA |
| 3501 | GATACCTACA GCGTGAGCTA TGAGAAAGCG CCACGCTTCC CGAAGGGAGA |
| 3551 | AAGGCGGACA GGTATCCGGT AAGCGGCAGG GTCGGAACAG GAGAGCGCAC |
| 3601 | GAGGGAGCTT CCAGGGGGAA ACGCCTGGTA TCTTTATAGT CCTGTCGGGT |
| 3651 | TTCGCCACCT CTGACTTGAG CGTCGATTTT TGTGATGCTC GTCAGGGGGG |
| 3701 | CGGAGCCTAT GGAAAAACGC CAGCAACGCG GCCTTTTTAC GGTTCCTGGC |
| 3751 | CTTTTGCTGG CCTTTTGCTC ACATGTTCTT TCCTGCGTTA TCCCCTGATT |
| 3801 | CTGTGGATAA CCGTATTACC GCCTTTGAGT GAGCTGATAC CGCTCGCCGC |
| 3851 | AGCCGAACGA CCGAGCGCAG CGAGTCAGTG AGCGAGGAAG CGGAAGAGCG |
| 3901 | CCCAATACGC AAACCGCCTC TCCCCGCGCG TTGGCCGATT CATTAATGCA |
| 3951 | GCTGGCACGA CAGGTTTCCC GACTGGAAAG CGGGCAGTGA GCGCAACGCA |
| 4001 | ATTAATGTGA GTTAGCTCAC TCATTAGGCA CCCCAGGCTT TACACTTTAT |
| 4051 | GCTTCCGGCT CGTATGTTGT GTGGAATTGT GAGCGGATAA CAATTTCACA |
| 4101 | CAGGAAACAG CTATGACCAT GATTACGCCA AGCGCGCAAT TAACCCTCAC |
| 4151 | TAAAGGGAAC AAAAGCTGGA GCTGCAAGCT TAATGTAGTC TTATGCAATA |
| 4201 | CTCTTGTAGT CTTGCAACAT GGTAACGATG AGTTAGCAAC ATGCCTTACA |
| 4251 | AGGAGAGAAA AAGCACCGTG CATGCCGATT GGTGGAAGTA AGGTGGTACG |
| 4301 | ATCGTGCCTT ATTAGGAAGG CAACAGACGG GTCTGACATG GATTGGACGA |
| 4351 | ACCACTGAAT TGCCGCATTG CAGAGATATT GTATTTAAGT GCCTAGCTCG |
| 4401 | ATACATAAAC GGGTCTCTCT GGTTAGACCA GATCTGAGCC TGGGAGCTCT |
| 4451 | CTGGCTAACT AGGGAACCCA CTGCTTAAGC CTCAATAAAG CTTGCCTTGA |
| 4501 | GTGCTTCAAG TAGTGTGTGC CCGTCTGTTG TGTGACTCTG GTAACTAGAG |
| 4551 | ATCCCTCAGA CCCTTTTAGT CAGTGTGGAA AATCTCTAGC AGTGGCGCCC |
| 4601 | GAACAGGGAC TTGAAAGCGA AAGGGAAACC AGAGGAGCTC TCTCGACGCA |
| 4651 | GGACTCGGCT TGCTGAAGCG CGCACGGCAA GAGGCGAGGG GCGGCGACTG |
| 4701 | GTGAGTACGC CAAAAATTTT GACTAGCGGA GGCTAGAAGG AGAGAGATGG |
| 4751 | GTGCGAGAGC GTCAGTATTA AGCGGGGGAG AATTAGATCG CGATGGGAAA |
| 4801 | AAATTCGGTT AAGGCCAGGG GGAAAGAAAA AATATAAATT AAAACATATA |
| 4851 | GTATGGGCAA GCAGGGAGCT AGAACGATTC GCAGTTAATC CTGGCCTGTT |
| 4901 | AGAAACATCA GAAGGCTGTA GACAAATACT GGGACAGCTA CAACCATCCC |
| 4951 | TTCAGACAGG ATCAGAAGAA CTTAGATCAT TATATAATAC AGTAGCAACC |
| 5001 | CTCTATTGTG TGCATCAAAG GATAGAGATA AAAGACACCA AGGAAGCTTT |
| 5051 | AGACAAGATA GAGGAAGAGC AAAACAAAAG TAAGACCACC GCACAGCAAG |
| 5101 | CGGCCGCTGA TCTTCAGACC TGGAGGAGGA GATATGAGGG ACAATTGGAG |
| 5151 | AAGTGAATTA TATAAATATA AAGTAGTAAA AATTGAACCA TTAGGAGTAG |
| 5201 | CACCCACCAA GGCAAAGAGA AGAGTGGTGC AGAGAGAAAA AAGAGCAGTG |
| 5251 | GGAATAGGAG CTTTGTTCCT TGGGTTCTTG GGAGCAGCAG GAAGCACTAT |
| 5301 | GGGCGCAGCG TCAATGACGC TGACGGTACA GGCCAGACAA TTATTGTCTG |
| 5351 | GTATAGTGCA GCAGCAGAAC AATTTGCTGA GGGCTATTGA GGCGCAACAG |
| 5401 | CATCTGTTGC AACTCACAGT CTGGGGCATC AAGCAGCTCC AGGCAAGAAT |
| 5451 | CCTGGCTGTG GAAAGATACC TAAAGGATCA ACAGCTCCTG GGGATTTGGG |
| 5501 | GTTGCTCTGG AAAACTCATT TGCACCACTG CTGTGCCTTG GAATGCTAGT |
| 5551 | TGGAGTAATA AATCTCTGGA ACAGATTTGG AATCACACGA CCTGGATGGA |
| 5601 | GTGGGACAGA GAAATTAACA ATTACACAAG CTTAATACAC TCCTTAATTG |
| 5651 | AAGAATCGCA AAACCAGCAA GAAAAGAATG AACAAGAATT ATTGGAATTA |
| 5701 | GATAAATGGG CAAGTTTGTG GAATTGGTTT AACATAACAA ATTGGCTGTG |
| 5751 | GTATATAAAA TTATTCATAA TGATAGTAGG AGGCTTGGTA GGTTTAAGAA |
| 5801 | TAGTTTTTGC TGTACTTTCT ATAGTGAATA GAGTTAGGCA GGGATATTCA |
| 5851 | CCATTATCGT TTCAGACCCA CCTCCCAACC CCGAGGGGAC CCATGCATCC |
| 5901 | ACAATTTTAA AAGAAAAGGG GGGATTGGGG GGTACAGTGC AGGGGAAAGA |
| 5951 | ATAGTAGACA TAATAGCAAC AGACATACAA ACTAAAGAAT TACAAAAACA |
| 6001 | AATTACAAAA ATTCAAAATT TTCGGGTTTA TTACAGGGAC AGCAGAGATC |
| 6051 | CAGTTTGGTT AATTAAGCTA GCTAGGTCTT GAAAGGAGTG GGAATTGGCT |
| 6101 | CCGGTGCCCG TCAGTGGGCA GAGCGCACAT CGCCCACAGT CCCCGAGAAG |
| 6151 | TTGGGGGGAG GGGTCGGCAA TTGATCCGGT GCCTAGAGAA GGTGGCGCGG |
| 6201 | GGTAAACTGG GAAAGTGATG TCGTGTACTG GCTCCGCCTT TTTCCCGAGG |
| 6251 | GTGGGGGAGA ACCGTATATA AGTGCAGTAG TCGCCGTGAA CGTTCTTTTT |
| 6301 | CGCAACGGGT TTGCCGCCAG AACACAGGAC CGGTTCTAGA GCGCTGCCAC |
| 6351 | CATGGCTAAT CTCCTGACCG TGCATCAGAA TCTGCCTGCC CTGCCCGTCG |
| 6401 | ACGCAACAAG CGATGAAGTC CGCAAGAATC TCATGGACAT GTTCAGGGAC |
| 6451 | AGACAGGCCT TTTCCGAGCA CACCTGGAAG ATGCTGCTGA GCGTGTGCAG |
| 6501 | GTCCTGGGCT GCTTGGTGTA AGCTGAACAA CAGAAAGTGG TTCCCAGCTG |
| 6551 | AGCCAGAGGA CGTGCGGGAT TACCTGCTGT ACCTGCAGGC CCGCGGACTG |
| 6601 | GCTGTGAAGA CAATCCAGCA GCACCTGGGC CAGCTGAACA TGCTGCACAG |
| 6651 | GAGAAGCGGA CTGCCCCGGC CTAGCGACTC CAACGCCGTG AGCCTGGTCA |
| 6701 | TGCGGCGCAT CAGGAAGGAG AACGTGGATG CCGGCGAGAG AGCTAAGCAG |
| 6751 | GCCCTGGCTT TCGAGAGGAC CGACTTTGAT CAGGTGAGAT CTCTGATGGA |
| 6801 | GAACAGCGAC AGGTGCCAGG ATATCAGAAA CCTGGCCTTT CTGGGAATCG |
| 6851 | CTTACAACAC CCTGCTGAGA ATCGCCGAGA TCGCTCGGAT CCGCGTGAAG |
| 6901 | GACATCTCTC GGACAGATGG CGGACGCATG CTGATCCACA TCGGCAGGAC |
| 6951 | CAAGACACTG GTGTCCACCG CCGGCGTGGA GAAGGCTCTG TCTCTGGGAG |
| 7001 | TGACAAAGCT GGTGGAGAGA TGGATCTCTG TGAGCGGCGT GGCCGACGAT |
| 7051 | CCTAACAACT ACCTGTTCTG TAGGGTGAGA AAGAACGGAG TGGCCGCTCC |
| 7101 | ATCCGCTACC TCTCAGCTGA GCACACGGGC CCTGGAGGGC ATCTTTGAGG |
| 7151 | CTACCCACCG CCTGATCTAC GGCGCCAAGG ACGATTCTGG ACAGCGGTAC |
| 7201 | CTGGCTTGGT CCGGACACTC TGCTCGCGTG GGAGCTGCTC GGGATATGGC |
| 7251 | CCGCGCTGGC GTGAGCATCC CAGAGATCAT GCAGGCCGGC GGATGGACAA |
| 7301 | ACGTGAACAT CGTGATGAAC TACATTAGAA ATCTGGATAG CGAAACTGGG |
| 7351 | GCAATGGTGC GGCTGCTGGA GGATGGGGAC TGATAGTAAT GAACTAGT |

### ANNOTATIONS

36-624: WPRE
695-930: 3′ LTR (SIN)
1007-1137: SV40 polyadenylation signal
1217-1292: SV40 origin of replication
1510-1965: F1 ori
2096-2956: AmpR
3104-3771: pUC ori
4180-4592: 5′ LTR
4643-4780: psi
4747-5111: gag
5257-5498: Rev response element (RRE)
5905-6022: cPPT
6073-6328: EFS (promoter)
6352-7383: Cre

What is claimed is:

1. A method of producing a population of genetically modified cells, comprising:
(i) providing a population of cells;
(ii) introducing a first integration vector into at least a portion of the population of cells,
wherein the first integration vector is a replication defective retroviral vector derived from a primate lentivirus,
wherein the first integration vector comprises a first nucleic acid sequence comprising a first promoter operably linked to a Cas protein coding sequence encoding a Cas protein; and at least a first 3′ site-specific recombination site located 3′ to the Cas coding sequence, and
wherein the first integrating vector is capable of integration into the genomes of at least a portion of the population of cells;
(iii) introducing an sgRNA into at least a portion of the population of cells, wherein the sgRNA is capable of guiding the Cas protein to a target site in the genomes of at least a portion of the population of cells, and wherein the Cas protein is capable of double-stranded DNA cleavage at the target site;
(iv) culturing the population of cells for a time sufficient for (a) integration of the first integrating vector into the genomes of at least a portion of the population of cells; and (b) induction of a genetic modification at the target site in the genomes of at least a portion of the population of cells by double-stranded DNA cleavage by the Cas protein and the sgRNA; and
(v) introducing a first recombinase into at least a portion of the population of cells, wherein the first recombinase catalyzes recombination between the first 3′ site-specific recombination site and a first 5′ site-specific recombination site located 5′ to at least the Cas protein coding sequence, thereby causing excision of the Cas protein coding sequence from the genomes of at least a portion of the population of cells.

2. The method of claim 1, wherein the first 3′ site-specific recombination site is located within a 3′ long terminal repeat (LTR) region at the 3′ end of the first integration vector and is duplicated during integration to produce the first 5′ site-specific recombination site located within a 5′ long terminal repeat (LTR) at the 5′ end of the first integration vector.

3. The method of claim 1, wherein the first integration vector further comprises a first 5′ site-specific recombination site located 5′ of at least the Cas protein coding sequence.

4. The method of any one of claims 1-3, wherein the Cas protein is a Cas9, a Cpf1, an SaCas9, or a Cas9 analog.

5. The method of any one of claims 1-4, wherein the first integrating vector further comprises a second coding sequence encoding a first detectable marker.

6. The method of claim 5, wherein the first coding sequence encoding the Cas protein is operably linked to the second coding sequence encoding the first detectable marker.

7. The method of any one of claims 1-6, wherein the first coding sequence encoding the Cas protein and the second coding sequence encoding the first detectable marker are linked by a first spacer.

8. The method of any one of claims 1-7, wherein the first detectable marker is an antibiotic resistance gene.

9. The method of claim 8, wherein the antibiotic resistance gene is a bls gene, hph gene, sh ble gene or geo gene.

10. The method of any one of claims 1-7, wherein the first detectable marker is a fluorescent protein gene.

11. The method of claim 10, wherein the fluorescent protein is GFP, RFP, tdtomato, mcherry, CFP, YFP, or BFP.

12. The method of any one of claims 1-7, wherein the first detectable marker is a cell surface marker.

13. The method of any one of claims 1-7, wherein the first detectable marker is luciferase or beta-galactosidase.

14. The method of claim 7, where in the first spacer is a third coding sequence encoding a peptide.

15. The method of claim 14, wherein the peptide comprises a cleavage site for a protease.

16. The method of claim 15, wherein the protease is an endogenous protease.

17. The method of any one of claims 14-16, wherein the peptide is a 2A peptide.

18. The method of claim 17, wherein the 2A peptide is a P2A peptide or a T2A peptide.

19. The method of claim 7, wherein the first spacer is an internal ribosome entry site (IRES).

20. The method of any one of claims 1-19, wherein the first promoter is a constitutive promoter, an inducible promoter or a tissue specific promoter.

21. The method of any one of claims 1-20, wherein the first integrating vector further comprises a transcription enhancer sequence.

22. The method of claim 21, wherein the transcription enhancer sequence is a woodchuck hepatitis virus post-transcriptional regulatory element (WPRE) sequence.

23. The method of any one of claims 1-22, wherein the first integrating vector is a lentiviral vector.

24. The method of any one of claims 1-23, wherein the first 5′ paired site-specific recombination site and the first 3′ paired site-specific recombination site flank at least the first coding sequence encoding the Cas protein and the second coding sequence encoding the first detectable marker.

25. The method of any one of claims 1-24, wherein the first 5′ paired site-specific recombination site and the first 3′ paired site-specific recombination site flank at least the first coding sequence encoding the Cas protein, the second coding sequence encoding the first detectable marker and the first promoter.

26. The method of any one of claims 21-25, wherein the first 5′ paired site-specific recombination site and the first 3′ paired site-specific recombination site flank at least the first coding sequence encoding the Cas protein, the second coding sequence encoding the first detectable marker, the first promoter and the enhancer sequence.

27. The method of any one of claims 1-25, wherein the first integrating vector further comprises a second promoter operably linked to a fourth coding sequence encoding a second detectable marker.

28. The method of claim 27, wherein the second detectable marker is an antibiotic resistance gene.

29. The method of claim 28, wherein the antibiotic resistant gene is a bls gene, hph gene, sh ble gene or geo gene.

30. The method of claim 27, wherein the second detectable marker is a fluorescent protein gene.

31. The method of any one of claim 30, wherein the fluorescent protein is a GFP, RFP, tdtomato, mcherry, CFP, YFP, or BFP gene.

32. The method of claim 27, wherein the second detectable marker is a cell surface marker.

33. The method of claim 27, wherein the second detectable marker is luciferase or beta-galactosidase.

34. The method of any one of claims 27-33, wherein the second promoter is a constitutive promoter, an inducible promoter or a tissue specific promoter.

35. The method of any one of claims 27-34, wherein the first detectable marker and the second detectable marker are different.

36. The method of any one of claims 27-35, wherein the first 5′ paired site-specific recombination site and the first 3′ paired site-specific recombination site flank at least the first coding sequence encoding the Cas protein and the second coding sequence encoding the first detectable marker.

37. The method of any one of claims 27-35, wherein the first 5′ paired site-specific recombination site and the first 3′ paired site-specific recombination site flank at least the first coding sequence encoding the Cas protein, the second coding sequence encoding the first detectable marker and the first promoter.

38. The method of any one of claims 27-35, wherein the first 5′ paired site-specific recombination site and the first 3′ paired site-specific recombination site flank at least the first coding sequence encoding the Cas protein, the second coding sequence encoding the first detectable marker, the first promoter and the fourth coding sequence encoding the second detectable marker.

39. The method of any one of claims 27-35, wherein the first 5′ paired site-specific recombination site and the first 3′ paired site-specific recombination site flank at least the first coding sequence encoding the Cas protein, the second coding sequence encoding the first detectable marker, the first promoter, the fourth coding sequence encoding the second detectable marker and the second promoter.

40. The method of any one of claims 27-35, wherein the first 5′ paired site-specific recombination site and the first 3′ paired site-specific recombination site flank at least the first coding sequence encoding the Cas protein, the second coding sequence encoding the first detectable marker, the first promoter, the fourth coding sequence encoding the second detectable marker, the second promoter and the enhancer sequence.

41. The method of any one of claims 1-40, wherein the sgRNA is delivered into at least a portion of the population of cells as a single strand RNA.

42. The method of any one of claims 1-40, wherein the sgRNA is delivered into at least a portion of the population of cells by the first integrating vector.

43. The method of claim 42, wherein the first integrating vector further comprises a U6 promoter operably linked to a fifth coding sequence encoding the sgRNA.

44. The method of claim 42 or 43, wherein the first integrating further comprises a multiple cloning site.

45. The method of claim 44, wherein the fifth coding sequence encoding the sgRNA is located at the multiple cloning site.

46. The method of any one of claims 1-40, wherein the sgRNA is delivered into at least a portion of the population of cells by an expression vector.

47. The method of claim 46, wherein the expression vector comprises a U6 promoter operably linked to the fifth coding sequence encoding the sgRNA, a second 5′ paired site-specific recombination site and a second 3′ paired site-specific recombination site.

48. The method of claim 46 or 47, wherein the expression vector further comprises a multiple cloning site.

49. The method of claim 48, wherein the fifth coding sequence encoding the sgRNA is located at the multiple cloning site.

50. The method of any one of claims 46-49, wherein the expression vector further comprises a third promoter operably linked to a sixth coding sequence encoding a third detectable marker.

51. The method of claim 50, wherein the third detectable marker is an antibiotic resistance gene.

52. The method of claim 51, wherein the antibiotic resistant gene is a bls gene, hph gene, sh ble gene or geo gene.

53. The method of claim 50, wherein the third detectable marker is a fluorescent protein gene.

54. The method of claim 53, wherein the fluorescent protein is a GFP, RFP, tdtomato, mcherry, CFP, YFP, or BFP protein.

55. The method of claim 50, wherein the third detectable marker is a cell surface marker.

56. The method of claim 55, wherein the third detectable marker is luciferase or beta-galactosidase.

57. The method of any one of claims 1-56, wherein the first detectable marker, the second detectable marker and the third detectable marker are all different.

58. The method of any one of claims 1-57, wherein the expression vector further comprises an enhancer sequence.

59. The method of any one of claims 50-58, wherein the second 5′ paired site-specific recombination site and the second 3′ paired site-specific recombination site flank at least the sixth coding sequence encoding the third detectable marker.

60. The method of any one of claims 50-59, wherein the second 5′ site-specific recombination site and the second 3′ site-specific recombination site flank at least the sixth coding sequence encoding the third promoter and the third detectable marker.

61. The method of any one of claims 50-59, wherein the second 5′ paired site-specific recombination site and the second 3′ site-specific recombination site flank at least the third promoter, the sixth coding sequence encoding the third detectable marker and the enhancer sequence.

62. The method of any one of claims 50-59, wherein the second 5′ paired site-specific recombination site and the second 3′ paired site-specific recombination site flank at least the third promoter, sixth coding sequence encoding the third detectable marker, the enhancer sequence and the fifth coding sequence encoding the sgRNA.

63. The method of any one of claims 50-59, wherein the second 5′ paired site-specific recombination site and the second 3′ paired site-specific recombination site flank at least the third promoter, the sixth coding sequence encoding the third detectable marker, the enhancer sequence, the fifth coding sequence encoding the sgRNA and the U6 promoter.

64. The method of any one of claims 46-63, wherein the expression vector further comprises a seventh sequence encoding a fourth detectable marker.

65. The method of claim 64, wherein the fourth detectable marker is an antibiotic resistance gene.

66. The method of claim 65, wherein the antibiotic resistant gene is a bls gene, hph gene, sh ble gene or geo gene.

67. The method of claim 64, wherein the fourth detectable marker is a fluorescent protein gene.

68. The method of claim 67, wherein the fluorescence protein is a GFP, FRP, tdtomato, mcherry, CFP, YFP, or BFP protein.

69. The method of claim 64, wherein the fourth detectable marker is a cell surface marker.

70. The method of claim 64, wherein the fourth detectable marker is luciferase or beta-galactosidase.

71. The method of any one of claims 1-70, wherein the first detectable marker, the second detectable marker, the third detectable marker and the fourth detectable marker are all different.

72. The method of claim 71, wherein the seventh coding sequence encoding the fourth detectable marker is operably linked with the sixth coding sequence encoding the third detectable marker by a second spacer.

73. The method of claim 72, wherein the second spacer is an eighth coding sequence encoding a peptide.

74. The method of claim 73, wherein the peptide comprises a cleavage for a protease.

75. The method of claim 74, wherein the protease is an endogenous protease.

76. The method of any one of claims 73-75, wherein the peptide is a 2A peptide.

77. The method of claim 76, wherein the 2A peptide is a P2A peptide or a T2A peptide.

78. The method of claim 77, wherein the second spacer is an IRES.

79. The method of any one of claims 50-78, wherein the third promoter is a constitutive promoter, an inducible promoter or a tissue specific promoter.

80. The method of any one of claims 50-79, wherein the second 5′ paired site-specific recombination site and the second 3′ paired site-specific recombination site flank at least the sixth coding sequence encoding the third detectable marker.

81. The method of any one of claims 50-80, wherein the second 5′ paired site-specific recombination site and the second 3′ paired site-specific recombination site flank at least the sixth coding sequence encoding the third detectable marker, and the third promoter.

82. The method of any one of claims 50-80, wherein the second 5′ paired site-specific recombination site and the second 3′ paired site-specific recombination site flank at least the sixth coding sequence encoding the third detectable marker, the third promoter and the enhancer sequence.

83. The method of any one of claims 50-80, wherein the second 5′ paired site-specific recombination site and the second 3′ paired site-specific recombination site flank at least the sixth coding sequence encoding the third detectable marker, the third promoter, the enhancer sequence and the seventh coding sequence encoding the fourth detectable marker.

84. The method of any one of claims 50-80, wherein the second 5′ paired site-specific recombination site and the second 3′ paired site-specific recombination site flank at least the sixth coding sequence encoding the third detectable marker, the third promoter, the enhancer sequence, the seventh coding sequence encoding the fourth detectable marker and the fifth coding sequence encoding the sgRNA.

85. The method of any one of claims 50-80, wherein the second 5′ paired site-specific recombination site and the second 3′ paired recombination site flank at least the sixth sequence encoding the third detectable marker, the third promoter, the enhancer sequence, the seventh sequence encoding the fourth detectable marker, the fifth sequence encoding the sgRNA and the U6 promoter.

86. The method of any one of claims 50-83, wherein the expression vector is a lentiviral vector.

87. The method of any one of claim 1-86, wherein the genetic modification is a disruption of an endogenous gene, and wherein the sgRNA is designed to target a nucleic acid sequence of the endogenous gene.

88. The method of claim 87, further comprises:
repairing the double strand break by non-homologous end joining resulting in the disruption of the endogenous gene.

89. The method of any one of claims 1-86, wherein the genetic modification is an insertion of an exogenous nucleic acid into a target site targeted by the sgRNA.

90. The method of claim 89, further comprises:
introducing to the population of cells a donor sequence, wherein the donor sequence comprises the exogenous nucleic acid flanked by nucleic acid sequences that are homologous to the target site; and
repairing the double strand break by homologous recombination resulting in the insertion of the exogenous nucleic acid at the target site.

91. The method of claim 90, wherein the donor sequence can be introduced by calcium phosphate precipitation, liposome transfection, electroporation, or nanoparticles.

92. The method of claim 90 or 91, wherein the donor sequence is introduced to the population of cells prior to introducing the first integrating vector and the sgRNA.

93. The method of claim 90-92, wherein the donor sequence is introduced to the population of cells simultaneously when introducing the first integrating vector and the sgRNA.

94. The method of claim 90 or 91, wherein the donor sequence is introduced to the population of cells subsequent to the step of introducing the first integrating vector and the sgRNA.

95. The method of any one of claims 1-94, wherein the first recombinase is delivered into the population of the cells as a protein.

96. The method of any one of claims 1-94, wherein the first recombinase is delivered into the population of the cells by a ninth sequence encoding the first recombinase operably linked to a fourth promoter.

97. The method of claim 96, wherein the first recombinase is delivered into the population of the cells by a first AAV vector, wherein the first AAV vector comprises the ninth sequence encoding the first recombinase operably linked to the fourth promoter.

98. The method of claim 97, wherein the first recombinase is delivered into the population of the cells by a first integrase deficient lentiviral vector, wherein the first integrase deficient lentiviral vector comprises the ninth sequence encoding the first recombinase operably linked to the fourth promoter.

99. The method of any one of claims 1-98, the first recombinase is Cre.

100. The method of any one of claims 1-99, wherein the first site-specific recombination site and the second site specific recombination site comprise Lox sites.

101. The method of claim 100, wherein the Lox site is a LoxP, a Lox2272, or a Lox5171 site.

102. The method of any one of claim 101, wherein the first site-specific recombination site and the second site specific recombination site are identical.

103. The method of claim 46-102, wherein the second 5′ paired recombination site and the fourth site specific recombination site comprise Lox sites.

104. The method of claim 100, wherein the Lox site is a LoxP, a Lox2272, or a Lox5171 site.

105. The method of any one of claims 46-104, wherein the second 5′ paired recombination site and the fourth site specific recombination site are identical.

106. The method of any one of claims 1-105, wherein the first recombinase catalyzes excision of the nucleic acid between the second 5′ paired recombination site and the second 3′ paired recombination site.

107. The method of any one of claims 1-106, wherein the first site specific recombination site and the second site specific recombination site are different from the second 5′ paired recombination site and the second 3′ paired recombination site.

108. The method of claim 46-102, wherein a second recombinase catalyzes excision of the nucleic acid between the second 5′ paired recombination site and the second 3′ paired recombination site.

109. The method of claim 108, wherein the second recombinase is delivered into the population of the cells as a protein.

110. The method of claim 108, wherein the second recombinase is delivered into the population of the cells by a tenth sequence encoding the second recombinase operably linked to a fifth promoter.

111. The method of claim 110, wherein the second recombinase is delivered into the population of the cells by a second AAV vector, wherein the second AAV vector comprises the tenth sequence encoding the second recombinase operably linked to the fifth promoter.

112. The method of claim 110, wherein the second recombinase is delivered into the population of the cells by a second integrase deficient lentiviral vector, wherein the second integrase deficient lentiviral vector comprises the tenth sequence encoding the second recombinase operably linked to the fifth promoter.

113. The method of any one of claims 1-112, wherein the first recombinase is Cre, FLP, ΦC31 or Dre.

114. The method of any one of claims 1-113, wherein the second recombinase is Cre, FLP, ΦC31 or Dre.

115. The method of any one of claims 1-114, wherein the first recombinase and the second recombinase are different.

116. A first integrating vector, comprising:
a promoter operably linked to a nucleotide sequence encoding a Cas protein;
at least two copies of a site-specific recombination site; and
at least one nucleotide sequence encoding a selectable marker.

117. The first integrating vector of claim 116, wherein the nucleotide sequence encoding a Cas protein is fused with the nucleotide sequence encoding the selectable marker.

118. The first integrating vector of claim 116 or 117, further comprising a spacer sequence located between the nucleotide sequence encoding a Cas protein and the nucleotide sequence encoding the selectable marker.

119. The first integrating vector of any one of claims 116-118, further comprising an enhancer sequence.

120. The first integrating vector of any one of claims 116-119, wherein the recombinogenic vector is a lentiviral vector.

121. The first integrating vector of any one of claims 116-120, wherein the promoter is a constitutive promoter.

122. The first integrating vector of any one of claims 116-120, wherein the promoter is an inducible promoter.

123. The first integrating vector of any one of claims 116-120, wherein the promoter is a tissue specific promoter.

124. The first integrating vector of claim 118, wherein the spacer is a nucleotide sequence encoding a peptide.

125. The first integrating vector of claim 124, wherein the peptide is a 2A peptide.

126. The first integrating vector of claim 124, therein the peptide comprises a cleavage site for a protease.

127. The first integrating vector of claim 126, wherein the protease is an endogenous protease.

128. The first integrating vector of claim 118, wherein the spacer is an IRES.

129. The first integrating vector of any one of claims 116-128, wherein the selectable marker is a nucleotide sequence encoding an antibiotic resistant gene.

130. The first integrating vector of claim 129, wherein the antibiotic resistant gene is bls gene, hph gene, sh ble gene or neo gene.

131. The first integrating vector of any one of claims 116-128, wherein the selectable marker is a nucleotide sequence encoding a fluorescence protein.

132. The first integrating vector of claim 131, wherein the fluorescence protein is GFP, FRP, tdtomato, mcherry, CFP, YFP, or BFP.

133. The first integrating recombinogenic vector of any one of claims 116-128, wherein the selectable marker is a nucleotide sequence encoding a cell surface marker.

134. The first integrating vector of any one of claims 116-128, wherein the selectable marker is luciferase or beta-galactosidase.

135. The first integrating vector of any one of claims 116-134, wherein at least the nucleotide sequence encoding a Cas protein is located between the two copies of the site specific recombination site.

136. The first integrating vector of any one of claims 116-135, wherein at least the nucleotide sequence encoding a Cas protein and the nucleotide sequence encoding the selectable marker is located between the two copies of the specific recombination site.

137. The first integrating vector of any one of claims 116-136, wherein the two copies of the site specific recombination site can be recognized by Cre, FLP, ΦC31 or Dre.

138. A second integrating vector, comprising:
at least two copies of a site-specific recombination site;
a first promoter operably linked to at least one nucleotide sequence encoding an sgRNA; and
a second promoter operably linked to at least one nucleotide sequence encoding a selectable marker.

139. The second integrating vector of claim 138, further comprising an enhancer sequence.

140. The second integrating vector of claim 138 or 139, wherein the recombinogenic vector is a lentiviral vector.

141. The second integrating vector of any one of claims 138-140, wherein the first promoter is a U6 promoter.

142. The second integrating vector of any one of claims 138-141, wherein the second promoter is a constitutive promoter.

144. The second integrating vector of any one of claims 138-141, wherein the second promoter is an inducible promoter.

145. The second integrating vector of any one of claims 138-141, wherein the second promoter is tissue specific promoter.

146. The second integrating vector of any one of claims 138-145, further comprising a multiple cloning site, and wherein the sgRNA is located at the multiple cloning site.

147. The second integrating vector of any one of claims 138-146, wherein the selectable marker is a nucleotide sequence encoding an antibiotic resistant gene;

148. The second integrating vector of claim 147, wherein the antibiotic resistant gene is a bls gene, hph gene, sh ble gene or neo gene.

149. The second integrating vector of any of claims 138-148, wherein the selectable marker is a fluorescence protein.

150. The second integrating vector of claim 149, wherein the fluorescence protein is a GFP, FRP, tdtomato, mcherry, CFP, YFP, or BFP protein.

151. The second integrating vector of any one of claims 138-146, wherein the selectable marker is a cell surface marker.

152. The second integrating vector of any one of claims 138-146, wherein the selectable marker is a luciferase or beta-galactosidase.

153. The second integrating vector of any one of claims 138-152, further comprising a nucleotide sequence encoding a gene flanked by two homologous nucleotide sequences to a target site.

154. The second integrating vector of claim any one of claims 138-153, wherein at least the nucleotide encoding the selectable marker is located between the two copies of the site specific recombination site.

155. The second integrating vector of any one of claims 138-154, wherein the two copies of the site specific recombination site can be recognized by Cre, FLP, ΦC31 or Dre.

156. The second integrating vector of any one of claims 138-154, wherein the sgRNA further comprises a bar code sequence.

157. A kit for producing genetically modified cells, comprising:
(i) a first integrating vector, comprising:
at least two copies of a first site-specific recombination site;
a promoter operably linked to a nucleotide sequence encoding a Cas protein; and
at least one nucleotide sequence encoding a selectable marker;
(ii) a second integrating vector, comprising
at least two copies of a second site-specific recombination site;
a first promoter operably linked to a nucleotide sequence encoding an sgRNA; and
a second promoter operably linked to at least one nucleotide sequence encoding a selectable marker;
(iii) a third vector, comprising a promoter operably linked to a nucleotide sequence encoding a first recombinase, wherein the first recombinase recognizes the first site specific recombination site of (i); and
(ii) a fourth vector, comprising a promoter operably linked to a nucleotide sequence encoding a second recombinase, wherein the second recombinase recognizes the second site specific recombination site of (ii).

158. The kit of claim 157, where in the first site specific recombination site of (i) is different from the second site specific recombination site of (ii).

159. The kit of claim 157 or 158, wherein the third vector is an AAV vector.

160. The kit of any one of claims 157-159, wherein the third vector is an integrase deficient lentiviral vector.

161. The kit of any one of claims 157-160, wherein the fourth vector is an AAV vector.

162. The kit of any one of claims 157-161, wherein the fourth vector is an integrase deficient lentiviral vector.

163. The kit of any one of claims 157-162, wherein the second integrating vector further comprises a multiple cloning site.

164. The kit of claim 163, wherein the nucleotide sequence encoding the sgRNA is located at the multiple cloning cite.

165. The kit of any one of claims 157-164, wherein the nucleotide sequence encoding the sgRNA is designed to recognize a target sequence.

166. The kit of any one of claims 157-165, further comprising a donor nucleotide sequence.

167. The kit of claim 164, wherein the donor nucleotide sequence comprises a nucleotide sequence to be inserted at the target sequence flanked by two homologous sequences to the target sequence.

168. A method of screening a population of genetically modified cells for a candidate target gene, comprising:
(i) providing a population of tumor cells;
(ii) introducing a first integration vector into at least a portion of the population of tumor cells,
wherein the first integration vector comprises a first nucleic acid sequence comprising a first promoter operably linked to a Cas protein coding sequence encoding a Cas protein; and at least a first 3′ site-specific recombination site located 3′ to the Cas coding sequence, and
wherein the first integrating vector is capable of integration into the genomes of at least a portion of the population of cells;
(iii) introducing a plurality of second integration vectors into at least a portion of the population of tumor cells,
wherein each of the plurality of second integration vectors comprises a second nucleic acid sequence encoding an sgRNA,
wherein the sgRNA comprises a nucleotide sequence comprising a bar code that corresponds to a candidate target gene, and
wherein the sgRNA is capable of guiding the Cas protein to a target site in the genomes of at least a portion of the population of cells, and wherein the Cas protein is capable of double-stranded DNA cleavage at the target site;
(iv) culturing the population of tumor cells for a time sufficient for (a) integration of the first integrating vector into the genomes of at least a portion of the population of cells;
and (b) induction of a genetic modification at the target site in the genomes of at least a portion of the population of cells by double-stranded DNA cleavage by the Cas protein and the sgRNA; and
(v) introducing a first recombinase into at least a portion of the population of cells, wherein the first recombinase catalyzes recombination between the first 3′ site-specific recombination site and a first 5′ site-specific recombination site located 5′ to at least the Cas protein coding sequence, thereby causing excision of the Cas protein coding sequence from the genomes of at least a portion of the population of cells.

169. The method of claim 168, further comprising:
(vi) grafting a portion of the modified tumor cells of the population onto a mammal;
(vii) treating the mammal with a monoclonal antibody sufficient to generate an adaptive immune response in the mammal; and
(viii) isolating the grafted modified tumor cells and sequencing the genomic DNA of the modified tumor cells.

170. The method of claim 168 or 169, wherein each of the first integration vector and each of the plurality of second integration vectors comprises a a replication defective retroviral vector derived from a primate lentivirus.

171. The method of any one of claims 168-170, wherein the monoclonal antibody is selected from an anti-CTLA4 and an anti-PD-1 monoclonal antibody.

172. The method of any one of claims 168-171, wherein the mammal is murine.

173. The method of any one of claims 168-172, wherein the sgRNA comprises at least 10, at least 25, at least 50, at least 75, at least 100, at least 250, at least 500, at least 750, at least 1,000, or at least 5,000 sgRNAs, wherein each sgRNA comprises a bar code that corresponds to a candidate target gene, and wherein no two bar codes are identical.

174. A kit for producing a population of genetically modified tumor cells, comprising:
(i) a first integrating vector, comprising:
at least two copies of a first site-specific recombination site;
a promoter operably linked to a nucleotide sequence encoding a Cas protein; and
at least one nucleotide sequence encoding a selectable marker;
(ii) a plurality of second integrating vectors, each comprising at least two copies of a second site-specific recombination site;
a first promoter operably linked to a nucleotide sequence encoding an sgRNA comprising a nucleotide sequence comprising a bar code that corresponds to a candidate target gene; and
a second promoter operably linked to at least one nucleotide sequence encoding a selectable marker; a plurality of second integration vectors into at least a portion of the population of tumor cells,
(iii) a third vector, comprising a promoter operably linked to a nucleotide sequence encoding a first recombinase, wherein the first recombinase recognizes the first site specific recombination site of (i); and
(ii) a fourth vector, comprising a promoter operably linked to a nucleotide sequence encoding a second recombinase, wherein the second recombinase recognizes the second site specific recombination site of (ii).

175. The kit of claim 174, wherein each of the first integration vector and each of the plurality of second integration vectors comprises a a replication defective retroviral vector derived from a primate lentivirus.

176. The kit of claim 174 or 175, wherein the third vector is an AAV vector.

177. The kit of any one of claims 174-176, wherein the third vector is an integrase deficient lentiviral vector.

178. The kit of any one of claims 174-177, wherein the fourth vector is an AAV vector.

179. The kit of any one of claims 174-178, wherein the fourth vector is an integrase deficient lentiviral vector.


SEQUENCE LISTING (10 sequences)

SEQ ID NO: 1 - DNA, 9 bp, Artificial Sequence
  Synthetic polynucleotide
gagcatatc

SEQ ID NO: 2 - RNA, 9 nt, Artificial Sequence
  Synthetic polynucleotide
gauaugcuc

SEQ ID NO: 3 - DNA, 20 bp, Artificial Sequence
  Synthetic polynucleotide
gcgaggtattcggctccgcg

SEQ ID NO: 4 - DNA, 20 bp, Artificial Sequence
  Synthetic polynucleotide
ccacattacggacgatgcaa

SEQ ID NO: 5 - DNA, 20 bp, Artificial Sequence
  Synthetic polynucleotide
agtatactcacgccacccac

SEQ ID NO: 6 - DNA, 13229 bp, Artificial Sequence
  Synthetic polynucleotide
acaagtttgtacaaaaaagttggcacccccaactttatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaacttgtacaaagtggttgatatcggtaagcctatccctaaccctctcctcggtctcgattctacgtagtaatgaactagtaccggttaagtcgacaatcaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccatgcattgcatctcaattagtcagcaaccaggtgtggaaagtccccaggctccccagcaggcagaagtatgcaaagcatgcgtctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctttctagaggtaccaccatggtgagcaagggcgaggagctgttcaccggggtggtgcccatcctggtcgagctggacggcgacgtaaacggccacaagttcagcgtgtctggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtcctaaggcgcgccgttaacgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgaggcctgcaggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggctagcatcgattgatca

SEQ ID NO: 7 - DNA, 13244 bp, Artificial Sequence
  Synthetic polynucleotide
ctcgaggcctgcaggtgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggctagcatcgattgatcaacaagtttgtacaaaaaagttggcacccccaactttatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgatggtgtccaaaggggaggaactcttcactggcgttgtcccaattctggtggagctggacggcgacgtaaatggccacaagtttagcgtgagtggggagggagagggtgacgcgacatacggcaagctgacactgaaatttatttgtacgaccgggaaactgcccgtgccctggcccacacttgtgacgactttgacctatggcgtccagtgcttttccaggtatccagaccatatgaagcagcacgacttctttaaaagcgctatgccggaagggtacgttcaggagcgcacgattttttttaaggacgatggtaattataagacccgagccgaggttaaatttgagggagataccctggtgaatcgcatcgaactgaagggcattgatttcaaggaggatggcaatattctcggccacaaacttgagtacaactacaattctcacaacgtatacatcatggcggataaacagaagaacggaatcaaggtgaacttcaagattaggcacaacattgaagatggcagcgttcagctggccgaccactatcaacagaatacccctattggggatggccctgtgctcttgcccgataaccactatctgagcacccagagcgcgctgagcaaagatccaaatgaaaagcgggaccatatggtgctgttggagtttgtcactgccgcaggaatcacactgggcatggacgagctgtacaagtcttaacttgtacaaagtggttgatatcggtaagcctatccctaaccctctcctcggtctcgattctacgtagtaatgaactagtaccggttaagtcgacaatcaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatgggatcaattcaccatgggaataacttcgtatagcatacattatacgaagttatgctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcatacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccatgcattgcatctcaattagtcagcaaccaggtgtggaaagtccccaggctccccagcaggcagaagtatgcaaagcatgcgtctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctttctagaggtaccaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtagtaaggcgcgccgttaacgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccgg

SEQ ID NO: 8 - DNA, 9180 bp, Artificial Sequence
  Synthetic polynucleotide
gatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgcttttttcgtctcagtttgagagctagaaatagcaagttcaaataaggctagtccgttatcaacttgaaaaagtggcaccgagtcggtgcttttttgaattcaagcttggcgtaactagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagggggcccgggataacttcgtatagtacacattatacgaagttattgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtttcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccgctagcctgcagggatccggcgcaacaaacttctctctgctgaaacaagccggagatgtcgaagagaatcctggaccggctagcatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagaccctgtaccccgctgacggcggcctggaaggcagagccgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacagataaataacttcgtatagtacacattatacgaagttatacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcacc

SEQ ID NO: 9 - DNA, 9180 bp, Artificial Sequence; identical to SEQ ID NO: 8
  Synthetic polynucleotide

SEQ ID NO: 10 - DNA, 7398 bp, Artificial Sequence
  Synthetic polynucleotide
accggttaagtcgacaatcaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccatgcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggctaatctcctgaccgtgcatcagaatctgcctgccctgcccgtcgacgcaacaagcgatgaagtccgcaagaatctcatggacatgttcagggacagacaggccttttccgagcacacctggaagatgctgctgagcgtgtgcaggtcctgggctgcttggtgtaagctgaacaacagaaagtggttcccagctgagccagaggacgtgcgggattacctgctgtacctgcaggcccgcggactggctgtgaagacaatccagcagcacctgggccagctgaacatgctgcacaggagaagcggactgccccggcctagcgactccaacgccgtgagcctggtcatgcggcgcatcaggaaggagaacgtggatgccggcgagagagctaagcaggccctggctttcgagaggaccgactttgatcaggtgagatctctgatggagaacagcgacaggtgccaggatatcagaaacctggcctttctgggaatcgcttacaacaccctgctgagaatcgccgagatcgctcggatccgcgtgaaggacatctctcggacagatggcggacgcatgctgatccacatcggcaggaccaagacactggtgtccaccgccggcgtggagaaggctctgtctctgggagtgacaaagctggtggagagatggatctctgtgagcggcgtggccgacgatcctaacaactacctgttctgtagggtgagaaagaacggagtggccgctccatccgctacctctcagctgagcacacgggccctggagggcatctttgaggctacccaccgcctgatctacggcgccaaggacgattctggacagcggtacctggcttggtccggacactctgctcgcgtgggagctgctcgggatatggcccgcgctggcgtgagcatcccagagatcatgcaggccggcggatggacaaacgtgaacatcgtgatgaactacattagaaatctggatagcgaaactggggcaatggtgcggctgctggaggatggggactgatagtaatgaactagt